SEQUENCE LISTING (93 sequences)

SEQ ID NO: 1 - protein, 4374 aa, Homo sapiens
MKVDRTKLKKTPTEAPADCRALIDKLKVCNDEQLLLELQQIKTWNIGKCELYHWVDLLDRFDGILADAGQTVENMSWMLVCDRPEREQLKMLLLAVLNFTALLIEYSFSRHLYSSIEHLTTLLASSDMQVVLAVLNLLYVFSKRSNYITRLGSDKRTPLLTRLQHLAESWGGKENGFGLAECCRDLHMMKYPPSATTLHFEFYADPGAEVKIEKRTTSNTLHYIHIEQLDKISESPSEIMESLTKMYSIPKDKQMLLFTHIRLAHGFSNHRKRLQAVQARLHAISILVYSNALQESANSILYNGLIEELVDVLQITDKQLMEIKAASLRTLTSIVHLERTPKLSSIIDCTGTASYHGFLPVLVRNCIQAMIDPSMDPYPHQFATALFSFLYHLASYDAGGEALVSCGMMEALLKVIKFLGDEQDQITFVTRAVRVVDLITNLDMAAFQSHSGLSIFIYRLEHEVDLCRKECPFVIKPKIQRPNTTQEGEEMETDMDGVQCIPQRAALLKSMLNFLKKAIQDPAFSDGIRHVMDGSLPTSLKHIISNAEYYGPSLFLLATEVVTVFVFQEPSLLSSLQDNGLTDVMLHALLIKDVPATREVLGSLPNVFSALCLNARGLQSFVQCQPFERLFKVLLSPDYLPAMRRRRSSDPLGDTASNLGSAVDELMRHQPTLKTDATTAIIKLLEEICNLGRDPKYICQKPSIQKADGTATAPPPRSNHAAEEASSEDEEEEEVQAMQSFNSTQQNETEPNQQVVGTEERIPIPLMDYILNVMKFVESILSNNTTDDHCQEFVNQKGLLPLVTILGLPNLPIDFPTSAACQAVAGVCKSILTLSHEPKVLQEGLLQLDSILSSLEPLHRPIESPGGSVLLRELACAGNVADATLSAQATPLLHALTAAHAYIMMFVHTCRVGQSEIRSISVNQWGSQLGLSVLSKLSQLYCSLVWESTVLLSLCTPNSLPSGCEFGQADMQKLVPKDEKAGTTQGGKRSDGEQDGAAGSMDASTQGLLEGIGLDGDTLAPMETDEPTASDSKGKSKITPAMAARIKQIKPLLSASSRLGRALAELFGLLVKLCVGSPVRQRRSHHAASTTTAPTPAARSTASALTKLLTKGLSWQPPPYTPTPRFRLTFFICSVGFTSPMLFDERKYPYHLMLQKFLCSGGHNALFETFNWALSMGGKVPVSEGLEHSDLPDGTGEFLDAWLMLVEKMVNPTTVLESPHSLPAKLPGGVQNFPQFSALRFLVVTQKAAFTCIKNLWNRKPLKVYGGRMAESMLAILCHILRGEPVIRERLSKEKEGSRGEEDTGQEEGGSRREPQVNQQQLQQLMDMGFTREHAMEALLNTSTMEQATEYLLTHPPPIMGGVVRDLSMSEEDQMMRAIAMSLGQDIPMDQRAESPEEVACRKEEEERKAREKQEEEEAKCLEKFQDADPLEQDELHTFTDTMLPGCFHLLDELPDTVYRVCDLIMTAIKRNGADYRDMILKQVVNQVWEAADVLIKAALPLTTSDTKTVSEWISQMATLPQASNLATRILLLTLLFEELKLPCAWVVESSGILNVLIKLLEVVQPCLQAAKEQKEVQTPKWITPVLLLIDFYEKTAISSKRRAQMTKYLQSNSNNWRWFDDRSGRWCSYSASNNSTIDSAWKSGETSVRFTAGRRRYTVQFTTMVQVNEETGNRRPVMLTLLRVPRLNKNSKNSNGQELEKTLEESKEMDIKRKENKGNDTPLALESTNTEKETSLEETKIGEILIQGLTEDMVTVLIRACVSMLGVPVDPDTLHATLRLCLRLTRDHKYAMMFAELKSTRMILNLTQSSGFNGFTPLVTLLLRHIIEDPCTLRHTMEKVVRSAATSGAGSTTSGVVSGSLGSREINYILRVLGPAACRNPDIFTEVANCCIRIALPAPRGSGTASDDEFENLRIKGPNAVQLVKTTPLKPSPLPVIPDTIKEVIYDMLNALAAYHAPEEADKSDPKPGVMTQEVGQLLQDMGDDVYQQYRSLTRQSSDFDTQSGFSINSQVFAADGASTETSASGTSQGEASTPEESRDGKKDKEGDRASEEGKQKGKGSKPLMPTSTILRLLAELVRSYVGIATLIANYSYTVGQSELIKEDCSVLAFVLDHLLPHTQNAEDKDTPALARLFLASLAAAGSGTDAQVALVNEVKAALGRALAMAESTEKHARLQAVMCIISTIMESCPSTSSFYSSATAKTQHNGMNNIIRLFLKKGLVNDLARVPHSLDLSSPNMANTVNAALKPLETLSRIVNQPSSLFGSKSASSKNKSEQDAQGASQDSSSNQQDPGEPGEAEVQEEDHDVTQTEVADGDIMDGEAETDSVVIAGQPEVLSSQEMQVENELEDLIDELLERDGGSGNSTIIVSRSGEDESQEDVLMDEAPSNLSQASTLQANREDSMNILDPEDEEEHTQEEDSSGSNEDEDDSQDEEEEEEEDEEDDQEDDEGEEGDEDDDDDGSEMELDEDYPDMNASPLVRFERFDREDDLIIEFDNMFSSATDIPPSPGNIPTTHPLMVRHADHSSLTLGSGSSTTRLTQGIGRSQRTLRQLTANTGHTIHVHYPGNRQPNPPLILQRLLGPSAAADILQLSSSLPLQSRGRARLLVGNDDVHIIARSDDELLDDFFHDQSTATSQAGTLSSIPTALTRWTEECKVLDAESMHDCVSVVKVSIVNHLEFLRDEELEERREKRRKQLAEEETKITDKGKEDKENRDQSAQCTASKSNDSTEQNLSDGTPMPDSYPTTPSSTDAATSESKETLGTLQSSQQQPTLPTPPALGEVPQELQSPAGEGGSSTQLLMPVEPEELGPTRPSGEAETTQMELSPAPTITSLSPERAEDSDALTAVSSQLEGSPMDTSSLASCTLEEAVGDTSAAGSSEQPRAGSSTPGDAPPAVAEVQGRSDGSGESAQPPEDSSPPASSESSSTRDSAVAISGADSRGILEEPLPSTSSEEEDPLAGISLPEGVDPSFLAALPDDIRREVLQNQLGIRPPTRTAPSTNSSAPAVVGNPGVTEVSPEFLAALPPAIQEEVLAQQRAEQQRRELAQNASSDTPMDPVTFIQTLPSDLRRSVLEDMEDSVLAVMPPDIAAEAQALRREQEARQRQLMHERLFGHSSTSALSAILRSPAFTSRLSGNRGVQYTRLAVQRGGTFQMGGSSSHNRPSGSNVDTLLRLRGRLLLDHEALSCLLVLLFVDEPKLNTSRLHRVLRNLCYHAQTRHWVIRSLLSILQRSSESELCIETPKLTTSEEKGKKSSKSCGSSSHENRPLDLLHKMESKSSNQLSWLSVSMDAALGCRTNIFQIQRSGGRKHTEKHASGGSTVHIHPQAAPVVCRHVLDTLIQLAKVFPSHFTQQRTKETNCESDRERGNKACSPCSSQSSSSGICTDFWDLLVKLDNMNVSRKGKNSVKSVPVSAGGEGETSPYSLEASPLGQLMNMLSHPVIRRSSLLTEKLLRLLSLISIALPENKVSEAQANSGSGASSTTTATSTTSTTTTTAASTTPTPPTAPTPVTSAPALVAATAISTIVVAASTTVTTPTTATTTVSISPTTKGSKSPAKVSDGGSSSTDFKMVSSGLTENQLQLSVEVLTSHSCSEEGLEDAANVLLQLSRGDSGTRDTVLKLLLNGARHLGYTLCKQIGTLLAELREYNLEQQRRAQCETLSPDGLPEEQPQTTKLKGKMQSRFDMAENVVIVASQKRPLGGRELQLPSMSMLTSKTSTQKFFLRVLQVIIQLRDDTRRANKKAKQTGRLGSSGLGSASSIQAAVRQLEAEADAIIQMVREGQRARRQQQAATSESSQSEASVRREESPMDVDQPSPSAQDTQSIASDGTPQGEKEKEERPPELPLLSEQLSLDELWDMLGECLKELEESHDQHAVLVLQPAVEAFFLVHATERESKPPVRDTRESQLAHIKDEPPPLSPAPLTPATPSSLDPFFSREPSSMHISSSLPPDTQKFLRFAETHRTVLNQILRQSTTHLADGPFAVLVDYIRVLDFDVKRKYFRQELERLDEGLRKEDMAVHVRRDHVFEDSYRELHRKSPEEMKNRLYIVFEGEEGQDAGGLLREWYMIISREMFNPMYALFRTSPGDRVTYTINPSSHCNPNHLSYFKFVGRIVAKAVYDNRLLECYFTRSFYKHILGKSVRYTDMESEDYHFYQGLVYLLENDVSTLGYDLTFSTEVQEFGVCEVRDLKPNGANILVTEENKKEYVHLVCQMRMTGAIRKQLAAFLEGFYEIIPKRLISIFTEQELELLISGLPTIDIDDLKSNTEYHKYQSNSIQIQWFWRALRSFDQADRAKFLQFVTGTSKVPLQGFAALEGMNGIQKFQIHRDDRSTDRLPSAHTCFNQLDLPAYESFEKLRHMLLLAIQECSEGFGLA

SEQ ID NO: 2 - protein, 381 aa, Homo sapiens
MAAFGRQVLDWHRLIPLTWACMARQTPHLGEQRRTTASLLRKLTTASNGGVIEELSCVRSNNYVQEPECRRNLVQCLLEKQGTPVVQGSLELERVMSSLLDMGFSNAHINELLSVRRGASLQQLLDIISEFILLGLNPEPVCVVLKKSPQLLKLPIMQMRKRSSYLQKLGLGEGKLKRVLYCCPEIFTMRQQDINDTVRLLKEKCLFTVQQVTKILHSCPSVLREDLGQLEYKFQYAYFRMGIKHPDIVKSEYLQYSLTKIKQRHIYLERLGRYQTPDKKGQTQIPNPLLKDILRVSEAEFLARTACTSVEEFQVFKKLLAREEEESESSTSDDKRASLDEDEDDDDEEDNDEDDNDEDDDDEDDDEAEDNDEDEDDDEEE

SEQ ID NO: 3 - protein, 1121 aa, Homo sapiens
MSLENEDKRARTRSKALRGPPETTAADLSCPTPGCTGSGHVRGKYSRHRSLQSCPLAKKRKLEGAEAEHLVSKRKSHPLKLALDEGYGVDSDGSEDTEVKDASVSDESEGTLEGAEAETSGQDEIHRPETAEGRSPVKSHFGSNPIGSATASSKGSYSSYQGIIATSLLNLGQIAEETLVEEDLGQAAKPGPGIVHLLQEAAEGAASEEGEKGLFIQPEDAEEVVEVTTERSQDLCPQSLEDAASEESSKQKGILSHEEEDEEEEEEEEEEEEDEEEEEEEEEEEEEEEEEEEEEEEEEEEEEEEEAAPDVIFQEDTSHTSAQKAPELRGPESPSPKPEYSVIVEVRSDDDKDEDTHSRKSTVTDESEMQDMMTRGNLGLLEQAIALKAEQVRTVCEPGCPPAEQSQLGLGEPGKAAKPLDTVRKSYYSKDPSRAEKREIKCPTPGCDGTGHVTGLYPHHRSLSGCPHKDRIPPEILAMHENVLKCPTPGCTGQGHVNSNRNTHRSLSGCPIAAAEKLAKSHEKQQPQTGDPSKSSSNSDRILRPMCFVKQLEVPPYGSYRPNVAPATPRANLAKELEKFSKVTFDYASFDAQVFGKRMLAPKIQTSETSPKAFQCFDYSQDAEAAHMAATAILNLSTRCWEMPENLSTKPQDLPSKSVDIEVDENGTLDLSMHKHRKRENAFPSSSSCSSSPGVKSPDASQRHSSTSAPSSSMTSPQSSQASRQDEWDRPLDYTKPSRLREEEPEESEPAAHSFASSEADDQEVSEENFEERKYPGEVTLTNFKLKFLSKDIKKELLTCPTPGCDGSGHITGNYASHRSLSGCPLADKSLRNLMAAHSADLKCPTPGCDGSGHITGNYASHRSLSGCPRAKKSGVKVAPTKDDKEDPELMKCPVPGCVGLGHISGKYASHRSASGCPLAARRQKEGSLNGSSFSWKSLKNEGPTCPTPGCDGSGHANGSFLTHRSLSGCPRATFAGKKGKLSGDEVLSPKFKTSDVLENDEEIKQLNQEIRDLNESNSEMEAAMVQLQSQISSMEKNLKNIEEENKLIEEQNEALFLELSGLSQALIQSLANIRLPHMEPICEQNFDAYVSTLTDMYSNQDPENKDLLESIKQAVRGIQV

SEQ ID NO: 4 - protein, 1099 aa, Homo sapiens
MGKLIRMGPQERWLLRTKRLHWSRLLFLLGMLIIGSTYQHLRRPRGLSSLWAAVSSHQPIKLASRDLSSEEMMMMSSSPSKPSSEMGGKMLVPQASVGSDEATLSMTVENIPSMPKRTAKMIPTTTKNNYSPTAAGTERRKEDTPTSSRTLTYYTSTSSRQIVKKYTPTPRGEMKSYSPTQVREKVKYTPSPRGRRVGTYVPSTFMTMETSHAITPRTTVKDSDITATYKILETNSLKRIMEETTPTTLKGMFDSTPTFLTHEVEANVLTSPRSVMEKNNLFPPRRVESNSSAHPWGLVGKSNPKTPQGTVLLHTPATSEGQVTISTMTGSSPAETKAFTAAWSLRNPSPRTSVSAIKTAPAIVWRLAKKPSTAPSTSTTPTVRAKLTMQVHHCVVVKPTPAMLTTPSPSLTTALLPEELSPSPSVLPPSLPDLHPKGEYPPDLFSVEERRQGWVVLHVFGMMYVFVALAIVCDEYFVPALGVITDKLQISEDVAGATFMAAGGSAPELFTSLIGVFISHSNVGIGTIVGSAVFNILFVIGTCSLFSREILNLTWWPLFRDVSFYILDLIMLILFFLDSLIAWWESLLLLLAYAFYVFTMKWNKHIEVWVKEQLSRRPVAKVMALEDLSKPGDGAIAVDELQDNKKLKLPSLLTRGSSSTSLHNSTIRSTIYQLMLHSLDPLREVRLAKEKEEESLNQGARAQPQAKAESKPEEEEPAKLPAVTVTPAPVPDIKGDQKENPGGQEDVAEAESTGEMPGEEGETAGEGETEEKSGGETQPEGEGETETQGKGEECEDENEAEGKGDNEGEDEGEIHAEDGEMKGNEGETESQELSAENHGEAKNDEKGVEDGGGSDGGDSEEEEEEEEEQEEEEEEEEQEEEEEEEEEEEEKGNEEPLSLDWPETRQKQAIYLFLLPIVFPLWLTVPDVRRQESRKFFVFTFLGSIMWIAMFSYLMVWWAHQVGETIGISEEIMGLTILAAGTSIPDLITSVIVARKGLGDMAVSSSVGSNIFDITVGLPVPWLLFSLINGLQPVPVSSNGLFCAIVLLFLMLLFVISSIASCKWRMNKILGFTMFLLYFVFLIISVMLEDRIISCPVSV

SEQ ID NO: 5 - protein, 1186 aa, Homo sapiens
MEVDTEEKRHRTRSKGVRVPVEPAIQELFSCPTPGCDGSGHVSGKYARHRSVYGCPLAKKRKTQDKQPQEPAPKRKPFAVKADSSSVDECDDSDGTEDMDEKEEDEGEEYSEDNDEPGDEDEEDEEGDREEEEEIEEEDEDDDEDGEDVEDEEEEEEEEEEEEEEEENEDHQMNCHNTRIMQDTEKDDNNNDEYDNYDELVAKSLLNLGKIAEDAAYRARTESEMNSNTSNSLEDDSDKNENLGRKSELSLDLDSDVVRETVDSLKLLAQGHGVVLSENMNDRNYADSMSQQDSRNMNYVMLGKPMNNGLMEKMVEESDEEVCLSSLECLRNQCFDLARKLSETNPQERNPQQNMNIRQHVRPEEDFPGRTPDRNYSDMLNLMRLEEQLSPRSRVFASCAKEDGCHERDDDTTSVNSDRSEEVFDMTKGNLTLLEKAIALETERAKAMREKMAMEAGRRDNMRSYEDQSPRQLPGEDRKPKSSDSHVKKPYYGKDPSRTEKKESKCPTPGCDGTGHVTGLYPHHRSLSGCPHKDRVPPEILAMHESVLKCPTPGCTGRGHVNSNRNSHRSLSGCPIAAAEKLAKAQEKHQSCDVSKSSQASDRVLRPMCFVKQLEIPQYGYRNNVPTTTPRSNLAKELEKYSKTSFEYNSYDNHTYGKRAIAPKVQTRDISPKGYDDAKRYCKDPSPSSSSTSSYAPSSSSNLSCGGGSSASSTCSKSSFDYTHDMEAAHMAATAILNLSTRCREMPQNLSTKPQDLCATRNPDMEVDENGTLDLSMNKQRPRDSCCPILTPLEPMSPQQQAVMNNRCFQLGEGDCWDLPVDYTKMKPRRIDEDESKDITPEDLDPFQEALEERRYPGEVTIPSPKPKYPQCKESKKDLITLSGCPLADKSIRSMLATSSQELKCPTPGCDGSGHITGNYASHRSLSGCPRAKKSGIRIAQSKEDKEDQEPIRCPVPGCDGQGHITGKYASHRSASGCPLAAKRQKDGYLNGSQFSWKSVKTEGMSCPTPGCDGSGHVSGSFLTHRSLSGCPRATSAMKKAKLSGEQMLTIKQRASNGIENDEEIKQLDEEIKELNESNSQMEADMIKLRTQITTMESNLKTIEEENKVIEQQNESLLHELANLSQSLIHSLANIQLPHMDPINEQNFDAYVTTLTEMYTNQDRYQSPENKALLENIKQAVRGIQV

SEQ ID NO: 6 - protein, 663 aa, Homo sapiens
MAHLRSPSGFGDPGKKDQKESEEELEEEEEEEEVEEEEEEVEEEEEEVEEEEEEVVEEELVGEEQELEAPETFSEEYLWKVTDIGDYDDDFPDVRPRLASIVSPSLTSTFVPSQSATSTETPSASPPSSTSSHKSFPKIFQTFRKDMSEMSIDRNIHRNLSPGIPVSVQTEESWLQDLSDKVQSRKKASKEKAEPECLASKLREKWVINPEESKLNILYELEFKEDFITLFEPSLRTLPSIGPPSILAYKEESSNLGINFKDEEEETSPKCEFCGSDLRAFFSNVDVSSEPKGHASCCIAFQNLIDYIYEEQIKTKPPKAELIAIDPHAAHGSEVDRLKAKEKALQRKQEQRMARHFAIISREQTHFSEDDSKRLKTISYQLSVDIPEKQIIDDIVFDFQLRNSNMSIICCDSRIACGKVVRNELLEKHYKHGSKFLTSFPDGTTQIFYPSGNLAIIRVPNKVNGFTCIVQEDMPTNPAILAVLDSSGRSSCYHPNGNVWVYINILGGQYSDQAGNRIRAWNWSNSITSSPFVSFKPVFLALNRYIGVRILEQDKISITFLAMGQQARISVGTKVKLPNPEEIPILRYVSGDDLLLLASLIKIRRLFHKLEGCVNFPSSQVWEKLKQPSYLSSLSLKLIALCHSSGIKQDIMKTIRNIINEEI

SEQ ID NO: 7 - protein, 332 aa, Homo sapiens
MEPVGRKRSRKAAKAQLEAQVTAAQGATKEGSGIASNFPGQPTMEPVGRKRSRKAAKAQLEAQVRAAPAKKHTGKDPVRDECEERNPFTETREEDVTDEHGEREPFAEKDEHTGIHTMKLEHIAADIKKGLAAKREMIKIDKAAYRKTKNTIERALKKKQLKRQKRDYRHTRKLLNVLKEYIAEKQKDDEAEEAEAAAAAAEAAAAAEAAAAAAEVIVVEDEEEEEKEEEEEKEEEEEEGEEEGGGEEGEEGGGGGEGEETEEEEEEEEEEEEEEQIKAFQEKQKRWQQPTGVRSWRLREMKPLLEQLLKAAKDTKDNYCIISSSEESELDN

SEQ ID NO: 8 - protein, 1220 aa, Homo sapiens
MGKKQKNKSEDSTKDDIDLDALAAEIEGAGAAKEQEPQKSKGKKKKEKKKQDFDEDDILKELEELSLEAQGIKADRETVAVKPTENNEEEFTSKDKKKKGQKGKKQSFDDNDSEELEDKDSKSKKTAKPKVEMYSGSDDDDDFNKLPKKAKGKAQKSNKKWDGSEEDEDNSKKIKERSRINSSGESGDESDEFLQSRKGQKKNQKNKPGPNIESGNEDDDASFKIKTVAQKKAEKKERERKKRDEEKAKLRKLKEKEELETGKKDQSKQKESQRKFEEETVKSKVTVDTGVIPASEEKAETPTAAEDDNEGDKKKKDKKKKKGEKEEKEKEKKKGPSKATVKAMQEALAKLKEEEERQKREEEERIKRLEELEAKRKEEERLEQEKRERKKQKEKERKERLKKEGKLLTKSQREARARAEATLKLLQAQGVEVPSKDSLPKKRPIYEDKKRKKIPQQLESKEVSESMELCAAVEVMEQGVPEKEETPPPVEPEEEEDTEDAGLDDWEAMASDEETEKVEGNKVHIEVKENPEEEEEEEEEEEEDEESEEEEEEEGESEGSEGDEEDEKVSDEKDSGKTLDKKPSKEMSSDSEYDSDDDRTKEERAYDKAKRRIEKRRLEHSKNVNTEKLRAPIICVLGHVDTGKTKILDKLRHTHVQDGEAGGITQQIGATNVPLEAINEQTKMIKNFDRENVRIPGMLIIDTPGHESFSNLRNRGSSLCDIAILVVDIMHGLEPQTIESINLLKSKKCPFIVALNKIDRLYDWKKSPDSDVAATLKKQKKNTKDEFEERAKAIIVEFAQQGLNAALFYENKDPRTFVSLVPTSAHTGDGMGSLIYLLVELTQTMLSKRLAHCEELRAQVMEVKALPGMGTTIDVILINGRLKEGDTIIVPGVEGPIVTQIRGLLLPPPMKELRVKNQYEKHKEVEAAQGVKILGKDLEKTLAGLPLLVAYKEDEIPVLKDELIHELKQTLNAIKLEEKGVYVQASTLGSLEALLEFLKTSEVPYAGINIGPVHKKDVMKASVMLEHDPQYAVILAFDVRIERDAQEMADSLGVRIFSAEIIYHLFDAFTKYRQDYKKQKQEEFKHIAVFPCKIKILPQYIFNSRDPIVMGVTVEAGQVKQGTPMCVPSKNFVDIGIVTSIEINHKQVDVAKKGQEVCVKIEPIPGESPKMFGRHFEATDILVSKISRQSIDALKDWFRDEMQKSDWQLIVELKKVFEII

SEQ ID NO: 9 - protein, 774 aa, Homo sapiens
MRGPIVLHICLAFCSLLLFSVATQCLAFPKIERRREIAHVHAEKGQSDKMNTDDLENSSVTSKQTPQLVVSEDPMMMSAVPSATSLNKAFSINKETQPGQAGLMQTERPGVSTPTESGVPSAEEVFGSSQPERISPESGLAKAMLTIAITATPSLTVDEKEELLTSTNFQPIVEEITETTKGFLKYMDNQSFATESQEGVGLGHSPSSYVNTKEMLTTNPKTEKFEADTDHRTTSFPGAESTAGSEPGSLTPDKEKPSQMTADNTQAAATKQPLETSEYTLSVEPETDSLLGAPEVTVSVSTAVPAASALSDEWDDTKLESVSRIRTPKLGDNEETQVRTEMSQTAQVSHEGMEGGQPWTEAAQVALGLPEGETHTGTALLIAHGNERSPAFTDQSSFTPTSLMEDMKVSIVNLLQSTGDFTESTKENDALFFLETTVSVSVYESEADQLLGNTMKDIITQEMTTAVQEPDATLSMVTQEQVATLELIRDSGKTEEEKEDPSPVSDVPGVTQLSRRWEPLATTISTTVVPLSFEVTPTVEEQMDTVTGPNEEFTPVLGSPVTPPGIMVGEPSISPALPALEASSERRTVVPSITRVNTAASYGLDQLESEEGQEDEDEEDEEDEDEEEEDEEEDEEDKDADSLDEGLDGDTELPGFTLPGITSQEPGLEEGNMDLLEGATYQVPDALEWEQQNQGLVRSWMEKLKDKAGYMSGMLVPVGVGIAGALFILGALYSIKVMNRRRRNGFKRHKRKQREFNSMQDRVMLLADSSEDEF

SEQ ID NO: 10 - protein, 546 aa, Homo sapiens
MGAYLSQPNTVKCSGDGVGAPRLPLPYGFSAMQGWRVSMEDAHNCIPELDSETAMFSVYDGHGGEEVALYCAKYLPDIIKDQKAYKEGKLQKALEDAFLAIDAKLTTEEVIKELAQIAGRPTEDEDEKEKVADEDDVDNEEAALLHEEATMTIEELLTRYGQNCHKGPPHSKSGGGTGEEPGSQGLNGEAGPEDSTRETPSQENGPTAKAYTGFSSNSERGTEAGQVGEPGIPTGEAGPSCSSASDKLPRVAKSKFFEDSEDESDEAEEEEEDSEECSEEEDGYSSEEAENEEDEDDTEEAEEDDEEEEEEMMVPGMEGKEEPGSDSGTTAVVALIRGKQLIVANAGDSRCVVSEAGKALDMSYDHKPEDEVELARIKNAGGKVTMDGRVNGGLNLSRAIGDHFYKRNKNLPPEEQMISALPDIKVLTLTDDHEFMVIACDGIWNVMSSQEVVDFIQSKISQRDENGELRLLSSIVEELLDQCLAPDTSGDGTGCDNMTCIIICFKPRNTAELQPESGKRKLEEVLSTEGAEENGNSDKKKKAKRD

SEQ ID NO: 11 - protein, 587 aa, Homo sapiens
MASEDIAKLAETLAKTQVAGGQLSFKGKSLKLNTAEDAKDVIKEIEDFDSLEALRLEGNTVGVEAARVIAKALEKKSELKRCHWSDMFTGRLRTEIPPALISLGEGLITAGAQLVELDLSDNAFGPDGVQGFEALLKSSACFTLQELKLNNCGMGIGGGKILAAALTECHRKSSAQGKPLALKVFVAGRNRLENDGATALAEAFRVIGTLEEVHMPQNGINHPGITALAQAFAVNPLLRVINLNDNTFTEKGAVAMAETLKTLRQVEVINFGDCLVRSKGAVAIADAIRGGLPKLKELNLSFCEIKRDAALAVAEAMADKAELEKLDLNGNTLGEEGCEQLQEVLEGFNMAKVLASLSDDEDEEEEEEGEEEEEEAEEEEEEDEEEEEEEEEEEEEEPQQRGQGEKSATPSRKILDPNTGEPAPVLSSPPPADVSTFLAFPSPEKLLRLGPKSSVLIAQQTDTSDPEKVVSAFLKVSSVFKDEATVRMAVQDAVDALMQKAFNSSSFNSNTFLTRLLVHMGLLKSEDKVKAIANLYGPLMALNHMVQQDYFPKALAPLLLAFVTKPNSALESCSFARHSLLQTLYKV

SEQ ID NO: 12 - protein, 710 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 13 - protein, 1151 aa, Homo sapiens
MLRRVTVAAVCATRRKLCEAGRELAALWGIETRGRCEDSAAARPFPILAMPGRNKAKSTCSCPDLQPNGQDLGENSRVARLGADESEEEGRRGSLSNAGDPEIVKSPSDPKQYRYIKLQNGLQALLISDLSNMEGKTGNTTDDEEEEEVEEEEEDDDEDSGAEIEDDDEEGFDDEDEFDDEHDDDLDTEDNELEELEERAEARKKTTEKQSAAALCVGVGSFADPDDLPGLAHFLEHMVFMGSLKYPDENGFDAFLKKHGGSDNASTDCERTVFQFDVQRKYFKEALDRWAQFFIHPLMIRDAIDREVEAVDSEYQLARPSDANRKEMLFGSLARPGHPMGKFFWGNAETLKHEPRKNNIDTHARLREFWMRYYSSHYMTLVVQSKETLDTLEKWVTEIFSQIPNNGLPRPNFGHLTDPFDTPAFNKLYRVVPIRKIHALTITWALPPQQQHYRVKPLHYISWLVGHEGKGSILSFLRKKCWALALFGGNGETGFEQNSTYSVFSISITLTDEGYEHFYEVAYTVFQYLKMLQKLGPEKRIFEEIRKIEDNEFHYQEQTDPVEYVENMCENMQLYPLQDILTGDQLLFEYKPEVIGEALNQLVPQKANLVLLSGANEGKCDLKEKWFGTQYSIEDIENSWAELWNSNFELNPDLHLPAENKYIATDFTLKAFDCPETEYPVKIVNTPQGCLWYKKDNKFKIPKAYIRFHLISPLIQKSAANVVLFDIFVNILTHNLAEPAYEADVAQLEYKLVAGEHGLIIRVKGFNHKLPLLFQLIIDYLAEFNSTPAVFTMITEQLKKTYFNILIKPETLAKDVRLLILEYARWSMIDKYQALMDGLSLESLLSFVKEFKSQLFVEGLVQGNVTSTESMDFLKYVVDKLNFKPLEQEMPVQFQVVELPSGHHLCKVKALNKGDANSEVTVYYQSGTRSLREYTLMELLVMHMEEPCFDFLRTKQTLGYHVYPTCRNTSGILGFSVTVGTQATKYNSEVVDKKIEEFLSSFEEKIENLTEEAFNTQVTALIKLKECEDTHLGEEVDRNWNEVVTQQYLFDRLAHEIEALKSFSKSDLVNWFKAHRGPGSKMLSVHVVGYGKYELEEDGTPSSEDSNSSCEVMQLTYLPTSPLLADCIIPITDIRAFTTTLNLLPYHKIVK

SEQ ID NO: 14 - protein, 3703 aa, Homo sapiens
MEGCDSPVVSGKDNGCGIPQHQQWTELNSTHLPDKPSSMEQSTGESHGPLDSLRAPFNERLAESTASAGPPSEPASKEVTCNECSASFASLQTYMEHHCPSARPPPPLREESASDTGEEGDEESDVENLAGEIVYQPDGSAYIVESLSQLTQGGGACGSGSGSGPLPSLFLNSLPGAGGKQGDPSCAAPVYPQIINTFHIASSFGKWFEGPDQAFPNTSALAGLSPVLHSFRVFDVRHKSNKDYLNSDGSAKSSCVSKDVPNNVDLSKFDGFVLYGKRKPILMCFLCKLSFGYVRSFVTHAVHDHRMTLSEDERKILSNKNISAIIQGIGKDKEPLVSFLEPKNKNFQHPLVSTANLIGPGHSFYGKFSGIRMEGEEALPAGSAAGPEQPQAGLLTPSTLLNLGGLTSSVLKTPITSVPLGPLASSPTKSSEGKDSGAAEGEKQEVGDGDCFSEKVEPAEEEAEEEEEEEEAEEEEEEEEEEEEEEEDEGCKGLFPSELDEELEDRPHEEPGAAAGSSSKKDLALSNQSISNSPLMPNVLQTLSRGTASTSSNSASSFVVFDGANRRNRLSFNSEGVRANVAEGGRRLDFADESANKDNATAPEPNESTEGDDGGFVPHHQHAGSLCELGVGECPSGSGVECPKCDTVLGSSRSLGGHMTMMHSRNSCKTLKCPKCNWHYKYQQTLEAHMKEKHPEPGGSCVYCKSGQPHPRLARGESYTCGYKPFRCEVCNYSTTTKGNLSIHMQSDKHLNNMQNLQNGGGEQVFSHTAGAAAAAVAAAAAAANISSSCGAPSPTKPKTKPTWRCEVCDYETNVARNLRIHMTSEKHMHNMMLLQQNMTQIQHNRHLGLGSLPSPAEAELYQYYLAQNMNLPNLKMDSAASDAQFMMSGFQLDPAGPMAAMTPALVGGEIPLDMRLGGGQLVSEELMNLGESFIQTNDPSLKLFQCAVCNKFTTDNLDMLGLHMNVERSLSEDEWKAVMGDSYQCKLCRYNTQLKANFQLHCKTDKHVQKYQLVAHIKEGGKANEWRLKCVAIGNPVHLKCNACDYYTNSLEKLRLHTVNSRHEASLKLYKHLQQHESGVEGESCYYHCVLCNYSTKAKLNLIQHVRSMKHQRSESLRKLQRLQKGLPEEDEDLGQIFTIRRCPSTDPEEAIEDVEGPSETAADPEELAKDQEGGASSSQAEKELTDSPATSKRISFPGSSESPLSSKRPKTAEEIKPEQMYQCPYCKYSNADVNRLRVHAMTQHSVQPMLRCPLCQDMLNNKIHLQLHLTHLHSVAPDCVEKLIMTVTTPEMVMPSSMFLPAAVPDRDGNSNLEEAGKQPETSEDLGKNILPSASTEQSGDLKPSPADPGSVREDSGFICWKKGCNQVFKTSAALQTHFNEVHAKRPQLPVSDRHVYKYRCNQCSLAFKTIEKLQLHSQYHVIRAATMCCLCQRSFRTFQALKKHLETSHLELSEADIQQLYGGLLANGDLLAMGDPTLAEDHTIIVEEDKEEESDLEDKQSPTGSDSGSVQEDSGSEPKRALPFRKGPNFTMEKFLDPSRPYKCTVCKESFTQKNILLVHYNSVSHLHKLKRALQESATGQPEPTSSPDNKPFKCNTCNVAYSQSSTLEIHMRSVLHQTKARAAKLEAASGSSNGTGNSSSISLSSSTPSPVSTSGSNTFTTSNPSSAGIAPSSNLLSQVPTESVGMPPLGNPIGANIASPSEPKEANRKKLADMIASRQQQQQQQQQQQQQQQQQQQAQTLAQAQAQVQAHLQQELQQQAALIQSQLFNPTLLPHFPMTTETLLQLQQQQHLLFPFYIPSAEFQLNPEVSLPVTSGALTLTGTGPGLLEDLKAQVQVPQQSHQQILPQQQQNQLSIAQSHSALLQPSQHPEKKNKLVIKEKEKESQRERDSAEGGEGNTGPKETLPDALKAKEKKELAPGGGSEPSMLPPRIASDARGNATKALLENFGFELVIQYNENKQKVQKKNGKTDQGENLEKLECDSCGKLFSNILILKSHQEHVHQNYFPFKQLERFAKQYRDHYDKLYPLRPQTPEPPPPPPPPPPPPLPAAPPQPASTPAIPASAPPITSPTIAPAQPSVPLTQLSMPMELPIFSPLMMQTMPLQTLPAQLPPQLGPVEPLPADLAQLYQHQLNPTLLQQQNKRPRTRITDDQLRVLRQYFDINNSPSEEQIKEMADKSGLPQKVIKHWFRNTLFKERQRNKDSPYNFSNPPITSLEELKIDSRPPSPEPPKQEYWGSKRSSRTRFTDYQLRVLQDFFDANAYPKDDEFEQLSNLLNLPTRVIVVWFQNARQKARKNYENQGEGKDGERRELTNDRYIRTSNLNYQCKKCSLVFQRIFDLIKHQKKLCYKDEDEEGQDDSQNEDSMDAMEILTPTSSSCSTPMPSQAYSAPAPSANNTASSAFLQLTAEAEELATFNSKTEAGDEKPKLAEAPSAQPNQTQEKQGQPKPELQQQEQPEQKTNTPQQKLPQLVSLPSLPQPPPQAPPPQCPLPQSSPSPSQLSHLPLKPLHTSTPQQLANLPPQLIPYQCDQCKLAFPSFEHWQEHQQLHFLSAQNQFIHPQFLDRSLDMPFMLFDPSNPLLASQLLSGAIPQIPASSATSPSTPTSTMNTLKRKLEEKASASPGENDSGTGGEEPQRDKRLRTTITPEQLEILYQKYLLDSNPTRKMLDHIAHEVGLKKRVVQVWFQNTRARERKGQFRAVGPAQAHRRCPFCRALFKAKTALEAHIRSRHWHEAKRAGYNLTLSAMLLDCDGGLQMKGDIFDGTSFSHLPPSSSDGQGVPLSPVSKTMELSPRTLLSPSSIKVEGIEDFESPSMSSVNLNFDQTKLDNDDCSSVNTAITDTTTGDEGNADNDSATGIATETKSSSAPNEGLTKAAMMAMSEYEDRLSSGLVSPAPSFYSKEYDNEGTVDYSETSSLADPCSPSPGASGSAGKSGDSGDRPGQKRFRTQMTNLQLKVLKSCFNDYRTPTMLECEVLGNDIGLPKRVVQVWFQNARAKEKKSKLSMAKHFGINQTSYEGPKTECTLCGIKYSARLSVRDHIFSQQHISKVKDTIGSQLDKEKEYFDPATVRQLMAQQELDRIKKANEVLGLAAQQQGMFDNTPLQALNLPTAYPALQGIPPVLLPGLNSPSLPGFTPSNTALTSPKPNLMGLPSTTVPSPGLPTSGLPNKPSSASLSSPTPAQATMAMGPQQPPQQQQQQQQPQVQQPPPPPAAQPPPTPQLPLQQQQQRKDKDSEKVKEKEKAHKGKGEPLPVPKKEKGEAPTATAATISAPLPTMEYAVDPAQLQALQAALTSDPTALLTSQFLPYFVPGFSPYYAPQIPGALQSGYLQPMYGMEGLFPYSPALSQALMGLSPGSLLQQYQQYQQSLQEAIQQQQQRQLQQQQQQKVQQQQPKASQTPVPPGAPSPDKDPAKESPKPEEQKNTPREVSPLLPKLPEEPEAESKSADSLYDPFIVPKVQYKLVCRKCQAGFSDEEAARSHLKSLCFFGQSVVNLQEMVLHVPTGGGGGGSGGGGGGGGGGGGGGSYHCLACESALCGEEALSQHLESALHKHRTITRAARNAKEHPSLLPHSACFPDPSTASTSQSAAHSNDSPPPPSAAAPSSASPHASRKSWPQVVSRASAAKPPSFPPLSSSSTVTSSSCSTSGVQPSMPTDDYSEESDTDLSQKSDGPASPVEGPKDPSCPKDSGLTSVGTDTFRL

SEQ ID NO: 15 - protein, 619 aa, Homo sapiens
MANDIDELIGIPFPNHSSEVLCSLNEQRHDGLLCDVLLVVQEQEYRTHRSVLAACSKYFKKLFTAGTLASQPYVYEIDFVQPEALAAILEFAYTSTLTITAGNVKHILNAARMLEIQCIVNVCLEIMEPGGDGGEEDDKEDDDDDEDDDDEEDEEEEEEEEEDDDDDTEDFADQENLPDPQDISCHQSPSKTDHLTEKAYSDTPRDFPDSFQAGSPGHLGVIRDFSIESLLRENLYPKANIPDRRPSLSPFAPDFFPHLWPGDFGAFAQLPEQPMDSGPLDLVIKNRKIKEEEKEELPPPPPPPFPNDFFKDMFPDLPGGPLGPIKAENDYGAYLNFLSATHLGGLFPPWPLVEERKLKPKASQQCPICHKVIMGAGKLPRHMRTHTGEKPYMCTICEVRFTRQDKLKIHMRKHTGERPYLCIHCNAKFVHNYDLKNHMRIHTGVRPYQCEFCYKSFTRSDHLHRHIKRQSCRMARPRRGRKPAAWRAASLLFGPGGPAPDKAAFVMPPALGEVGGHLGGAAVCLPGPSPAKHFLAAPKGALSLQELERQFEETQMKLFGRAQLEAERNAGGLLAFALAENVAAARPYFPLPDPWAAGLAGLPGLAGLNHVASMSEANN

SEQ ID NO: 16 - protein, 1124 aa, Homo sapiens
MADGPRCKRRKQANPRRNNVTNYNTVVETNSDSDDEDKLHIVEEESVTDAADCEGVPEDDLPTDQTVLPGRSSEREGNAKNCWEDDRKEGQEILGPEAQADEAGCTVKDDECESDAENEQNHDPNVEEFLQQQDTAVIFPEAPEEDQRQGTPEASGHDENGTPDAFSQLLTCPYCDRGYKRFTSLKEHIKYRHEKNEDNFSCSLCSYTFAYRTQLERHMTSHKSGRDQRHVTQSGCNRKFKCTECGKAFKYKHHLKEHLRIHSGEKPYECPNCKKRFSHSGSYSSHISSKKCISLIPVNGRPRTGLKTSQCSSPSLSASPGSPTRPQIRQKIENKPLQEQLSVNQIKTEPVDYEFKPIVVASGINCSTPLQNGVFTGGGPLQATSSPQGMVQAVVLPTVGLVSPISINLSDIQNVLKVAVDGNVIRQVLENNQANLASKEQETINASPIQQGGHSVISAISLPLVDQDGTTKIIINYSLEQPSQLQVVPQNLKKENPVATNSCKSEKLPEDLTVKSEKDKSFEGGVNDSTCLLCDDCPGDINALPELKHYDLKQPTQPPPLPAAEAEKPESSVSSATGDGNLSPSQPPLKNLLSLLKAYYALNAQPSAEELSKIADSVNLPLDVVKKWFEKMQAGQISVQSSEPSSPEPGKVNIPAKNNDQPQSANANEPQDSTVNLQSPLKMTNSPVLPVGSTTNGSRSSTPSPSPLNLSSSRNTQGYLYTAEGAQEEPQVEPLDLSLPKQQGELLERSTITSVYQNSVYSVQEEPLNLSCAKKEPQKDSCVTDSEPVVNVIPPSANPINIAIPTVTAQLPTIVAIADQNSVPCLRALAANKQTILIPQVAYTYSTTVSPAVQEPPLKVIQPNGNQDERQDTSSEGVSNVEDQNDSDSTPPKKKMRKTENGMYACDLCDKIFQKSSSLLRHKYEHTGKRPHECGICKKAFKHKHHLIEHMRLHSGEKPYQCDKCGKRFSHSGSYSQHMNHRYSYCKREAEERDSTEQEEAGPEILSNEHVGARASPSQGDSDERESLTREEDEDSEKEEEEEDKEMEELQEEKECEKPQGDEEEEEEEEEVEEEEVEEAENEGEEAKTEGLMKDDRAESQASSLGQKVGESSEQVSEEKTNEA

SEQ ID NO: 17 - protein, 727 aa, Homo sapiens
MAADSREEKDGELNVLDDILTEVPEQDDELYNPESEQDKNEKKGSKRKSDRMESTDTKRQKPSVHSRQLVSKPLSSSVSNNKRIVSTKGKSATEYKNEEYQRSERNKRLDADRKIRLSSSASREPYKNQPEKTCVRKRDPERRAKSPTPDGSERIGLEVDRRASRSSQSSKEEVNSEEYGSDHETGSSGSSDEQGNNTENEEEGVEEDVEEDEEVEEDAEEDEEVDEDGEEEEEEEEEEEEEEEEEEEEYEQDERDQKEEGNDYDTRSEASDSGSESVSFTDGSVRSGSGTDGSDEKKKERKRARGISPIVFDRSGSSASESYAGSEKKHEKLSSSVRAVRKDQTSKLKYVLQDARFFLIKSNNHENVSLAKAKGVWSTLPVNEKKLNLAFRSARSVILIFSVRESGKFQGFARLSSESHHGGSPIHWVLPAGMSAKMLGGVFKIDWICRRELPFTKSAHLTNPWNEHKPVKIGRDGQEIELECGTQLCLLFPPDESIDLYQVIHKMRHKRRMHSQPRSRGRPSRREPVRDVGRRRPEDYDIHNSRKKPRIDYPPEFHQRPGYLKDPRYQEVDRRFSGVRRDVFLNGSYNDYVREFHNMGPPPPWQGMPPYPGMEQPPHHPYYQHHAPPPQAHPPYSGHHPVPHEARYRDKRVHDYDMRVDDFLRRTQAVVSGRRSRPRERDRERERDRPRDNRRDRERDRGRDRERERERLCDRDRDRGERGRYRR

SEQ ID NO: 18 - protein, 747 aa, Homo sapiens
MGRLNEQRLFQPDLCDVDLVLVPQRSVFPAHKGVLAAYSQFFHSLFTQNKQLQRVELSLEALAPGGLQQILNFIYTSKLLVNAANVHEVLSAASLLQMADIAASCQELLDARSLGPPGPGTVALAQPAASCTPAAPPYYCDIKQEADTPGLPKIYAREGPDPYSVRVEDGAGTAGGTVPATIGPAQPFFKEEKEGGVEEAGGPPASLCKLEGGEELEEELGGSGTYSRREQSQIIVEVNLNNQTLHVSTGPEGKPGAGPSPATVVLGREDGLQRHSDEEEEDDEEEEEEEEEEEGGGSGREEEEEEEGGSQGEEEEEEEDGHSEQEEEEEEEEEEGPSEQDQESSEEEEGEEGEAGGKQGPRGSRSSRADPPPHSHMATRSRENARRRGTPEPEEAGRRGGKRPKPPPGVASASARGPPATDGLGAKVKLEEKQHHPCQKCPRVFNNRWYLEKHMNVTHSRMQICDQCGKRFLLESELLLHRQTDCERNIQCVTCGKAFKKLWSLHEHNKIVHGYAEKKFSCEICEKKFYTMAHVRKHMVAHTKDMPFTCETCGKSFKRSMSLKVHSLQHSGEKPFRCENCNERFQYKYQLRSHMSIHIGHKQFMCQWCGKDFNMKQYFDEHMKTHTGEKPYICEICGKSFTSRPNMKRHRRTHTGEKPYPCDVCGQRFRFSNMLKAHKEKCFRVSHTLAGDGVPAAPGLPPTQPQAHALPLLPGLPQTLPPPPHLPPPPPLFPTTASPGGRMNANN

SEQ ID NO: 19 - protein, 1321 aa, Homo sapiens
MQCLAAALKDETNMSGGGEQADILPANYVVKDRWKVLKKIGGGGFGEIYEAMDLLTRENVALKVESAQQPKQVLKMEVAVLKKLQGKDHVCRFIGCGRNEKFNYVVMQLQGRNLADLRRSQPRGTFTLSTTLRLGKQILESIEAIHSVGFLHRDIKPSNFAMGRLPSTYRKCYMLDFGLARQYTNTTGDVRPPRNVAGFRGTVRYASVNAHKNREMGRHDDLWSLFYMLVEFAVGQLPWRKIKDKEQVGMIKEKYEHRMLLKHMPSEFHLFLDHIASLDYFTKPDYQLIMSVFENSMKERGIAENEAFDWEKAGTDALLSTSTSTPPQQNTRQTAAMFGVVNVTPVPGDLLRENTEDVLQGEHLSDQENAPPILPGRPSEGLGPSPHLVPHPGGPEAEVWEETDVNRNKLRINIGKSPCVEEEQSRGMGVPSSPVRAPPDSPTTPVRSLRYRRVNSPESERLSTADGRVELPERRSRMDLPGSPSRQACSSQPAQMLSVDTGHADRQASGRMDVSASVEQEALSNAFRSVPLAEEEDFDSKEWVIIDKETELKDFPPGAEPSTSGTTDEEPEELRPLPEEGEERRRLGAEPTVRPRGRSMQALAEEDLQHLPPQPLPPQLSQGDGRSETSQPPTPGSPSHSPLHSGPRPRRRESDPTGPQRQVFSVAPPFEVNGLPRAVPLSLPYQDFKRDLSDYRERARLLNRVRRVGFSHMLLTTPQVPLAPVQPQANGKEEEEEEEEDEEEEEEDEEEEEEEEEEEEEEEEEEEEEEEAAAAVALGEVLGPRSGSSSEGSERSTDRSQEGAPSTLLADDQKESRGRASMADGDLEPEEGSKTLVLVSPGDMKKSPVTAELAPDPDLGTLAALTPQHERPQPTGSQLDVSEPGTLSSVLKSEPKPPGPGAGLGAGTVTTGVGGVAVTSSPFTKVERTFVHIAEKTHLNVMSSGGQALRSEEFSAGGELGLELASDGGAVEEGARAPLENGLALSGLNGAEIEGSALSGAPRETPSEMATNSLPNGPALADGPAPVSPLEPSPEKVATISPRRHAMPGSRPRSRIPVLLSEEDTGSEPSGSLSAKERWSKRARPQQDLARLVMEKRQGRLLLRLASGASSSSSEEQRRASETLSGTGSEEDTPASEPAAALPRKSGRAAATRSRIPRPIGLRMPMPVAAQQPASRSHGAAPALDTAITSRLQLQTPPGSATAADLRPKQPPGRGLGPGRAQAGARPPAPRSPRLPASTSAARNASASPRSQSLSRRESPSPSHQARPGVPPPRGVPPARAQPDGTPSPGGSKKGPRGKLQAQRATTKGRAGGAEGRAGAR

SEQ ID NO: 20 - protein, 2073 aa, Homo sapiens
MVKLANPLYTEWILEAIQKIKKQKQRPSEERICHAVSTSHGLDKKTVSEQLELSVQDGSVLKVTNKGLASYKDPDNPGRFSSVKPGTFPKSAKGSRGSCNDLRNVDWNKLLRRAIEGLEEPNGSSLKNIEKYLRSQSDLTSTTNNPAFQQRLRLGAKRAVNNGRLLKDGPQYRVNYGSLDGKGAPQYPSAFPSSLPPVSLLPHEKDQPRADPIPICSFCLGTKESNREKKPEELLSCADCGSSGHPSCLKFCPELTTNVKALRWQCIECKTCSACRVQGRNADNMLFCDSCDRGFHMECCDPPLSRMPKGMWICQVCRPKKKGRKLLHEKAAQIKRRYAKPIGRPKNKLKQRLLSVTSDEGSMNAFTGRGSPGRGQKTKVCTTPSSGHAASGKDSSSRLAVTDPTRPGATTKITTTSTYISASTLKVNKKTKGLIDGLTKFFTPSPDGRRSRGEIIDFSKHYRPRKKVSQKQSCTSHVLATGTTQKLKPPPSSLPPPTPISGQSPSSQKSSTATSSPSPQSSSSQCSVPSLSSLTTNSQLKALFDGLSHIYTTQGQSRKKGHPSYAPPKRMRRKTELSSTAKSKAHFFGKRDIRSRFISHSSSSSWGMARGSIFKAIAHFKRTTFLKKHRMLGRLKYKVTPQMGTPSPGKGSLTDGRIKPDQDDDTEIKINIKQESADVNVIGNKDVVTEEDLDVFKQAQELSWEKIECESGVEDCGRYPSVIEFGKYEIQTWYSSPYPQEYARLPKLYLCEFCLKYMKSKNILLRHSKKCGWFHPPANEIYRRKDLSVFEVDGNMSKIYCQNLCLLAKLFLDHKTLYYDVEPFLFYVLTKNDEKGCHLVGYFSKEKLCQQKYNVSCIMIMPQHQRQGFGRFLIDFSYLLSRREGQAGSPEKPLSDLGRLSYLAYWKSVILEYLYHHHERHISIKAISRATGMCPHDIATTLQHLHMIDKRDGRFVIIRREKLILSHMEKLKTCSRANELDPDSLRWTPILISNAAVSEEEREAEKEAERLMEQASCWEKEEQEILSTRANSRQSPAKVQSKNKYLHSPESRPVTGERGQLLELSKESSEEEEEEEDEEEEEEEEEEEEDEEEEEEEEEEEEEENIQSSPPRLTKPQSVAIKRKRPFVLKKKRGRKRRRINSSVTTETISETTEVLNEPFDNSDEERPMPQLEPTCEIEVEEDGRKPVLRKAFQHQPGKKRQTEEEEGKDNHCFKNADPCRNNMNDDSSNLKEGSKDNPEPLKCKQVWPKGTKRGLSKWRQNKERKTGFKLNLYTPPETPMEPDEQVTVEEQKETSEGKTSPSPIRIEEEVKETGEALLPQEENRREETCAPVSPNTSPGEKPEDDLIKPEEEEEEEEEEEEEEEEEEGEEEEGGGNVEKDPDGAKSQEKEEPEISTEKEDSARLDDHEEEEEEDEEPSHNEDHDADDEDDSHMESAEVEKEELPRESFKEVLENQETFLDLNVQPGHSNPEVLMDCGVDLTASCNSEPKELAGDPEAVPESDEEPPPGEQAQKQDQKNSKEVDTEFKEGNPATMEIDSETVQAVQSLTQESSEQDDTFQDCAETQEACRSLQNYTRADQSPQIATTLDDCQQSDHSSPVSSVHSHPGQSVRSVNSPSVPALENSYAQISPDQSAISVPSLQNMETSPMMDVPSVSDHSQQVVDSGFSDLGSIESTTENYENPSSYDSTMGGSICGNGSSQNSCSYSNLTSSSLTQSSCAVTQQMSNISGSCSMLQQTSISSPPTCSVKSPQGCVVERPPSSSQQLAQCSMAANFTPPMQLAEIPETSNANIGLYERMGQSDFGAGHYPQPSATFSLAKLQQLTNTLIDHSLPYSHSAAVTSYANSASLSTPLSNTGLVQLSQSPHSVPGGPQAQATMTPPPNLTPPPMNLPPPLLQRNMAASNIGISHSQRLQTQIASKGHISMRTKSASLSPAAATHQSQIYGRSQTVAMQGPARTLTMQRGMNMSVNLMPAPAYNVNSVNMNMNTLNAMNGYSMSQPMMNSGYHSNHGYMNQTPQYPMQMQMGMMGTQPYAQQPMQTPPHGNMMYTAPGHHGYMNTGMSKQSLNGSYMRR

SEQ ID NO: 21 - protein, 102 aa, Homo sapiens
MSEKSVEAAAELSAKDLKEKKEKVEEKASRKERKKEVVEEEENGAEEEEEETAEDGEEEDEGEEEDEEEEEEDDEGPALKRAAEEEDEADPKRQKTENGASA

SEQ ID NO: 22 - protein, 102 aa, Homo sapiens; identical to SEQ ID NO: 21

SEQ ID NO: 23 - protein, 539 aa, Homo sapiens
MATSAPLRSLEEEVTCSICLDYLRDPVTIDCGHVFCRSCTTDVRPISGSRPVCPLCKKPFKKENIRPVWQLASLVENIERLKVDKGRQPGEVTREQQDAKLCERHREKLHYYCEDDGKLLCVMCRESREHRPHTAVLMEKAAQPHREKILNHLSTLRRDRDKIQGFQAKGEADILAALKKLQDQRQYIVAEFEQGHQFLREREEHLLEQLAKLEQELTEGREKFKSRGVGELARLALVISELEGKAQQPAAELMQDTRDFLNRYPRKKFWVGKPIARVVKKKTGEFSDKLLSLQRGLREFQGKLLRDLEYKTVSVTLDPQSASGYLQLSEDWKCVTYTSLYKSAYLHPQQFDCEPGVLGSKGFTWGKVYWEVEVEREGWSEDEEEGDEEEEGEEEEEEEEAGYGDGYDDWETDEDEESLGDEEEEEEEEEEEVLESCMVGVARDSVKRKGDLSLRPEDGVWALRLSSSGIWANTSPEAELFPALRPRRVGIALDYEGGTVTFTNAESQELIYTFTATFTRRLVPFLWLKWPGTRLLLRP

SEQ ID NO: 24 - protein, 5038 aa, Homo sapiens
MGDAEGEDEVQFLRTDDEVVLQCSATVLKEQLKLCLAAEGFGNRLCFLEPTSNAQNVPPDLAICCFVLEQSLSVRALQEMLANTVEAGVESSQGGGHRTLLYGHAILLRHAHSRMYLSCLTTSRSMTDKLAFDVGLQEDATGEACWWTMHPASKQRSEGEKVRVGDDIILVSVSSERYLHLSTASGELQVDASFMQTLWNMNPICSRCEEGFVTGGHVLRLFHGHMDECLTISPADSDDQRRLVYYEGGAVCTHARSLWRLEPLRISWSGSHLRWGQPLRVRHVTTGQYLALTEDQGLVVVDASKAHTKATSFCFRISKEKLDVAPKRDVEGMGPPEIKYGESLCFVQHVASGLWLTYAAPDPKALRLGVLKKKAMLHQEGHMDDALSLTRCQQEESQAARMIHSTNGLYNQFIKSLDSFSGKPRGSGPPAGTALPIEGVILSLQDLIIYFEPPSEDLQHEEKQSKLRSLRNRQSLFQEEGMLSMVLNCIDRLNVYTTAAHFAEFAGEEAAESWKEIVNLLYELLASLIRGNRSNCALFSTNLDWLVSKLDRLEASSGILEVLYCVLIESPEVLNIIQENHIKSIISLLDKHGRNHKVLDVLCSLCVCNGVAVRSNQDLITENLLPGRELLLQTNLINYVTSIRPNIFVGRAEGTTQYSKWYFEVMVDEVTPFLTAQATHLRVGWALTEGYTPYPGAGEGWGGNGVGDDLYSYGFDGLHLWTGHVARPVTSPGQHLLAPEDVISCCLDLSVPSISFRINGCPVQGVFESFNLDGLFFPVVSFSAGVKVRFLLGGRHGEFKFLPPPGYAPCHEAVLPRERLHLEPIKEYRREGPRGPHLVGPSRCLSHTDFVPCPVDTVQIVLPPHLERIREKLAENIHELWALTRIEQGWTYGPVRDDNKRLHPCLVDFHSLPEPERNYNLQMSGETLKTLLALGCHVGMADEKAEDNLKKTKLPKTYMMSNGYKPAPLDLSHVRLTPAQTTLVDRLAENGHNVWARDRVGQGWSYSAVQDIPARRNPRLVPYRLLDEATKRSNRDSLCQAVRTLLGYGYNIEPPDQEPSQVENQSRCDRVRIFRAEKSYTVQSGRWYFEFEAVTTGEMRVGWARPELRPDVELGADELAYVFNGHRGQRWHLGSEPFGRPWQPGDVVGCMIDLTENTIIFTLNGEVLMSDSGSETAFREIEIGDGFLPVCSLGPGQVGHLNLGQDVSSLRFFAICGLQEGFEPFAINMQRPVTTWFSKGLPQFEPVPLEHPHYEVSRVDGTVDTPPCLRLTHRTWGSQNSLVEMLFLRLSLPVQFHQHFRCTAGATPLAPPGLQPPAEDEARAAEPDPDYENLRRSAGGWSEAENGKEGTAKEGAPGGTPQAGGEAQPARAENEKDATTEKNKKRGFLFKAKKVAMMTQPPATPTLPRLPHDVVPADNRDDPEIILNTTTYYYSVRVFAGQEPSCVWAGWVTPDYHQHDMSFDLSKVRVVTVTMGDEQGNVHSSLKCSNCYMVWGGDFVSPGQQGRISHTDLVIGCLVDLATGLMTFTANGKESNTFFQVEPNTKLFPAVFVLPTHQNVIQFELGKQKNIMPLSAAMFQSERKNPAPQCPPRLEMQMLMPVSWSRMPNHFLQVETRRAGERLGWAVQCQEPLTMMALHIPEENRCMDILELSERLDLQRFHSHTLRLYRAVCALGNNRVAHALCSHVDQAQLLHALEDAHLPGPLRAGYYDLLISIHLESACRSRRSMLSEYIVPLTPETRAITLFPPGRSTENGHPRHGLPGVGVTTSLRPPHHFSPPCFVAALPAAGAAEAPARLSPAIPLEALRDKALRMLGEAVRDGGQHARDPVGGSVEFQFVPVLKLVSTLLVMGIFGDEDVKQILKMIEPEVFTEEEEEEDEEEEGEEEDEEEKEEDEEETAQEKEDEEKEEEEAAEGEKEEGLEEGLLQMKLPESVKLQMCHLLEYFCDQELQHRVESLAAFAERYVDKLQANQRSRYGLLIKAFSMTAAETARRTREFRSPPQEQINMLLQFKDGTDEEDCPLPEEIRQDLLDFHQDLLAHCGIQLDGEEEEPEEETTLGSRLMSLLEKVRLVKKKEEKPEEERSAEESKPRSLQELVSHMVVRWAQEDFVQSPELVRAMFSLLHRQYDGLGELLRALPRAYTISPSSVEDTMSLLECLGQIRSLLIVQMGPQEENLMIQSIGNIMNNKVFYQHPNLMRALGMHETVMEVMVNVLGGGESKEIRFPKMVTSCCRFLCYFCRISRQNQRSMFDHLSYLLENSGIGLGMQGSTPLDVAAASVIDNNELALALQEQDLEKVVSYLAGCGLQSCPMLVAKGYPDIGWNPCGGERYLDFLRFAVFVNGESVEENANVVVRLLIRKPECFGPALRGEGGSGLLAAIEEAIRISEDPARDGPGIRRDRRREHFGEEPPEENRVHLGHAIMSFYAALIDLLGRCAPEMHLIQAGKGEALRIRAILRSLVPLEDLVGIISLPLQIPTLGKDGALVQPKMSASFVPDHKASMVLFLDRVYGIENQDFLLHVLDVGFLPDMRAAASLDTATFSTTEMALALNRYLCLAVLPLITKCAPLFAGTEHRAIMVDSMLHTVYRLSRGRSLTKAQRDVIEDCLMSLCRYIRPSMLQHLLRRLVFDVPILNEFAKMPLKLLTNHYERCWKYYCLPTGWANFGVTSEEELHLTRKLFWGIFDSLAHKKYDPELYRMAMPCLCAIAGALPPDYVDASYSSKAEKKATVDAEGNFDPRPVETLNVIIPEKLDSFINKFAEYTHEKWAFDKIQNNWSYGENIDEELKTHPMLRPYKTFSEKDKEIYRWPIKESLKAMIAWEWTIEKAREGEEEKTEKKKTRKISQSAQTYDPREGYNPQPPDLSAVTLSRELQAMAEQLAENYHNTWGRKKKQELEAKGGGTHPLLVPYDTLTAKEKARDREKAQELLKFLQMNGYAVTRGLKDMELDSSSIEKRFAFGFLQQLLRWMDISQEFIAHLEAVVSSGRVEKSPHEQEIKFFAKILLPLINQYFTNHCLYFLSTPAKVLGSGGHASNKEKEMITSLFCKLAALVRHRVSLFGTDAPAVVNCLHILARSLDARTVMKSGPEIVKAGLRSFFESASEDIEKMVENLRLGKVSQARTQVKGVGQNLTYTTVALLPVLTTLFQHIAQHQFGDDVILDDVQVSCYRTLCSIYSLGTTKNTYVEKLRPALGECLARLAAAMPVAFLEPQLNEYNACSVYTTKSPRERAILGLPNSVEEMCPDIPVLERLMADIGGLAESGARYTEMPHVIEITLPMLCSYLPRWWERGPEAPPSALPAGAPPPCTAVTSDHLNSLLGNILRIIVNNLGIDEASWMKRLAVFAQPIVSRARPELLQSHFIPTIGRLRKRAGKVVSEEEQLRLEAKAEAQEGELLVRDEFSVLCRDLYALYPLLIRYVDNNRAQWLTEPNPSAEELFRMVGEIFIYWSKSHNFKREEQNFVVQNEINNMSFLTADNKSKMAKAGDIQSGGSDQERTKKKRRGDRYSVQTSLIVATLKKMLPIGLNMCAPTDQDLITLAKTRYALKDTDEEVREFLHNNLHLQGKVEGSPSLRWQMALYRGVPGREEDADDPEKIVRRVQEVSAVLYYLDQTEHPYKSKKAVWHKLLSKQRRRAVVACFRMTPLYNLPTHRACNMFLESYKAAWILTEDHSFEDRMIDDLSKAGEQEEEEEEVEEKKPDPLHQLVLHFSRTALTEKSKLDEDYLYMAYADIMAKSCHLEEGGENGEAEEEVEVSFEEKQMEKQRLLYQQARLHTRGAAEMVLQMISACKGETGAMVSSTLKLGISILNGGNAEVQQKMLDYLKDKKEVGFFQSIQALMQTCSVLDLNAFERQNKAEGLGMVNEDGTVINRQNGEKVMADDEFTQDLFRFLQLLCEGHNNDFQNYLRTQTGNTTTINIIICTVDYLLRLQESISDFYWYYSGKDVIEEQGKRNFSKAMSVAKQVFNSLTEYIQGPCTGNQQSLAHSRLWDAVVGFLHVFAHMMMKLAQDSSQIELLKELLDLQKDMVVMLLSLLEGNVVNGMIARQMVDMLVESSSNVEMILKFFDMFLKLKDIVGSEAFQDYVTDPRGLISKKDFQKAMDSQKQFSGPEIQFLLSCSEADENEMINCEEFANRFQEPARDIGFNVAVLLTNLSEHVPHDPRLHNFLELAESILEYFRPYLGRIEIMGASRRIERIYFEISETNRAQWEMPQVKESKRQFIFDVVNEGGEAEKMELFVSFCEDTIFEMQIAAQISEPEGEPETDEDEGAGAAEAGAEGAEEGAAGLEGTAATAAAGATARVVAAAGRALRGLSYRSLRRRVRRLRRLTAREAATAVAALLWAAVTRAGAAGAGAAAGALGLLWGSLFGGGLVEGAKKVTVTELLAGMPDPTSDEVHGEQPAGPGGDADGEGASEGAGDAAEGAGDEEEAVHEAGPGGADGAVAVTDGGPFRPEGAGGLGDMGDTTPAEPPTPEGSPILKRKLGVDGVEEELPPEPEPEPEPELEPEKADAENGEKEEVPEPTPEPPKKQAPPSPPPKKEEAGGEFWGELEVQRVKFLNYLSRNFYTLRFLALFLAFAINFILLFYKVSDSPPGEDDMEGSAAGDVSGAGSGGSSGWGLGAGEEAEGDEDENMVYYFLEESTGYMEPALRCLSLLHTLVAFLCIIGYNCLKVPLVIFKREKELARKLEFDGLYITEQPEDDDVKGQWDRLVLNTPSFPSNYWDKFVKRKVLDKHGDIYGRERIAELLGMDLATLEITAHNERKPNPPPGLLTWLMSIDVKYQIWKFGVIFTDNSFLYLGWYMVMSLLGHYNNFFFAAHLLDIAMGVKTLRTILSSVTHNGKQLVMTVGLLAVVVYLYTVVAFNFFRKFYNKSEDEDEPDMKCDDMMTCYLFHMYVGVRAGGGIGDEIEDPAGDEYELYRVVFDITFFFFVIVILLAIIQGLIIDAFGELRDQQEQVKEDMETKCFICGIGSDYFDTTPHGFETHTLEEHNLANYMFFLMYLINKDETEHTGQESYVWKMYQERCWDFFPAGDCFRKQYEDQLS

SEQ ID NO: 25 - protein, 302 aa, Homo sapiens
MVWFLDFPNSMAPKRQSPLPLQKKKPRPPPALGLEETSASAGLPKKGEKEQQEAIEHIDEVQNEIDRLNEQDSEEILKVEQKYNKLRQPFFQKRSELIAKIPNFGVTTFVNHPQVSSLLGEEDEEALHYLTKVEVTEFEDIKSGYRIDFYFDENPYFENKVFSKEFHLNESGDPSSKSTKIKWKSGKDVTKRSSQTQNKASRKRQHEEPESFFTWFTDHSDAGADELEEVIKDDIWPNPLQYYLVPDMDDEEGGEDDDDDDDDGDEGEEELEDIDEGDEDEGEEDEDDDEGEEGEEDEGEDD

SEQ ID NO: 26 - protein, 290 aa, Homo sapiens
MAPKRQSPLPPQKKKPRPPPALGPEETSASAGLPKKGEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 27 - protein, 1339 aa, Homo sapiens
MTGEVGSEVHLEINDPNVISQEEADSPSDSGQGSYETIGPLSEGDSDEEIFVSKKLKNRKVLQDSDSETEDTNASPEKTTYDSAEEENKENLYAGKNTKIKRIYKTVADSDESYMEKSLYQENLEAQVKPCLELSLQSGNSTDFTTDRKSSKKHIHDKEGTAGKAKVKSKRRLEKEERKMEKIRQLKKKETKNQEDDVEQPFNDSGCLLVDKDLFETGLEDENNSPLEDEESLESIRAAVKNKVKKHKKKEPSLESGVHSFEEGSELSKGTTRKERKAARLSKEALKQLHSETQRLIRESALNLPYHMPENKTIHDFFKRKPRPTCHGNAMALLKSSKYQSSHHKEIIDTANTTEMNSDHHSKGSEQTTGAENEVETNALPVVSKETQIITGSDESCRKDLVKNEELEIQEKQKQSDIRPSPGDSSVLQQESNFLGNNHSEECQVGGLVAFEPHALEGEGPQNPEETDEKVEEPEQQNKSSAVGPPEKVRRFTLDRLKQLGVDVSIKPRLGADEDSFVILEPETNRELEALKQRFWKHANPAAKPRAGQTVNVNVIVKDMGTDGKEELKADVVPVTLAPKKLDGASHTKPGEKLQVLKAKLQEAMKLRRFEERQKRQALFKLDNEDGFEEEEEEEEEMTDESEEDGEEKVEKEEKEEELEEEEEKEEEEEEEGNQETAEFLLSSEEIETKDEKEMDKENNDGSSEIGKAVGFLSVPKSLSSDSTLLLFKDSSSKMGYFPTEEKSETDENSGKQPSKLDEDDSCSLLTKESSHNSSFELIGSTIPSYQPCNRQTGRGTSFFPTAGGFRSPSPGLFRASLVSSASKSSGKLSEPSLPIEDSQDLYNASPEPKTLFLGAGDFQFCLEDDTQSQLLDADGFLNVRNHRNQYQALKPRLPLASMDENAMDANMDELLDLCTGKFTSQAEKHLPRKSDKKENMEELLNLCSGKFTSQDASTPASSELNKQEKESSMGDPMEEALALCSGSFPTDKEEEDEEEEFGDFRLVSNDNEFDSDEDEHSDSGNDLALEDHEDDDEEELLKRSEKLKRQMRLRKYLEDEAEVSGSDVGSEDEYDGEEIDEYEEDVIDEVLPSDEELQSQIKKIHMKTMLDDDKRQLRLYQERYLADGDLHSDGPGRMRKFRWKNIDDASQMDLFHRDSDDDQTEEQLDESEARWRKERIEREQWLRDMAQQGKITAEEEEEIGEDSQFMILAKKVTAKALQKNASRPMVIQESKSLLRNPFEAIRPGSAQQVKTGSLLNQPKAVLQKLAALSDHNPSAPRNSRNFVFHTLSPVKAEAAKESSKSQVKKRGPSFMTSPSPKHLKTDDSTSGLTRSIFKYLES

SEQ ID NO: 28 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 29 - protein, 3046 aa, Homo sapiens
MRGRRGRPPKQPAAPAAERCAPAPPPPPPPPTSGPIGGLRSRHRGSSRGRWAAAQAEVAPKTRLSSPRGGSSSRRKPPPPPPAPPSTSAPGRGGRGGGGGRTGGGGGGGHLARTTAARRAVNKVVYDDHESEEEEEEEDMVSEEEEEEDGDAEETQDSEDDEEDEMEEDDDDSDYPEEMEDDDDDASYCTESSFRSHSTYSSTPGRRKPRVHRPRSPILEEKDIPPLEFPKSSEDLMVPNEHIMNVIAIYEVLRNFGTVLRLSPFRFEDFCAALVSQEQCTLMAEMHVVLLKAVLREEDTSNTTFGPADLKDSVNSTLYFIDGMTWPEVLRVYCESDKEYHHVLPYQEAEDYPYGPVENKIKVLQFLVDQFLTTNIAREELMSEGVIQYDDHCRVCHKLGDLLCCETCSAVYHLECVKPPLEEVPEDEWQCEVCVAHKVPGVTDCVAEIQKNKPYIRHEPIGYDRSRRKYWFLNRRLIIEEDTENENEKKIWYYSTKVQLAELIDCLDKDYWEAELCKILEEMREEIHRHMDITEDLTNKARGSNKSFLAAANEEILESIRAKKGDIDNVKSPEETEKDKNETENDSKDAEKNREEFEDQSLEKDSDDKTPDDDPEQGKSEEPTEVGDKGNSVSANLGDNTTNATSEETSPSEGRSPVGCLSETPDSSNMAEKKVASELPQDVPEEPNKTCESSNTSATTTSIQPNLENSNSSSELNSSQSESAKAADDPENGERESHTPVSIQEEIVGDFKSEKSNGELSESPGAGKGASGSTRIITRLRNPDSKLSQLKSQQVAAAAHEANKLFKEGKEVLVVNSQGEISRLSTKKEVIMKGNINNYFKLGQEGKYRVYHNQYSTNSFALNKHQHREDHDKRRHLAHKFCLTPAGEFKWNGSVHGSKVLTISTLRLTITQLENNIPSSFLHPNWASHRANWIKAVQMCSKPREFALALAILECAVKPVVMLPIWRESLGHTRLHRMTSIEREEKEKVKKKEKKQEEEETMQQATWVKYTFPVKHQVWKQKGEEYRVTGYGGWSWISKTHVYRFVPKLPGNTNVNYRKSLEGTKNNMDENMDESDKRKCSRSPKKIKIEPDSEKDEVKGSDAAKGADQNEMDISKITEKKDQDVKELLDSDSDKPCKEEPMEVDDDMKTESHVNCQESSQVDVVNVSEGFHLRTSYKKKTKSSKLDGLLERRIKQFTLEEKQRLEKIKLEGGIKGIGKTSTNSSKNLSESPVITKAKEGCQSDSMRQEQSPNANNDQPEDLIQGCSESDSSVLRMSDPSHTTNKLYPKDRVLDDVSIRSPETKCPKQNSIENDIEEKVSDLASRGQEPSKSKTKGNDFFIDDSKLASADDIGTLICKNKKPLIQEESDTIVSSSKSALHSSVPKSTNDRDATPLSRAMDFEGKLGCDSESNSTLENSSDTVSIQDSSEEDMIVQNSNESISEQFRTREQDVEVLEPLKCELVSGESTGNCEDRLPVKGTEANGKKPSQQKKLEERPVNKCSDQIKLKNTTDKKNNENRESEKKGQRTSTFQINGKDNKPKIYLKGECLKEISESRVVSGNVEPKVNNINKIIPENDIKSLTVKESAIRPFINGDVIMEDFNERNSSETKSHLLSSSDAEGNYRDSLETLPSTKESDSTQTTTPSASCPESNSVNQVEDMEIETSEVKKVTSSPITSEEESNLSNDFIDENGLPINKNENVNGESKRKTVITEVTTMTSTVATESKTVIKVEKGDKQTVVSSTENCAKSTVTTTTTTVTKLSTPSTGGSVDIISVKEQSKTVVTTTVTDSLTTTGGTLVTSMTVSKEYSTRDKVKLMKFSRPKKTRSGTALPSYRKFVTKSSKKSIFVLPNDDLKKLARKGGIREVPYFNYNAKPALDIWPYPSPRPTFGITWRYRLQTVKSLAGVSLMLRLLWASLRWDDMAAKAPPGGGTTRTETSETEITTTEIIKRRDVGPYGIRSEYCIRKIICPIGVPETPKETPTPQRKGLRSSALRPKRPETPKQTGPVIIETWVAEEELELWEIRAFAERVEKEKAQAVEQQAKKRLEQQKPTVIATSTTSPTSSTTSTISPAQKVMVAPISGSVTTGTKMVLTTKVGSPATVTFQQNKNFHQTFATWVKQGQSNSGVVQVQQKVLGIIPSSTGTSQQTFTSFQPRTATVTIRPNTSGSGGTTSNSQVITGPQIRPGMTVIRTPLQQSTLGKAIIRTPVMVQPGAPQQVMTQIIRGQPVSTAVSAPNTVSSTPGQKSLTSATSTSNIQSSASQPPRPQQGQVKLTMAQLTQLTQGHGGNQGLTVVIQGQGQTTGQLQLIPQGVTVLPGPGQQLMQAAMPNGTVQRFLFTPLATTATTASTTTTTVSTTAAGTGEQRQSKLSPQMQVHQDKTLPPAQSSSVGPAEAQPQTAQPSAQPQPQTQPQSPAQPEVQTQPEVQTQTTVSSHVPSEAQPTHAQSSKPQVAAQSQPQSNVQGQSPVRVQSPSQTRIRPSTPSQLSPGQQSQVQTTTSQPIPIQPHTSLQIPSQGQPQSQPQVQSSTQTLSSGQTLNQVTVSSPSRPQLQIQQPQPQVIAVPQLQQQVQVLSQIQSQVVAQIQAQQSGVPQQIKLQLPIQIQQSSAVQTHQIQNVVTVQAASVQEQLQRVQQLRDQQQKKKQQQIEIKREHTLQASNQSEIIQKQVVMKHNAVIEHLKQKKSMTPAEREENQRMIVCNQVMKYILDKIDKEEKQAAKKRKREESVEQKRSKQNATKLSALLFKHKEQLRAEILKKRALLDKDLQIEVQEELKRDLKIKKEKDLMQLAQATAVAAPCPPVTPAPPAPPAPPPSPPPPPAVQHTGLLSTPTLPAASQKRKREEEKDSSSKSKKKKMISTTSKETKKDTKLYCICKTPYDESKFYIGCDRCQNWYHGRCVGILQSEAELIDEYVCPQCQSTEDAMTVLTPLTEKDYEGLKRVLRSLQAHKMAWPFLEPVDPNDAPDYYGVIKEPMDLATMEERVQRRYYEKLTEFVADMTKIFDNCRYYNPSDSPFYQCAEVLESFFVQKLKGFKASRSHNNKLQSTAS

SEQ ID NO: 30 - protein, 2168 aa, Homo sapiens
MESGERLPSSAASSTTPTSSSTPSVASVVSKGGLSTGVASLSSTINPCGHLFRTAGDQPFNLSTVSSAFPMVSHPVFGLHSASSGHSEFGGLGTLGTPTALAAHPQLASFPGAEWWRTTDAHTRTGATFFPPLLGIPPLFAPPAQNHDSSSFHSRTSGKSNRNGPEKGVNGSINGSNTSSVIGINTSVLSTTASSSMGQTKSTSSGGGNRKCNQEQSKNQPLDARVDKIKDKKPRKKAMESSSNSDSDSGTSSDTSSEGISSSDSDDLEEDEEEEDQSIEESEDDDSDSESEAQHKSNNQVLLHGISDPKADGQKATEKAQEKRIHQPLPLASESQTHSFQSQQKQPQVLSQQLPFIFQSSQAKEESVNKHTSVIQSTGLVSNVKPLSLVNQAKKETYMKLIVPSPDVLKAGNKNTSEESSLLTSELRSKREQYKQAFPSQLKKQESSKSLKKVIAALSNPKATSSSPAHPKQTLENNHPNPFLTNALLGNHQPNGVIQSVIQEAPLALTTKTKMQSKINENIAAASSTPFSSPVNLSTSGRRTPGNQTPVMPSASPILHSQGKEKAVSNNVNPVKTQHHSHPAKSLVEQFRGTDSDIPSSKDSEDSNEDEEEDDEEEDEEDDEDDESDDSQSESDSNSESDTEGSEEEDDDDKDQDESDSDTEGEKTSMKLNKTTSSVKSPSMSLTGHSTPRNLHIAKAPGSAPAALCSESQSPAFLGTSSSTLTSSPHSGTSKRRRVTDERELRIPLEYGWQRETRIRNFGGRLQGEVAYYAPCGKKLRQYPEVIKYLSRNGIMDISRDNFSFSAKIRVGDFYEARDGPQGMQWCLLKEEDVIPRIRAMEGRRGRPPNPDRQRAREESRMRRRKGRPPNVGNAEFLDNADAKLLRKLQAQEIARQAAQIKLLRKLQKQEQARVAKEAKKQQAIMAAEEKRKQKEQIKIMKQQEKIKRIQQIRMEKELRAQQILEAKKKKKEEAANAKLLEAEKRIKEKEMRRQQAVLLKHQERERRRQHMMLMKAMEARKKAEEKERLKQEKRDEKRLNKERKLEQRRLELEMAKELKKPNEDMCLADQKPLPELPRIPGLVLSGSTFSDCLMVVQFLRNFGKVLGFDVNIDVPNLSVLQEGLLNIGDSMGEVQDLLVRLLSAAVCDPGLITGYKAKTALGEHLLNVGVNRDNVSEILQIFMEAHCGQTELTESLKTKAFQAHTPAQKASVLAFLINELACSKSVVSEIDKNIDYMSNLRRDKWVVEGKLRKLRIIHAKKTGKRDTSGGIDLGEEQHPLGTPTPGRKRRRKGGDSDYDDDDDDDSDDQGDEDDEDEEDKEDKKGKKTDICEDEDEGDQAASVEELEKQIEKLSKQQSQYRRKLFDASHSLRSVMFGQDRYRRRYWILPQCGGIFVEGMESGEGLEEIAKEREKLKKAESVQIKEEMFETSGDSLNCSNTDHCEQKEDLKEKDNTNLFLQKPGSFSKLSKLLEVAKMPPESEVMTPKPNAGANGCTLSYQNSGKHSLGSVQSTATQSNVEKADSNNLFNTGSSGPGKFYSPLPNDQLLKTLTEKNRQWFSLLPRTPCDDTSLTHADMSTASLVTPQSQPPSKSPSPTPAPLGSSAQNPVGLNPFALSPLQVKGGVSMMGLQFCGWPTGVVTSNIPFTSSVPSLGSGLGLSEGNGNSFLTSNVASSKSESPVPQNEKATSAQPAAVEVAKPVDFPSPKPIPEEMQFGWWRIIDPEDLKALLKVLHLRGIREKALQKQIQKHLDYITQACLKNKDVAIIELNENEENQVTRDIVENWSVEEQAMEMDLSVLQQVEDLERRVASASLQVKGWMCPEPASEREDLVYFEHKSFTKLCKEHDGEFTGEDESSAHALERKSDNPLDIAVTRLADLERNIERRIEEDIAPGLRVWRRALSEARSAAQVALCIQQLQKSIAWEKSIMKVYCQICRKGDNEELLLLCDGCDKGCHTYCHRPKITTIPDGDWFCPACIAKASGQTLKIKKLHVKGKKTNESKKGKKVTLTGDTEDEDSASTSSSLKRGNKDLKKRKMEENTSINLSKQESFTSVKKPKRDDSKDLALCSMILTEMETHEDAWPFLLPVNLKLVPGYKKVIKKPMDFSTIREKLSSGQYPNLETFALDVRLVFDNCETFNEDDSDIGRAGHNMRKYFEKKWTDTFKVS

SEQ ID NO: 31 - protein, 1390 aa, Homo sapiens
MVVLRSSLELHNHSAASATGSLDLSSDFLSLEHIGRRRLRSAGAAQKKPAATTAKAGDGSSVKEVETYHRTRALRSLRKDAQNSSDSSFEKNVEITEQLANGRHFTRQLARQQADKKKEEHREDKVIPVTRSLRARNIVQSTEHLHEDNGDVEVRRSCRIRSRYSGVNQSMLFDKLITNTAEAVLQKMDDMKKMRRQRMRELEDLGVFNETEESNLNMYTRGKQKDIQRTDEETTDNQEGSVESSEEGEDQEHEDDGEDEDDEDDDDDDDDDDDDDDEDDEDEEDGEEENQKRYYLRQRKATVYYQAPLEKPRHQRKPNIFYSGPASPARPRYRLSSAGPRSPYCKRMNRRRHAIHSSDSTSSSSSEDEQHFERRRKRSRNRAINRCLPLNFRKDELKGIYKDRMKIGASLADVDPMQLDSSVRFDSVGGLSNHIAALKEMVVFPLLYPEVFEKFKIQPPRGCLFYGPPGTGKTLVARALANECSQGDKRVAFFMRKGADCLSKWVGESERQLRLLFDQAYQMRPSIIFFDEIDGLAPVRSSRQDQIHSSIVSTLLALMDGLDSRGEIVVIGATNRLDSIDPALRRPGRFDREFLFSLPDKEARKEILKIHTRDWNPKPLDTFLEELAENCVGYCGADIKSICAEAALCALRRRYPQIYTTSEKLQLDLSSINISAKDFEVAMQKMIPASQRAVTSPGQALSTVVKPLLQNTVDKILEALQRVFPHAEFRTNKTLDSDISCPLLESDLAYSDDDVPSVYENGLSQKSSHKAKDNFNFLHLNRNACYQPMSFRPRILIVGEPGFGQGSHLAPAVIHALEKFTVYTLDIPVLFGVSTTSPEETCAQVIREAKRTAPSIVYVPHIHVWWEIVGPTLKATFTTLLQNIPSFAPVLLLATSDKPHSALPEEVQELFIRDYGEIFNVQLPDKEERTKFFEDLILKQAAKPPISKKKAVLQALEVLPVAPPPEPRSLTAEEVKRLEEQEEDTFRELRIFLRNVTHRLAIDKRFRVFTKPVDPDEVPDYVTVIKQPMDLSSVISKIDLHKYLTVKDYLRDIDLICSNALEYNPDRDPGDRLIRHRACALRDTAYAIIKEELDEDFEQLCEEIQESRKKRGCSSSKYAPSYYHVMPKQNSTLVGDKRSDPEQNEKLKTPSTPVACSTPAQLKRKIRKKSNWYLGTIKKRRKISQAKDDSQNAIDHKIESDTEETQDTSVDHNETGNTGESSVEENEKQQNASESKLELRNNSNTCNIENELEDSRKTTACTELRDKIACNGDASSSQIIHISDENEGKEMCVLRMTRARRSQVEQQQLITVEKALAILSQPTPSLVVDHERLKNLLKTVVKKSQNYNIFQLENLYAVISQCIYRHRKDHDKTSLIQKMEQEVENFSCSR

SEQ ID NO: 32 - protein, 1925 aa, Homo sapiens
MFTLTGCRLVEKTQKVENPSVSFASSFPLIPLLLRGKSVQKKQAESKSQIKLHTQSAPFGLCPKDMMLTQAPSSVVRSRNSRNHTVNSGGSCLSASTVAIPAINDSSAAMSACSTISAQPASSMDTQMHSPKKQERVNKRVIWGIEVAEELHWKGWELGKETTRNLVLKNRSLKLQKMKYRPPKTKFFFTVIPQPIFLSPGITLTLPIVFRPLEAKEYMDQLWFEKAEGMFCVGLRATLPCHRLICRPPSLQLPMCAVGDTTEAFFCLDNVGDLPTFFTWEFSSPFQMLPATGLLEPGQASQIKVTFQPLTAVIYEVQATCWYGAGSRQRSSIQLQAVAKCAQLLVSIKHKCPEDQDAEGFQKLLYFGSVAVGCTSERQIRLHNPSAVNAPFRIEISPDELAEDQAFSCPTAHGIVLPGEKKCVSVFFHPKTLDTRTVDYCSIMPSGCASKTLLKVVGFCRGPAVSLQHYCVNFSWVNLGERSEQPLWIENQSDCTAHFQFAIDCLESVFTIRPAFGTLVGKARMTLHCAFQPTHPIICFRRVACLIHHQDPLFLDLMGTCHSDSTKPAILKPQHLTWYRTHLARGLTLYPPDILDAMLKEKKLAQDQNGALMIPIQDLEDMPAPQYPYIPPMTEFFFDGTSDITIFPPPISVEPVEVDFGACPGPEAPNPVPLCLMNHTKGKIMVVWTRRSDCPFWVTPESCDVPPLKSMAMRLHFQPPHPNCLYTVELEAFAIYKVLQSYSNIEEDCTMCPSWCLTVRARGHSYFAGFEHHIPQYSLDVPKLFPAVSSGEPTYRSLLLVNKDCKLLTFSLAPQRGSDVILRPTSGLVAPGAHQIILICTYPEGSSWKQHTFYLQCNASPQYLKEVSMYSREEPLQLKLDTHKSLYFKPTWVGCSSTSPFTFRNPSRLPLQFEWRVSEQHRKLLAVQPSRGLIQPNERLTLTWTFSPLEETKYLFQVGMWVWEAGLSPNANPAATTHYMLRLVGVGLTSSLSAKEKELAFGNVLVNSKQSRFLVLLNDGNCTLYYRLYLEQGSPEAVDNHPLALQLDRTEGSMPPRSQDTICLTACPKQRSQYSWTITYSLLSHRDNKAGEKQELCCVSLVAVYPLLSILDVSSMGSAEGITRKHLWRLFSLDLLNSYLERDPTPCELTYKVPTRHSMSQIPPVLTPLRLDFNFGAAPFKAPPSVVFLALKNSGVVSLDWAFLLPSDQRIDVELWAEQAELNSTELHQMRVQDNCLFSISPKAGSLSPGQEQMVELKYSHLFIGTDHLPVLFKVSHGREILLNFIGVTVKPEQKYVHFTSTTHQFIPIPIGDTLPPRQIYELYNGGSVPVTYEVQTDVLSQVQEKNFDHPIFCCLNPKGEIQPGSTARVLWIFSPIEAKTYTVDVPIHILGWNSALIHFQGVGYNPHMMGDTAPFHNISSWDNSSIHSRLVVPGQNVFLSQSHISLGNIPVQSKCSRLLFLNNISKNEEIAFSWQPSPLDFGEVSVSPMIGVVAPEETVPFVVTLRASVHASFYSADLVCKLYSQQLMRQYHKELQEWKDEKVRQEVEFTITDMKVKKRTCCTACEPARKYKTLPPIKNQQSVSRPASWKLQTPKEEVSWPCPQPPSPGMLCLGLTARAHATDYFLANFFSEFPCHFLHRELPKRKAPREESETSEEKSPNKWGPVSKQKKQLLVDILTTIIRGLLEDKNFHEAVDQSLVEQVPYFRQFWNEQSTKFMDQKNSLYLMPILPVPSSSWEDGKGKQPKEDRPEHYPGLGKKEEGEEEKGEEEEEELEEEEEEEEETEEEELGKEEIEEKEEERDEKEEKVSWAGIGPTPQPESQESMQWQWQQQLNVMVKEEQEQDEKEAIRRLPAFANLQEALLENMIQNILVEASRGEVVLTSRPRVIALPPFCVPRSLTPDTLLPTQQAEVLHPVVPLPTDLP

SEQ ID NO: 33 - protein, 599 aa, Homo sapiens
MGPKRRQLTFREKSRIIQEVEENPDLRKGEIARRFNIPPSTLSTILKNKRAILASERKYGVASTCRKTNKLSPYDKLEGLLIAWFQQIRAAGLPVKGIILKEKALRIAEELGMDDFTASNGWLDRFRRRHGVVSCSGVARARARNAAPRTPAAPASPAAVPSEGSGGSTTGWRAREEQPPSVAEGYASQDVFSATETSLWYDFLPDQAAGLCGGDGRPRQATQRLSVLLCANADGSEKLPPLVAGKSAKPRAGQAGLPCDYTANSKGGVTTQALAKYLKALDTRMAAESRRVLLLAGRLAAQSLDTSGLRHVQLAFFPPGTVHPLERGVVQQVKGHYRQAMLLKAMAALEGQDPSGLQLGLTEALHFVAAAWQAVEPSDIAACFREAGFGGGPNATITTSLKSEGEEEEEEEEEEEEEEGEGEEEEEEGEEEEEEGGEGEELGEEEEVEEEGDVDSDEEEEEDEESSSEGLEAEDWAQGVVEAGGSFGAYGAQEEAQCPTLHFLEGGEDSDSDSEEEDDEEEDDEDEDDDDDEEDGDEVPVPSFGEAMAYFAMVKRYLTSFPIDDRVQSHILHLEHDLVHVTRKNHARQAGVRGLGHQS

SEQ ID NO: 34 - protein, 1759 aa, Homo sapiens
MDLGTAEGTRCTDPPAGKPAMAPKRKGGLKLNAICAKLSRQVVVEKRADAGSHTEGSPSQPRDQERSGPESGAARAPRSEEDKRRAVIEKWVNGEYSEEPAPTPVLGRIAREGLELPPEGVYMVQPQGCSDEEDHAEEPSKDGGALEEKDSDGAASKEDSGPSTRQASGEASSLRDYAASTMTEFLGMFGYDDQNTRDELARKISFEKLHAGSTPEAATSSMLPTSEDTLSKRARFSKYEEYIRKLKAGEQLSWPAPSTKTEERVGKEVVGTLPGLRLPSSTAHLETKATILPLPSHSSVQMQNLVARASKYDFFIQKLKTGENLRPQNGSTYKKPSKYDLENVKYLHLFKPGEGSPDMGGAIAFKTGKVGRPSKYDVRGIQKPGPAKVPPTPSLAPAPLASVPSAPSAPGPGPEPPASLSFNTPEYLKSTFSKTDSITTGTVSTVKNGLPTDKPAVTEDVNIYQKYIARFSGSQHCGHIHCAYQYREHYHCLDPECNYQRFTSKQDVIRHYNMHKKRDNSLQHGFMRFSPLDDCSVYYHGCHLNGKSTHYHCMQVGCNKVYTSTSDVMTHENFHKKNTQLINDGFQRFRATEDCGTADCQFYGQKTTHFHCRRPGCTFTFKNKCDIEKHKSYHIKDDAYAKDGFKKFYKYEECKYEGCVYSKATNHFHCIRAGCGFTFTSTSQMTSHKRKHERRHIRSSGALGLPPSLLGAKDTEHEESSNDDLVDFSALSSKNSSLSASPTSQQSSASLAAATAATEAGPSATKPPNSKISGLLPQGLPGSIPLALALSNSGLPTPTPYFPILAGRGSTSLPVGTPSLLGAVSSGSAASATPDTPTLVASGAGDSAPVAAASVPAPPASIMERISASKGLISPMMARLAAAALKPSATFDPGSGQQVTPARFPPAQVKPEPGESTGAPGPHEASQDRSLDLTVKEPSNESNGHAVPANSSLLSSLMNKMSQGNPGLGSLLNIKAEAEGSPAAEPSPFLGKAVKALVQEKLAEPWKVYLRRFGTKDFCDGQCDFLHKAHFHCVVEECGALFSTLDGAIKHANFHFRTEGGAAKGNTEAAFPASAAETKPPMAPSSPPVPPVTTATVSSLEGPAPSPASVPSTPTLLAWKQLASTIPQMPQIPASVPHLPASPLATTSLENAKPQVKPGFLQFQENDPCLATDCKYANKFHFHCLFGNCKYVCKTSGKAESHCLDHINPNNNLVNVRDQFAYYSLQCLCPNQHCEFRMRGHYHCLRTGCYFVTNITTKLPWHIKKHEKAERRAANGFKYFTKREECGRLGCKYNQVNSHFHCIREGCQFSFLLKHQMTSHARKHMRRMLGKNFDRVPPSQGPPGLMDAETDECMDYTGCSPGAMSSESSTMDRSCSSTPVGNESTAAGNTISMPTASGAKKRFWIIEDMSPFGKRRKTASSRKMLDEGMMLEGFRRFDLYEDCKDAACQFSLKVTHYHCTRENCGYKFCGRTHMYKHAQHHDRVDNLVLDDFKRFKASLSCHFADCPFSGTSTHFHCLRCRFRCTDSTKVTAHRKHHGKQDVISAAGFCQFSSSADCAVPDCKYKLKCSHFHCTFPGCRHTVVGMSQMDSHKRKHEKQERGEPAAEGPAPGPPISLDGSLSLGAEPGSLLFLQSAAAGLGLALGDAGDPGPPDAAAPGPREGAAAAAAAAGESSQEDEEEELELPEEEAEDDEDEDDDEDDDDEDDDEDDDDEDLRTDSEESLPEAAAEAAGAGARTPALAALAALGAPGPAPTAASSP

SEQ ID NO: 35 - protein, 406 aa, Homo sapiens
MTQTLDTREDPLNLGGGGGGGCGCGWAHSASLSSWSSCHRRRPGAPAYNRPHRYSPKTEYGPPRKQPKQQHGPGFWFQPPVCSNWGCWGGPWRPPPPGFWKFPCPVQVFRVYGLHPLCFCCCSCWSGSWNPGWVKPPGRKKRWGRRGRGLRHHPRHSYPRSPPADVSTLPRPVKLYEWREPGMRAPPNTTQFIMNQIYEDMRQQEKVERQQEALRAQKATVSGEASPARSSGNDAPPGGSKETWGLQETLYGFVQNPSLAFSPNPEENQSLAPLLVEEEEEKKNDDEEEYDQEVCDAKEASEEEEEVEDEEEEVEDEEEEEVEEAEYVEEGEEELEEEELEEEEEVLEENEQRGEEFHLPLEMPLSIFVEAEEKRENFISCTFLNPEQIIPKVPQESLFMAQDFNC

SEQ ID NO: 36 - protein, 631 aa, Homo sapiens
MSHQEGSTDGLPDLGTESLFSSPEEQSGAVAATEASSDIDIATSELSVTVTGDGSDSRDGGFPNDASTENRSSDQESASEDIELESLEDFEHFLMSGESLFHYPLVGEEETEREEEDEEIQEEGGEEEEEEEEEEEEEEEEEEEEEEQPRAGPQGSGGNHEQYSLEEDQALEEWVSSETSALPRPRWQVVTALHQRQLGSRPRFVYEACGARAFVQRFRLQYRLADHVGCVNTVHFNQRGTRLASSGDDLKVIVWDWVRQRPVLNFESGHTNNVFQAKFLPNCGDSTLAMCARDGQVRVAELINASYFNNTKCVAQHRGPAHKLALEPDSPYKFLTSGEDAVVFTIDLRQDRPASKVVVTRENDKKVGLYTITVNPANTYQFAVGGQDQFVRIYDQRKIDKKENNGVLKKFTPHHLVNCDFPTNITCVVYSHDGTELLASYNDDDIYLFNSSHSDGAQYSKRFKGHRNNTTVKGVNFYGPRSEFVVSGSDCGHIFFWEKSSCQIIQFLKGSREGTINCLEPHPYLPVLACSGLDHDVKIWTPTAKAATELTGLKKVIKKNKWERDEDSLHHGSLFDQYMLWFLLRHVTQRGRHQDWRSGEAEFPDEESDESSSTSETSEEEVQDRVQCMPS

SEQ ID NO: 37 - protein, 740 aa, Homo sapiens
MATANSIIVLDDDDEDEAAAQPGPSHPLPNAASPGAEAPSSSEPHGARGSSSSGGKKCYKLENEKLFEEFLELCKMQTADHPEVVPFLYNRQQRAHSLFLASAEFCNILSRVLSRARSRPAKLYVYINELCTVLKAHSAKKKLNLAPAATTSNEPSGNNPPTHLSLDPTNAENTASQSPRTRGSRRQIQRLEQLLALYVAEIRRLQEKELDLSELDDPDSAYLQEARLKRKLIRLFGRLCELKDCSSLTGRVIEQRIPYRGTRYPEVNRRIERLINKPGPDTFPDYGDVLRAVEKAAARHSLGLPRQQLQLMAQDAFRDVGIRLQERRHLDLIYNFGCHLTDDYRPGVDPALSDPVLARRLRENRSLAMSRLDEVISKYAMLQDKSEEGERKKRRARLQGTSSHSADTPEASLDSGEGPSGMASQGCPSASRAETDDEDDEESDEEEEEEEEEEEEEATDSEEEEDLEQMQEGQEDDEEEDEEEEAAAGKDGDKSPMSSLQISNEKNLEPGKQISRSSGEQQNKGRIVSPSLLSEEPLAPSSIDAESNGEQPEELTLEEESPVSQLFELEIEALPLDTPSSVETDISSSRKQSEEPFTTVLENGAGMVSSTSFNGGVSPHNWGDSGPPCKKSRKEKKQTGSGPLGNSYVERQRSVHEKNGKKICTLPSPPSPLASLAPVADSSTRVDSPSHGLVTSSLCIPSPARLSQTPHSQPPRPGTCKTSVATQCDPEEIIVLSDSD

SEQ ID NO: 38 - protein, 1507 aa, Homo sapiens
MTTVVVHVDSKAELTTLLEQWEKEHGSGQDMVPILTRMSQLIEKETEEYRKGDPDPFDDRHPGRADPECMLGHLLRILFKNDDFMNALVNAYVMTSREPPLNTAACRLLLDIMPGLETAVVFQEKEGIVENLFKWAREADQPLRTYSTGLLGGAMENQDIAANYRDENSQLVAIVLRRLRELQLQEVALRQENKRPSPRKLSSEPLLPLDEEAVDMDYGDMAVDVVDGDQEEASGDMEISFHLDSGHKTSSRVNSTTKPEDGGLKKNKSAKQGDRENFRKAKQKLGFSSSDPDRMFVELSNSSWSEMSPWVIGTNYTLYPMTPAIEQRLILQYLTPLGEYQELLPIFMQLGSRELMMFYIDLKQTNDVLLTFEALKHLASLLLHNKFATEFVAHGGVQKLLEIPRPSMAATGVSMCLYYLSYNQDAMERVCMHPHNVLSDVVNYTLWLMECSHASGCCHATMFFSICFSFRAVLELFDRYDGLRRLVNLISTLEILNLEDQGALLSDDEIFASRQTGKHTCMALRKYFEAHLAIKLEQVKQSLQRTEGGILVHPQPPYKACSYTHEQIVEMMEFLIEYGPAQLYWEPAEVFLKLSCVQLLLQLISIACNWKTYYARNDTVRFALDVLAILTVVPKIQLQLAESVDVLDEAGSTVSTVGISIILGVAEGEFFIHDAEIQKSALQIIINCVCGPDNRISSIGKFISGTPRRKLPQNPKSSEHTLAKMWNVVQSNNGIKVLLSLLSIKMPITDADQIRALACKALVGLSRSSTVRQIISKLPLFSSCQIQQLMKEPVLQDKRSDHVKFCKYAAELIERVSGKPLLIGTDVSLARLQKADVVAQSRISFPEKELLLLIRNHLISKGLGETATVLTKEADLPMTAASHSSAFTPVTAAASPVSLPRTPRIANGIATRLGSHAAVGASAPSAPTAHPQPRPPQGPLALPGPSYAGNSPLIGRISFIRERPSPCNGRKIRVLRQKSDHGAYSQSPAIKKQLDRHLPSPPTLDSIITEYLREQHARCKNPVATCPPFSLFTPHQCPEPKQRRQAPINFTSRLNRRASFPKYGGVDGGCFDRHLIFSRFRPISVFREANEDESGFTCCAFSARERFLMLGTCTGQLKLYNVFSGQEEASYNCHNSAITHLEPSRDGSLLLTSATWSQPLSALWGMKSVFDMKHSFTEDHYVEFSKHSQDRVIGTKGDIAHIYDIQTGNKLLTLFNPDLANNYKRNCATFNPTDDLVLNDGVLWDVRSAQAIHKFDKFNMNISGVFHPNGLEVIINTEIWDLRTFHLLHTVPALDQCRVVFNHTGTVMYGAMLQADDEDDLMEERMKSPFGSSFRTFNATDYKPIATIDVKRNIFDLCTDTKDCYLAVIENQGSMDALNMDTVCRLYEVGRQRLAEDEDEEEDQEEEEQEEEDDDEDDDDTDDLDELDTDQLLEAELEEDDNNENAGEDGDNDFSPSDEELANLLEEGEDGEDEDSDADEEVELILGDTDSSDNSDLEDDIILSLNE

SEQ ID NO: 39 - protein, 251 aa, Homo sapiens
MDMKRRIHLELRNRTPAAVRELVLDNCKSNDGKIEGLTAEFVNLEFLSLINVGLISVSNLPKLPKLKKLELSENRIFGGLDMLAEKLPNLTHLNLSGNKLKDISTLEPLKKLECLKSLDLFNCEVTNLNDYRESVFKLLPQLTYLDGYDREDQEAPDSDAEVDGVDEEEEDEEGEDEEDEDDEDGEEEEFDEEDDEDEDVEGDEDDDEVSEEEEEFGLDEEDEDEDEDEEEEEGGKGEKRKRETDDEGEDD

SEQ ID NO: 40 - protein, 1312 aa, Homo sapiens
MKALDEPPYLTVGTDVSAKYRGAFCEAKIKTAKRLVKVKVTFRHDSSTVEVQDDHIKGPLKVGAIVEVKNLDGAYQEAVINKLTDASWYTVVFDDGDEKTLRRSSLCLKGERHFAESETLDQLPLTNPEHFGTPVIGKKTNRGRRSNHIPEEESSSSSSDEDEDDRKQIDELLGKVVCVDYISLDKKKALWFPALVVCPDCSDEIAVKKDNILVRSFKDGKFTSVPRKDVHEITSDTAPKPDAVLKQAFEQALEFHKSRTIPANWKTELKEDSSSSEAEEEEEEEDDEKEKEDNSSEEEEEIEPFPEERENFLQQLYKFMEDRGTPINKRPVLGYRNLNLFKLFRLVHKLGGFDNIESGAVWKQVYQDLGIPVLNSAAGYNVKCAYKKYLYGFEEYCRSANIEFQMALPEKVVNKQCKECENVKEIKVKEENETEIKEIKMEEERNIIPREEKPIEDEIERKENIKPSLGSKKNLLESIPTHSDQEKEVNIKKPEDNENLDDKDDDTTRVDESLNIKVEAEEEKAKSGDETNKEEDEDDEEAEEEEEEEEEEEDEDDDDNNEEEEFECYPPGMKVQVRYGRGKNQKMYEASIKDSDVEGGEVLYLVHYCGWNVRYDEWIKADKIVRPADKNVPKIKHRKKIKNKLDKEKDKDEKYSPKNCKLRRLSKPPFQTNPSPEMVSKLDLTDAKNSDTAHIKSIEITSILNGLQASESSAEDSEQEDERGAQDMDNNGKEESKIDHLTNNRNDLISKEEQNSSSLLEENKVHADLVISKPVSKSPERLRKDIEVLSEDTDYEEDEVTKKRKDVKKDTTDKSSKPQIKRGKRRYCNTEECLKTGSPGKKEEKAKNKESLCMENSSNSSSDEDEEETKAKMTPTKKYNGLEEKRKSLRTTGFYSGFSEVAEKRIKLLNNSDERLQNSRAKDRKDVWSSIQGQWPKKTLKELFSDSDTEAAASPPHPAPEEGVAEESLQTVAEEESCSPSVELEKPPPVNVDSKPIEEKTVEVNDRKAEFPSSGSNSVLNTPPTTPESPSSVTVTEGSRQQSSVTVSEPLAPNQEEVRSIKSETDSTIEVDSVAGELQDLQSEGNSSPAGFDASVSSSSSNQPEPEHPEKACTGQKRVKDAQGGGSSSKKQKRSHKATVVNNKKKGKGTNSSDSEELSAGESITKSQPVKSVSTGMKSHSTKSPARTQSPGKCGKNGDKDPDLKEPSNRLPKVYKWSFQMSDLENMTSAERITILQEKLQEIRKHYLSLKSEVASIDRRRKRLKKKERESAATSSSSSSPSSSSITAAVMLTLAEPSMSSASQNGMSVECR

SEQ ID NO: 41 - protein, 249 aa, Homo sapiens
MEMGRRIHLELRNRTPSDVKELVLDNSRSNEGKLEGLTDEFEELEFLSTINVGLTSIANLPKLNKLKKLELSDNRVSGGLEVLAEKCPNLTHLNLSGNKIKDLSTIEPLKKLENLKSLDLFNCEVTNLNDYRENVFKLLPQLTYLDGYDRDDKEAPDSDAEGYVEGLDDEEEDEDEEEYDEDAQVVEDEEDEDEEEEGEEEDVSGEEEEDEEGYNDGEVDDEEDEEELGEEERGQKRKREPEDEGEDDD

SEQ ID NO: 42 - protein, 268 aa, Homo sapiens
MEMKKKINLELRNRSPEEVTELVLDNCLCVNGEIEGLNDTFKELEFLSMANVELSSLARLPSLNKLRKLELSDNIISGGLEVLAEKCPNLTYLNLSGNKIKDLSTVEALQNLKNLKSLDLFNCEITNLEDYRESIFELLQQITYLDGFDQEDNEAPDSEEEDDEDGDEDDEEEEENEAGPPEGYEEEEEEEEEEDEDEDEDEDEAGSELGEGEEEVGLSYLMKEEIQDEEDDDDYVEEGEEEEEEEEGGLRGEKRKRDAEDDGEEEDD

SEQ ID NO: 43 - protein, 764 aa, Homo sapiens
MNGEADCPTDLEMAAPKGQDRWSQEDMLTLLECMKNNLPSNDSSKFKTTESHMDWEKVAFKDFSGDMCKLKWVEISNEVRKFRTLTELILDAQEHVKNPYKGKKLKKHPDFPKKPLTPYFRFFMEKRAKYAKLHPEMSNLDLTKILSKKYKELPEKKKMKYIQDFQREKQEFERNLARFREDHPDLIQNAKKSDIPEKPKTPQQLWYTHEKKVYLKVRPDATTKEVKDSLGKQWSQLSDKKRLKWIHKALEQRKEYEEIMRDYIQKHPELNISEEGITKSTLTKAERQLKDKFDGRPTKPPPNSYSLYCAELMANMKDVPSTERMVLCSQQWKLLSQKEKDAYHKKCDQKKKDYEVELLRFLESLPEEEQQRVLGEEKMLNINKKQATSPASKKPAQEGGKGGSEKPKRPVSAMFIFSEEKRRQLQEERPELSESELTRLLARMWNDLSEKKKAKYKAREAALKAQSERKPGGEREERGKLPESPKRAEEIWQQSVIGDYLARFKNDRVKALKAMEMTWNNMEKKEKLMWIKKAAEDQKRYERELSEMRAPPAATNSSKKMKFQGEPKKPPMNGYQKFSQELLSNGELNHLPLKERMVEIGSRWQRISQSQKEHYKKLAEEQQKQYKVHLDLWVKSLSPQDRAAYKEYISNKRKSMTKLRGPNPKSSRTTLQSKSESEEDDEEDEDDEDEDEEEEDDENGDSSEDGGDSSESSSEDESEDGDENEEDDEDEDDDEDDDEDEDNESEGSSSSSSSSGDSSDSDSN

SEQ ID NO: 44 - protein, 1812 aa, Homo sapiens
MAVDSAMELLFLDTFKHPSAEQSSHIDVVRFPCVVYINEVRVIPPGVRAHSSLPDNRAYGETSPHTFQLDLFFNNVSKPSAPVFDRLGSLEYDENTSIIFRPNSKVNTDGLVLRGWYNCLTLAIYGSVDRVISHDRDSPPPPPPPPPPPQPQPSLKRNPKHADGEKEDQFNGSPPRPQPRGPRTPPGPPPPDDDEDDPVPLPVSGDKEEDAPHREDYFEPISPDRNSVPQEGQYSDEGEVEEEQQEEGEEDEDDVDVEEEEDEDEDDRRTVDSIPEEEEEDEEEEGEEDEEGEGDDGYEQISSDEDGIADLERETFKYPNFDVEYTAEDLASVPPMTYDPYDRELVPLLYFSCPYKTTFEIEISRMKDQGPDKENSGAIEASVKLTELLDLYREDRGAKWVTALEEIPSLIIKGLSYLQLKNTKQDSLGQLVDWTMQALNLQVALRQPIALNVRQLKAGTKLVSSLAECGAQGVTGLLQAGVISGLFELLFADHVSSSLKLNAFKALDSVISMTEGMEAFLRGRQNEKSGYQKLLELILLDQTVRVVTAGSAILQKCHFYEVLSEIKRLGDHLAEKTSSLPNHSEPDHDTDAGLERTNPEYENEVEASMDMDLLESSNISEGEIERLINLLEEVFHLMETAPHTMIQQPVKSFPTMARITGPPERDDPYPVLFRYLHSHHFLELVTLLLSIPVTSAHPGVLQATKDVLKFLAQSQKGLLFFMSEYEATNLLIRALCHFYDQDEEEGLQSDGVIDDAFALWLQDSTQTLQCITELFSHFQRCTASEETDHSDLLGTLHNLYLITFNPVGRSAVGHVFSLEKNLQSLITLMEYYSKEALGDSKSKKSVAYNYACILILVVVQSSSDVQMLEQHAASLLKLCKADENNAKLQELGKWLEPLKNLRFEINCIPNLIEYVKQNIDNLMTPEGVGLTTALRVLCNVACPPPPVEGQQKDLKWNLAVIQLFSAEGMDTFIRVLQKLNSILTQPWRLHVNMGTTLHRVTTISMARCTLTLLKTMLTELLRGGSFEFKDMRVPSALVTLHMLLCSIPLSGRLDSDEQKIQNDIIDILLTFTQGVNEKLTISEETLANNTWSLMLKEVLSSILKVPEGFFSGLILLSELLPLPLPMQTTQVIEPHDISVALNTRKLWSMHLHVQAKLLQEIVRSFSGTTCQPIQHMLRRICVQLCDLASPTALLIMRTVLDLIVEDLQSTSEDKEKQYTSQTTRLLALLDALASHKACKLAILHLINGTIKGDERYAEIFQDLLALVRSPGDSVIRQQCVEYVTSILQSLCDQDIALILPSSSEGSISELEQLSNSLPNKELMTSICDCLLATLANSESSYNCLLTCVRTMMFLAEHDYGLFHLKSSLRKNSSALHSLLKRVVSTFSKDTGELASSFLEFMRQILNSDTIGCCGDDNGLMEVEGAHTSRTMSINAAELKQLLQSKEESPENLFLELEKLVLEHSKDDDNLDSLLDSVVGLKQMLESSGDPLPLSDQDVEPVLSAPESLQNLFNNRTAYVLADVMDDQLKSMWFTPFQAEEIDTDLDLVKVDLIELSEKCCSDFDLHSELERSFLSEPSSPGRTKTTKGFKLGKHKHETFITSSGKSEYIEPAKRAHVVPPPRGRGRGGFGQGIRPHDIFRQRKQNTSRPPSMHVDDFVAAESKEVVPQDGIPPPKRPLKVSQKISSRGGFSGNRGGRGAFHSQNRFFTPPASKGNYSRREGTRGSSWSAQNTPRGNYNESRGGQSNFNRGPLPPLRPLSSTGYRPSPRDRASRGRGGLGPSWASANSGSGGSRGKFVSGGSGRGRHVRSFTR

SEQ ID NO: 45 - protein, 1923 aa, Homo sapiens [sequence omitted]

SEQ ID NO: 46 - protein, 19 aa, synthetic construct
SKLSYEQDGLHAGSPAALE

SEQ ID NO: 47 - DNA, 21 bp, synthetic construct
  source 1..21: mol_type = other DNA
gcctgataccttccctgacta

SEQ ID NO: 48 - DNA, 21 bp, synthetic construct
  source 1..21: mol_type = other DNA
gccacacaatgcgatccagaa

SEQ ID NO: 49 - protein, 50 aa, Homo sapiens
EELELPEEEAEDDEDEDDDEDDDDEDDDEDDDDEDLRTDSEESLPEAAAE

SEQ ID NO: 50 - protein, 50 aa, Homo sapiens
EEDEDSEKEEEEEDKEMEELQEEKECEKPQGDEEEEEEEEEVEEEEVEEA

SEQ ID NO: 51 - protein, 50 aa, Homo sapiens
EEEEEEEEEEGGGSGREEEEEEEGGSQGEEEEEEEDGHSEQEEEEEEEEE

SEQ ID NO: 52 - protein, 50 aa, Homo sapiens
DEEEEEEEEEEEEEEEEEEEEEEEEEEEEEEEEAAPDVIFQEDTSHTSAQ

SEQ ID NO: 53 - protein, 50 aa, Homo sapiens
EEEEIEEEDEDDDEDGEDVEDEEEEEEEEEEEEEEEENEDHQMNCHNTRI

SEQ ID NO: 54 - protein, 50 aa, Homo sapiens
STSDDKRASLDEDEDDDDEEDNDEDDNDEDDDDEDDDEAEDNDEDEDDDE

SEQ ID NO: 55 - protein, 50 aa, Homo sapiens
AEEEKAKSGDETNKEEDEDDEEAEEEEEEEEEEEDEDDDDNNEEEEFECY

SEQ ID NO: 56 - protein, 50 aa, Homo sapiens
IEVKENPEEEEEEEEEEEEDEESEEEEEEEGESEGSEGDEEDEKVSDEKD

SEQ ID NO: 57 - protein, 50 aa, Homo sapiens
SDGGDSEEEEEEEEEQEEEEEEEEQEEEEEEEEEEEEKGNEEPLSLDWPE

SEQ ID NO: 58 - protein, 50 aa, Homo sapiens
EEDMVSEEEEEEDGDAEETQDSEDDEEDEMEEDDDDSDYPEEMEDDDDDA

SEQ ID NO: 59 - protein, 50 aa, Homo sapiens
EEEAEEEEEEEEAEEEEEEEEEEEEEEEDEGCKGLFPSELDEELEDRPHE

SEQ ID NO: 60 - protein, 50 aa, Homo sapiens
EEEDDEDEDDDDDEDDEDDDDEDDEEEEEEEEEEPVKEAPGKRKKEMAKQ

SEQ ID NO: 61 - protein, 50 aa, Homo sapiens
DEEVDEDGEEEEEEEEEEEEEEEEEEEEYEQDERDQKEEGNDYDTRSEAS

SEQ ID NO: 62 - protein, 50 aa, Homo sapiens
EEGGEDDDDDDDDGDEGEEELEDIDEGDEDEGEEDEDDDEGEEGEEDEGE

SEQ ID NO: 63 - protein, 50 aa, Homo sapiens
DDEEGEGEEDDDDDEEEEGLEDIDEEGDEDEGEEDEDDDEGEEGEEDEGE

SEQ ID NO: 64 - protein, 50 aa, Homo sapiens
QDEEQRLKEEEEDKKRKEEEEAEDKEDDEDKDEDEEDEEDKEEDEEEDVP

SEQ ID NO: 65 - protein, 50 aa, Homo sapiens
EEEDEEEEGEEEDEEEKEEDEEETAQEKEDEEKEEEEAAEGEKEEGLEEG

SEQ ID NO: 66 - protein, 50 aa, Homo sapiens
GESLFHYPLVGEEETEREEEDEEIQEEGGEEEEEEEEEEEEEEEEEEEEE

SEQ ID NO: 67 - protein, 50 aa, Homo sapiens
EDEEEEEEGEEEEEEAEEEEEEDEEEEEEEEEEEEEEPQQRGQGEKSATP

SEQ ID NO: 68 - protein, 50 aa, Homo sapiens
QEHEDDGEDEDDEDDDDDDDDDDDDDDEDDEDEEDGEEENQKRYYLRQRK

SEQ ID NO: 69 - protein, 50 aa, Homo sapiens
EEEEDDDEDSGAEIEDDDEEGFDDEDEFDDEHDDDLDTEDNELEELEERA

SEQ ID NO: 70 - protein, 50 aa, Homo sapiens
DEDDSQDEEEEEEEDEEDDQEDDEGEEGDEDDDDDGSEMELDEDYPDMNA

SEQ ID NO: 71 - protein, 50 aa, Homo sapiens
SEEEEEEEDEEEEEEEEEEEEDEEEEEEEEEEEEEENIQSSPPRLTKPQS

SEQ ID NO: 72 - protein, 50 aa, Homo sapiens
SDEAEEEEEDSEECSEEEDGYSSEEAENEEDEDDTEEAEEDDEEEEEEMM

SEQ ID NO: 73 - protein, 50 aa, Homo sapiens
EEEEDEEEEEEDEEEEEEEEEEEEEEEEEEEEEEEAAAAVALGEVLGPRS

SEQ ID NO: 74 - protein, 50 aa, Homo sapiens
EDDEDEDVEGDEDDDEVSEEEEEFGLDEEDEDEDEDEEEEEGGKGEKRKR

SEQ ID NO: 75 - protein, 50 aa, Homo sapiens
VEDEEDEDEEEEGEEEDVSGEEEEDEEGYNDGEVDDEEDEEELGEEERGQ

SEQ ID NO: 76 - protein, 50 aa, Homo sapiens
DEDDEEEEENEAGPPEGYEEEEEEEEEEDEDEDEDEDEAGSELGEGEEEV

SEQ ID NO: 77 - protein, 50 aa, Homo sapiens
EEDDENGDSSEDGGDSSESSSEDESEDGDENEEDDEDEDDDEDDDEDEDN

SEQ ID NO: 78 - protein, 50 aa, Homo sapiens
EDEDDVDVEEEEDEDEDDRRTVDSIPEEEEEDEEEEGEEDEEGEGDDGYE

SEQ ID NO: 79 - protein, 50 aa, Homo sapiens
SDEEEEEEEEEEEEEATDSEEEEDLEQMQEGQEDDEEEDEEEEAAAGKDG

SEQ ID NO: 80 - protein, 50 aa, Homo sapiens
EDEDEEEDQEEEEQEEEDDDEDDDDTDDLDELDTDQLLEAELEEDDNNEN

SEQ ID NO: 81 - protein, 50 aa, Homo sapiens
VEDEEEEVEDEEEEEVEEAEYVEEGEEELEEEELEEEEEVLEENEQRGEE

SEQ ID NO: 82 - protein, 49 aa, Homo sapiens
KEEGEEEKGEEEEEELEEEEEEEETEEEELGKEEIEEKEEERDEKEEKV

SEQ ID NO: 83 - protein, 50 aa, Homo sapiens
GEEEEEEGEEEEEEGCEGEELGEEEEVEEEGDVDSDEEEEEDEESSSEGL

SEQ ID NO: 84 - protein, 50 aa, Homo sapiens
DEEEDDEEEDEEDDEDDESDDSQSESDSNSESDTEGSEEEDDDDKDQDES

SEQ ID NO: 85 - protein, 50 aa, Homo sapiens
EEEEEEEEEMTDESEEDGEEKVEKEEKEEELEEEEEKEEEEEEEGNQETA

SEQ ID NO: 86 - protein, 50 aa, Homo sapiens
EEEEEEFEEEFEEEEGELEEEEEEEDEEEEEELEEVEDLEFGTAGGEVEE

SEQ ID NO: 87 - protein, 50 aa, Homo sapiens
VEEEENGAEEEEEETAEDGEEEDEGEEEDEEEEEEDDEGPALKRAAEEED

SEQ ID NO: 88 - protein, 50 aa, Homo sapiens
GDEEEEGEEEEEEEEAGYGDGYDDWETDEDEESLGDEEEEEEEEEEEVLE

SEQ ID NO: 89 - protein, 50 aa, Homo sapiens
DDKEDDDDDEDDDDEEDEEEEEEEEEDDDDDTEDFADQENLPDPQDISCH

SEQ ID NO: 90 - protein, 50 aa, Homo sapiens
EEEEEEEEVEEEEEEVEEEEEEVEEEEEEVVEEELVGEEQELEAPETFSE

SEQ ID NO: 91 - protein, 50 aa, Homo sapiens
LDQLESEEGQEDEDEEDEEDEDEEEEDEEEDEEDKDADSLDEGLDGDTEL

SEQ ID NO: 92 - protein, 50 aa, Homo sapiens
KEEEEEKEEEEEEGEEEGGGEEGEEGGGGGEGEETEEEEEEEEEEEEEEQ

SEQ ID NO: 93 - protein, 50 aa, Homo sapiens
EEEEEAEEEEEEEDDDDDDSDDRDESENDDEDTALSEASEKDEGDSDEEE

(19) United States
(12) Patent Application Publication
(10) Pub. No.: US 2024/0342241 A1
(43) Pub. Date: Oct. 17, 2024

(54) Compositions and Methods for Treatment and Prevention of Misfolded Proteins

(71) Applicant: The Trustees of the University of Pennsylvania, Philadelphia, PA (US)

(72) Inventors: Xiaolu Yang, Wynnewood, PA (US); Liangqian Huang, Philadelphia, PA (US)

(21) Appl. No.: 18/681,395

(22) PCT Filed: Aug. 5, 2022
(86) PCT No.: PCT/US22/74614

§ 371 Date: Feb. 5, 2024

Related U.S. Application Data
(60) Provisional application No. 63/230,443, filed on Aug. 6, 2021.

(51) Int. Cl. A61K 38/17; A61K 38/16; A61K 48/00; C07K 16/18; C12N 15/113

(52) U.S. Cl. CPC A61K 38/1709; A61K 38/162; A61K 48/0033; C07K 16/18; C12N 15/113; C07K 2317/75; C12N 2310/11; C12N 2310/12

(57) ABSTRACT

The present invention relates to compositions and methods for promoting the removal of misfolded proteins and protein aggregates. The compositions and methods may be used to treat or prevent a disorder associated with misfolded proteins or protein aggregates. In certain instances, the compositions and methods relate to modulators of one or more poly-D/E protein.

### CROSS REFERENCE TO RELATED APPLICATION

This application claims priority to and the benefit of U.S. provisional application No. 63/230,443, filed Aug. 6, 2021, the content of which is incorporated herein in its entirety.

### STATEMENT REGARDING FEDERALLY SPONSORED RESEARCH OR DEVELOPMENT

This invention was made with government support under CA182675 and CA184867 awarded by the National Institutes of Health. The government has certain rights in the invention.

### BACKGROUND OF THE INVENTION

Protein quality control (PQC) systems are critical for cellular function and organismal health. At present, known PQC systems are mostly multicomponent machineries that operate via ATP-regulated interactions with non-native proteins to prevent aggregation and promote folding (Balchin, D., et al., Science 353, aac4354, 2016), and few systems that can broadly enable protein folding by a different mechanism have been identified. Moreover, proteins that contain the extensively charged poly-Asp/Glu (polyD/E) region are common in eukaryotic proteomes (Karlin, S., et al., Proc Natl Acad Sci USA 99, 333-338, 2002), but their biochemical activities remain undefined.

Thus, there is a need in the art for compositions and methods for ATP-independent elimination of misfolded proteins. The present invention satisfies this unmet need.

### SUMMARY OF THE INVENTION

In one embodiment, the present invention relates to a composition for treating or preventing a disease or disorder associated with misfolded protein or protein aggregates, the composition comprising a modulator of one or more poly-D/E protein. In one embodiment, the modulator increases the expression or activity of the one or more poly-D/E protein. In one embodiment, the modulator is at least one of the group consisting of a chemical compound, a protein, a peptide, a peptidomemetic, an antibody, a ribozyme, a small molecule chemical compound, a nucleic acid, a vector, and an antisense nucleic acid.

In one embodiment, the modulator of said composition increases the expression or activity of at least one selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

In one embodiment, the composition for treating or preventing a disease or disorder associated with misfolded protein or protein aggregates comprises an isolated peptide comprising one or more poly-D/E protein. In one embodiment, the isolated peptide further comprises a cell penetrating peptide (CPP) to allow for entry of the isolated peptide into a cell. In one embodiment, the CPP comprises the protein transduction domain of HIV tat. In one embodiment, the isolated peptide comprises a secretory signal peptide to direct secretion of the peptide to the extracellular environment.

In one embodiment, the composition for treating or preventing a disease or disorder associated with misfolded protein or protein aggregates comprises an isolated nucleic acid molecule encoding one or more poly-D/E protein. In one embodiment, the isolated nucleic acid molecule comprises an expression vector. In one embodiment, the expression vector comprises an adeno-associated viral (AAV) vector. In one embodiment, the AAV vectors comprises one or more selected from the group consisting of: AAV1, AAV2, AAV3, AAV4, AAV5, AAV6, AAV7, AAV8 and AAV9. In one embodiment, the isolated nucleic acid molecule encodes a peptide comprising a secretory signal peptide and one or more poly-D/E protein.

In one embodiment, the disease or disorder of the composition is one or more selected from the group consisting of: 1) a polyQ disorder; 2) a neurodegenerative disease or disorder selected from the group consisting of Spinocerebellar ataxia (SCA) Type 1 (SCA1), SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), a transmissible spongiform encephalopathy (prion disease), a synucleinopathy, dementia with Lewy bodies (DLB), multiple system atrophy (MSA), a tauopathy, and Frontotemporal lobar degeneration (FTLD); 3) a disease or disorder is selected from the group consisting of AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis; and 4) cancer associated with p53 aggregates.

In one embodiment, the present invention relates to a method of administering a composition comprising a modulator of one or more poly-D/E protein to a subject in need thereof, comprising contacting one or more cell or tissue of the subject with the composition of the present invention.

In one embodiment, the present invention relates to a method for treating or preventing a disease or disorder associated with misfolded protein or protein aggregates in a subject in need thereof, the method comprising administering to the subject a composition comprising a modulator of the expression or activity of one or more poly-D/E protein.

In one embodiment, the composition of the method comprises an isolated peptide comprising one or more poly-D/E protein selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

In one embodiment, the composition of the method comprises an isolated nucleic acid molecule encoding one or more poly-D/E protein.

In one embodiment, the method comprises administering the composition to at least one cell selected from the group consisting of: a neural cell, a glial cell, and a cancer cell.

In one embodiment, the disease or disorder of the method is one or more selected from the group consisting of: 1) a polyQ disorder; 2) a neurodegenerative disease or disorder selected from the group consisting of Spinocerebellar ataxia (SCA) Type 1 (SCA1), SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), a transmissible spongiform encephalopathy (prion disease),), synucleinopathy, dementia with Lewy bodies (DLB), multiple system atrophy (MSA), a tauopathy, and Frontotemporal lobar degeneration (FTLD); 3) a disease or disorder is selected from the group consisting of AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis; and 4) cancer associated with p53 aggregates.

In one embodiment, the present invention relates to a method for producing a recombinant protein comprising administering a modulator of one or more poly-D/E protein to cell modified to express a recombinant protein. In one embodiment, the modulator comprises one or more selected from the group consisting of: an isolated peptide comprising one or more poly-D/E protein and nucleic acid molecule encoding one or more poly-D/E protein.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1I, depicts exemplary results demonstrating that DAXX prevents protein misfolding and aggregation. FIGS. 1A-1B depict exemplary heat-induced luciferase inactivation (FIG. 1A, 5 nM) and aggregation (FIG. 1b, 200 nM) in the presence or absence of GST, DAXX, and Hsp70/Hsp40 at 200 nM (FIG. 1A) or at the indicated molar ratios (FIG. 1B). FIG. 1C depicts exemplary aggregation of Atxn1 82Q (50 nM) in the presence or absence of GST or DAXX (200 nM each). FIGS. 1D-1F depict exemplary fibrillization of α-Syn (70 μM) in the presence GST, Hsp70/Hsp40, HSPs (Hsp70/Hsp40-Hsp104^{A503S}) (200 nM each), and DAXX (100-400 nM) as assayed by ThT-binding (FIG. 1D), EM (FIG. 1E; red arrows, fibrils; blue arrows, large oligomers; scale bar, 100 nm), and sedimentation followed by dot blot for PE and SR aggregates and total α-Syn and by disuccinimidyl suberate (DSS) cross-linking (for soluble oligomers) (FIG. 1F). FIGS. 1G-1I depict exemplary fibrillization of Aβ42 monomers (10 μM) in the absence or presence of DAXX (50-600 nM) (FIGS. 1G and 1H), and viability of SH-SY5Y cells treated with Aβ42 pre-incubated with or without DAXX (FIG. 1I). An ATP-regeneration system was included with heat shock proteins but not DAXX. Data are mean±s.d. (n=4 for i, and 3 for the rest) and are representative of three independent experiments. *P<0.05; unpaired Student's t test.

FIG. 2, comprising FIG. 2A through FIG. 2I, depicts exemplary results demonstrating that DAXX dissolves protein aggregates and unfolds misfolded species. FIG. 2A depicts exemplary activity of heat-denatured luciferase (5 nM) treated with GST, DAXX, or HSPs (100 nM each). FIG. 2B depicts exemplary β-stand contents of native luciferase, or heat-denatured luciferase (1 μM each) treated with or without DAXX (0.1 and 0.5 μM). FIG. 2C depicts an exemplary sedimentation analysis of Atxn1 82Q aggregates (50 nM) treated with or without of GST or DAXX (200 nM). FIGS. 2D-2E depict exemplary ThT binding (FIG. 2D) and sedimentation (FIG. 2E) analyses of Aβ42 fibrils incubated with GST, HSPs (0.2 μM each), or DAXX (0.1, 0.2, and 0.4 μM). FIG. 2F depicts exemplary results showing that DAXX reduces the binding of monomeric misfolded LucD (3 μM) to ThT. FIG. 2G depicts exemplary results of the sensitivity to trypsin digestion of monomeric misfolded LucD (50 nM) upon incubation with DAXX, GST, or HSPs (100 nM each) for the indicated times. FIG. 2H depicts an exemplary kinetic analysis of LucD reactivation by DAXX or HSPs. FIG. 2I depicts an exemplary percentage of U2OS cells containing Atxn1-82Q inclusions of different sizes in the presence or absence of DAXX or Hsp70. Data are mean±s.d. (n=3) and are representative of two (FIG. 2B) or three (the rest) independent experiments. *P<0.05, **P<0.01, ***P<0.001; unpaired Student's t test for FIGS. 2B and 2D, two-way analysis of variance (ANOVA) for FIG. 2I.

FIG. 3, comprising FIG. 3A through FIG. 3J, depicts exemplary results demonstrating that other polyD/E proteins can function as molecular chaperones, disaggregases, and unfoldases. FIG. 3A depicts exemplary results showing the occurrence of each amino acid in DAXX-binding peptides relative to its occurrence in the peptide library (100%). FIG. 3B depicts exemplary reactivation of denatured luciferase (50 nM) by DAXX and HSPs (100 nM) in the presence of increasing salt concentration. FIGS. 3C-3D depict exemplary activity (FIG. 3C) and solubility (FIG. 3D) of denatured luciferase (5 nM) upon incubation with DAXX, DAXX ΔD/E, or DAXX D/E. n-Luc, native luciferase. FIGS. 3E-3F depict exemplary heat-induced aggregation of luciferase (200 nM) in the absence or presence of GST, ANP32A (FIG. 3E), or SET (FIG. 3F). FIGS. 3G and 31 depict exemplary prevention (FIG. 3G) and reversal (FIG. 3I) of Atxn1 82Q (50 nM) aggregation by SET and ANP32A (200 nM each). FIGS. 3H and 3J depict exemplary reactivation of heat-denatured luciferase (5 nM) (FIG. 3H) and misfolded LucD monomers (50 nM) (FIG. 3J) by SET and ANP32A (200 nM each). Data are mean±SD (n=3) and are representative of three independent experiments

FIG. 4, comprising FIG. 4A through FIG. 4F, depicts exemplary results demonstrating that DAXX maintains and restores the native conformation of p53 and MDM2. FIG. 4A depicts exemplary prevention of p53 (100 nM) aggregation by DAXX (200 nM). FIGS. 4B, 4D, and 4G depict exemplary solubilization of p53 (FIG. 4B), MDM2 (FIG. 4D), and p53^{R175H} (FIG. 4G) (100 nM each) aggregates by DAXX (200 nM). FIG. 4C depicts exemplary immunoprecipitation (IP) of native p53, or denatured p53 incubated with GST or DAXX, with PAb1620, PAb240, or DO1 (pan-p53 antibody). RM, proteins remained in supernatants. FIG. 4E depicts exemplary ubiquitination of p53 (20 nM) by MDM2 (45 nM) in the presence or absence of DAXX (100 nM), or by denatured MDM2 (45 nM) pre-incubated with or without DAXX. FIG. 4F depicts exemplary prevention of p53 and p53^{R175H} (5 μM) fibrillization by DAXX (5 μM). FIGS. 4H and 4I depicts representative images of U2OS cells transfected with p53^{R175H} or p53^{R175H} plus DAXX (FIG. 4H), and the percentage of cells containing p53^{R175H} puncta (FIG. 4I). Scar bar: 10 μm. FIG. 4J depicts exemplary p53^{R280K} aggregates in control and DAXX-knockdown MDA-MB-231 cells. FIG. 4K depicts exemplary fluorescence intensity of anti-p53 (DO-1) and anti-pre-fibril oligomers (A11) staining in control MDA-MB-231 cells or MDA-MB-231 cells transfected with DAXX siRNAs and/or Flag-DAXX. FIG. 4L depicts the exemplary number and size of soft-agar colonies formed by control and DAXX-overexpressing MDA-MB-231 cells. Data are mean±s.d. and are representative of two (FIG. 4J) or three (the rest) independent experiments. *P<0.05, **P<0.01, ***P<0.001, ns, not significant; unpaired Student's t test.

FIG. 5, comprising FIG. 5A through FIG. 5J, depicts the results of example experiments demonstrating purification of recombinant DAXX proteins and their ability to prevent luciferase misfolding and aggregation. FIG. 5A depicts a scheme for purifying DAXX-6×His from bacteria BL21 (DE3) and insect Sf9 cells. To generate the full-length protein, DAXX was fused to GST at the N terminus with a TEV protease cleavage site inserted in between GST and DAXX, and to 6×His at the C terminus. The fusion protein was first purified with glutathione resins. Beads-bound GST-DAXX-6×His protein was treated with TEV protease to released DAXX-6×His, which was subsequently purified with Ni-NTA resins. After elution with imidazole, DAXX-6×His was further purified with ion-exchange (IEX) and gel filtration columns, and concentrated as needed. FIGS. 5B and C depict exemplary DAXX-6×His purified from bacteria BL21 (DE3) cells (FIG. 5B) and insect Sf9 cells (FIG. 5C), analyzed by SDS-PAGE and Coomassie blue staining. Bovine serum albumin (BSA) was used as a protein standard (FIG. 5C). Mass spectrometry analysis indicated that the vast majority of species in the minor bands of the DAXX preps were derived from DAXX. FIG. 5D depicts a scheme for purifying Flag-DAXX from HEK293T cells. Flag-DAXX was transiently transfected in HEK293T cells and purified using anti-Flag M2 beads. After elution with 3×Flag peptides, Flag-DAXX was further purified with ion-exchange and gel filtration columns, and concentrated as needed. FIG. 5E depicts exemplary Flag-DAXX purified from HEK293T cells, analyzed by SDS-PAGE and Coomassie blue staining. Mass spectrometry analysis indicated that the vast majority of species in the minor bands of the DAXX preps were derived from DAXX. FIGS. 5F-5H depict exemplary results showing that DAXX proteins purified from bacteria, insect cells, and mammalian cells protect luciferase from heat-induced inactivation and aggregation. Luciferase (FIGS. 5F and 5G, 5 nM; FIG. 5H, 200 nM) was heated at 42° C. in the presence of indicated concentrations of GST, DAXX-6×His (from bacteria), or Hsp70 (plus Hsp40 at a half concentration, same below) for 1 min (FIG. 5F), or in the presence or absence of GST, DAXX-6×His (from bacteria), DAXX-6×His (from insect cells), or Flag-DAXX (200 nM each) for the indicated times (FIGS. 5G and H). Shown are luciferase activity relative to the native protein (FIGS. 5F and 5G) and relatively turbidity measured at OD₆₀₀ (FIG. 5H). The DAXX protein purified from HEK293T cells appeared to be more active than those purified from bacteria and insect cells. FIGS. 5I and 5J depict exemplary protective activity of DAXX for a higher amount of luciferase. Luciferase (50 nM) was heated at 42° C. in the presence of the indicated concentrations of GST, DAXX-6×His (from sf9 cells), or Hsp70 for 1 min (FIG. 5I), or in the presence of absence of GST, DAXX-6×His (Sf9 cells), or Hsp70/Hsp40 (200 nM each) for the indicated times. Luciferase activity was normalized to native protein. RT-CTRL, control luciferase sample kept at room temperature. Assays in FIGS. 5B, 5C, and 5E have been performed three times with similar results. Numerical data are mean±SD (n=3) and are representative of three (FIGS. 5F and 5J) or two (FIGS. 5G-5I) independent experiments. *P<0.05, **P<0.01, ***P<0.001; ns, not significant, unpaired Student's t test.

FIG. 6, comprising FIG. 6A through FIG. 6L, depicts the results of example experiments demonstrating that DAXX prevents α-Syn and Aβ42 aggregation, acts independently of ATP, and likely exists as a monomer. FIGS. 6A and 6B depict exemplary PFFs-induced aggregation of soluble α-Syn monomers and its inhibition by DAXX. α-Syn monomers (13.3 μM) were incubated alone, together with in α-Syn PFFs (133 nM) (FIG. 6A), or together with in α-Syn PFFs (133 nM) in the presence of GST (0.2 μM), HSPs (0.2 μM Hsp70, 0.1 μM Hsp40, and 0.4 μM Hsp104^{A503S}), and DAXX-6×His (from Sf9 cells, 0.1, 0.2, and 0.4 μM) (FIG. 6B). Aggregation was monitored by real-time quaking-induced conversion (RT-QuIC) assay. FIGS. 6C-6E depict exemplary results showing that DAXX suppresses Aβ42 fibrillization for a prolonged incubation, during which DAXX itself did not form fibrils or other sedimentable aggregates. Aβ42 monomers (10 μM) and DAXX-6×His (from Sf9 cells, 0.05, 0.1, 0.2, 0.4, and 0.6 μM) were incubated alone (FIGS. 6C and 6E) or together (FIG. 6D) at 37° C. for 120 h. Formation of fibrils was analyzed by ThT fluorescence assay (FIGS. 6C and 6D). Solubility of DAXX was analyzed by sedimentation assay (FIG. 6E). FIGS. 6F and 6G depict exemplary results showing that DAXX blocks Aβ42 monomers to form PFFs that accelerate aggregation of fresh Aβ42 monomers and Aβ42 PFFs-induced aggregation of fresh Aβ42 monomers. Aβ42 monomers (10 μM) were incubated at 37° C. alone, together Aβ42 PFFs (6 nM) (FIGS. 6F and 6G), Aβ42 (6 nM) preincubated with DAXX-6×His (from Sf9 cells) at a 100:1 molar ratio (Aβ42/DAXX), Aβ42/DAXX plus DAXX-6×His (0.6 μM) (DAXX) (FIG. 6F), or Aβ42 PFFs (6 nM) in the presence of DAXX (FIG. 6G). Formation of fibrils was analyzed by ThT fluorescence assay. Assays in FIGS. 6F and 6G were done at the same time. FIGS. 6H and 6I depict exemplary results showing that the chaperone activity of DAXX is not affected by the addition of ATP or the treatment of apyrase. Luciferase (0.2 μM) was heated at 42° C. in presence of GST (0.2 μM) (FIGS. 6H and 6I), DAXX-6×His (insect cells, 0.2 μM) with or without ATP (5 mM ATP-Mg²⁺ plus an ATP-regeneration system) and apyrase as indicated (FIG. 6H), or Hsp70/Hsp40 (0.2 and 0.1 μM, respectively) with or without apyrase (FIG. 6I). Aggregation formation was monitored by OD at 600 nm. FIG. 6J depicts exemplary results showing that DAXX does not bind to ATP. Recombinant DAXX-6×His and Hsp70 were incubated with agarose beads conjugated without ATP (−) or with ATP via the phosphate moiety (AP-ATP), ribose moiety (EDA-ATP), or the adenine base at different positions (6AH-ATP and 8AH-ATP). The input and pulldown samples were analyzed by western blot. FIG. 6K depicts exemplary results showing that DAXX exists as a homogeneous species of relatively low molecular weights. Recombinant Flag-DAXX protein was analyzed by Superdex 200 10/300 GL column. Proteins standards (in kDa) are indicated. FIG. 6L depicts exemplary results showing that DAXX likely exists as a monomer. Recombinant Flag-DAXX (1 μM) was crosslinked with indicated concentration of DSS at 25° C. for 30 min and analyzed by Western blot. Flag-p53 (1 μM), which is expected to be a tetramer, was used as control. Similar results were obtained for DAXX-6×His. Assays have been performed three (FIGS. 6B-6E, 6K, and 6L) or two (FIGS. 6A and 6H-6J) times with similar results. Numerical data are mean±s.d. (n=3) and are representative of three independent experiments (FIGS. 6F and 6G).

FIG. 7, comprising FIG. 7A through FIG. 7P, depicts the results of example experiments demonstrating that DAXX can dissolve small luciferase aggregates, but not large luciferase aggregates or α-Syn fibrils. FIGS. 7A-7C depict exemplary results showing that DAXX dissolves and reactivates heat-denatured luciferase aggregates. Heat-denatured luciferase (5 nM) was incubated at 25° C. with GST (100 nM), DAXX-6×His (from E. coli, 100 nM), and HSPs (100 nM Hsp70, 50 nM Hsp40, and 200 nM Hsp104^{A503S}) for the indicated times (FIG. 7A), or with the indicated amounts of GST or DAXX-6×His for 90 min (FIGS. 7B and 7C). Shown are luciferase activity relative to that of native luciferase (FIG. 7B), and the amount of luciferase in the SN after sedimentation (FIGS. 7A and 7C). FIG. 7D depicts exemplary results showing the disaggregase activity of DAXX proteins purified from different sources. Relative activity of heat-denatured luciferase (5 nM) that was incubated at 25° C. for 90 min with increasing concentrations of DAXX purified from the indicated sources. FIGS. 7E and 7F depict exemplary results showing the disaggregase activity of DAXX for a higher amount of denatured luciferase. Relative activity of heat-denatured luciferase (50 nM) that was incubated at 25° C. with the increasing molar ratios of DAXX-6×His (from Sf9 cells) for the indicated times (FIG. 7E) or 90 min (FIG. 7F). FIG. 7G depicts exemplary results showing that DAXX achieves the maximal recovery of luciferase activity at five-fold excess. Heat-denatured luciferase (0.1, 0.2, 0.5, 1, and 2 μM) was incubated with DAXX-6×His (0.1 μM) at 25° C. for 90 min. Luciferase activity is relative to that of native luciferase. FIG. 7H depicts exemplary results showing that DAXX restores the native conformation to denatured luciferase. Native or heat-denatured luciferase (1 μM) incubated alone or together with GST (1 μM) or DAXX-6×His (0.1×: 0.1 μM; 0.5×: 0.5 μM) for 90 min were examined by circular dichroism (CD) spectroscopy. Data was analyzed by CAPITO, with the percentages of β-strand shown in FIG. 2B. FIG. 7I depicts exemplary results showing the different sizes of luciferase aggregates generated by heat and urea treatments. Luciferase (1 μM) denatured by heat or urea was fractionated on Superdex 200 10/300 GL column. Fractions were analyzed by Western blot and the relative abundance of luciferase is indicated. FIG. 7J depicts exemplary results showing that DAXX cannot reactive urea-denatured luciferase. Relative activity of urea-denatured luciferase (5 nM) that was incubated with GST (0.2μ), DAXX (0.2 and 1 μM), or HSPs (0.2 μM Hsp70, 0.1 μM Hsp40, and 0.4 μM Hsp104^{A503S}) at 25° C. for 90 min. FIGS. 7K and 7L depict exemplary results of luciferase denatured by heat (FIG. 7K) or urea (FIG. 7L), fractionated on gel filtration chromatography. Fractions in the range of 44 to 2,000 kDa were incubated with buffer, lysozyme (0.1 μM), DAXX-6×His (0.1 μM), or Hsp70/Hsp40-Hsp104^{A503S} (0.1, 0.05, and 0.2 μM, respectively) at 25° C. for 90 min, and luciferase activity was determined. FIGS. 7M-7P depict exemplary results showing that DAXX is unable to dissolve α-Syn fibrils. Preformed α-Syn fibrils (0.2 μM) were treated with GST (0.2 μM) (FIGS. 7M-7P), DAXX-6×His at the indicated molar ratios (FIGS. 7M and 7N) or at 0.2 μM (FIGS. 7O and 7P), HSPs (0.2 μM Hsp70, 0.1 μM Hsp40, and 0.4 μM Hsp104^{A503S} plus ATP and an ATP regeneration system) (FIGS. 7M-7P), or both DAXX and HSPs (FIGS. 7O and 7P). Reaction mixtures were analyzed by dot blot (FIGS. 7M and 7O), and soluble α-Syn relative to total α-Syn was quantified (FIGS. 7N and 7P) (n=3). Assays in panels FIGS. 7A, 7C, 7hH, 7I, and 7K-7P have been performed three times with similar results. Numerical data are mean±s.d. (n=3) and are representative of three independent experiments (FIGS. 7B, 7D-7G, and 7J). **P<0.01, ***P<0.001, ns, not significant; unpaired Student's t test.

FIG. 8, comprising FIG. 8A through FIG. 8J, depicts the results of example experiments demonstrating that DAXX unfolds misfolded LucD and protects against protein aggregation and oligomerization in cells. FIG. 8A, A schematic representation of compact misfolded LucD monomers, the unfolded intermediates, and the native conformers, as well as their sensitivity to brief trypsin digestion. FIG. 8B, DAXX changes trypsin sensitivity of LucD. LucD (50 nM) was incubated with DAXX-6×His (100 nM), GST (100 nM), or HSPs (100 nM Hsp70, 50 nM Hsp40, and 200 nM Hsp104^{A503S}) at 25° C. At indicated time points, aliquots of luciferase were incubated with 2.5 μM trypsin at 22° C. for 2 min and were analyzed by Western blot. Shown is luciferase band intensity. A representative western blot is presented in FIG. 2G. FIG. 8C, DAXX increases the enzymatic activity of LucD. Misfolded LucD monomers (50 nM) were incubated with GST (100 nM) or DAXX-6×His (100 nM) for indicated durations and assayed for luciferase activity. FIG. 8D, DAXX elevates the levels of nLucDM, but not nLuc, in cells. nLuc or nLucDM was transfected together with empty vector (EV) or Flag-DAXX in HEK293T cells. Cell lysates were analyzed by Western blot 24 h after transfection. FIGS. 8E and 8F, DAXX reduces aggregation, but not expression, of Atxn1 82Q in cells. U2OS cells transfected with HA-Atxn1-82Q together with EV, Flag-DAXX (FIGS. 8E-8F), or GFP-Hsp70 (FIG. 8F) were analyzed by Western blot (FIG. 8E), or immunofluorescence with anti-HA (red) and anti-Flag (green) antibodies (FIG. 8F; scale bar, 10 μm). Quantification of the percentage of cells containing different sizes Atxn1-82Q inclusions is shown in FIG. 2I. FIG. 8G, Schematic representation of bimolecular fluorescence complementation (BiFC) assay based on Venus, an improved version of yellow fluorescent protein (YFP). FIGS. 8H-8J, DAXX inhibits α-Syn oligomerization in cells. HEK293T cells were transfected with VIS and SV2 individually, or together with empty vector (EV) or DAXX. Cells were analyzed by Western blot for protein expression (FIG. 8H) and by fluorescence microscopy for BiFC signals and Flag-DAXX expression (FIG. 8I; Scale bars, 100 μm), with the quantification of BiFC signals shown in (FIG. 8J). Assay in panels FIGS. 8D-8F 8H, and 8I have been performed two times with similar results. Numerical data are mean±s.d. (n=3) and are representative of three independent experiments (FIGS. 8B, 8C, and 8J). *P<0.05, **P<0.01, ***P<0.001; ns, not significant, unpaired Student's t test.

FIG. 9, comprising FIG. 9A through FIG. 9H, depicts the results of example experiments demonstrating that DAXX binds to misfolded proteins and depends on the polyD/E region for its activity. FIG. 9A depicts exemplary results showing that the DAXX-luciferase interaction is increased upon heat shock. HEK293T cells were transfected with HA-DAXX and Flag-nLuc as indicated, and treated with or without heat shock. Cell lysates were immunoprecipitated (IP) with anti-Flag mAb (M2) beads. Input and IP samples were analyzed by Western blot. FIG. 9B depicts exemplary results showing that DAXX preferentially binds to heat-denatured luciferase. GST or GST-DAXX (100 nM each) was incubated with native (N) or heat-denatured (D) 6×His-luciferase immobilized on Ni-NTA agarose. The input and pulldown samples were analyzed by Western blot. FIG. 9C depicts exemplary results of binding of DAXX to cellulose-bound peptide scans derived from luciferase, p53, MDM2, H3.3, H4, and DAXX. Each peptide contained thirteen amino acid residues that overlapped adjacent peptides by ten. FIG. 9D depicts the protein sequence of DAXX. The poly D/E region is marked in red color, and the four continuous runs of Asp/Glu (with 14, 11, 7, and 5 residues, respectively) are underlined. FIG. 9E depicts a schematic presentation of full-length DAXX and its mutants. DAXX D/E was fused to GST at the N terminus for protein stabilization. FIG. 9F depicts exemplary results showing that DAXX ΔD/E and DAXX D/E lack unfoldase activity. Misfolded LucD monomers (50 nM) were incubated with DAXX ΔD/E or DAXX D/(100 nM each) for the indicated times and assayed for luciferase activity (Mean±S.D., n=3). FIGS. 9G and 9H depict exemplary results showing that DAXX ΔD/E and DAXX D/E remain soluble during heat treatment. Recombinant DAXX ΔD/E (FIG. 9G) and D/E (FIG. 9H) proteins were heated at 42° C. for the indicated durations. Luciferase (200 nM) was used as a positive control. Aggregation formation was monitored by OD₆₀₀ and normalized to the luciferase control. Assays in FIGS. 9A-9C have been performed two times with similar results. Numerical data are mean or mean±s.d. (n=3) and are representative of two independent experiments (FIGS. 9F-9H).

FIG. 10, comprising FIG. 10A through FIG. 10L, depicts the results of example experiments demonstrating the role of other polyD/E proteins in protein folding. FIG. 10A depicts sequences of human SET and ANP32A proteins. The poly D/E region is shaded, and the continuous runs of Asp/Glu are underlined. FIGS. 10B and 10C depict exemplary results showing that ANP32A and SET does not block α-Syn fibrillization. α-Syn monomers (70 μM) were incubated with SET or ANP32A (0.4 μM each) for 7 days. Samples were analyzed by electron microscopy (FIG. 10B) and ThT staining (FIG. 10C). Scale bar, 100 nm. FIG. 10D depicts exemplary results showing that SET and ANP32A are unable to solubilize urea-denatured luciferase. Urea-denatured luciferase (5 nM) was incubated with GST, SET, ANP32A (0.2 μM each), or HSPs (0.2 μM Hsp70, 0.1 μM Hsp40, and 0.4 μM Hsp104^{A503S}) at 25° C. for 90 min. Activity relative to that of the native control are shown. FIGS. 10E and 10F depict exemplary results showing the unfoldase activity of ANP32A and SET. Misfolded LucD (50 nM) was incubated with GST, SET, or ANP32A (200 nM each) at 25° C. At indicated time points, aliquots of refolding luciferase were incubated with 2.5 μM trypsin at 22° C. for 2 min, denatured in SDS sample buffer, and analyzed by Western blot (FIG. 10E), with the quantification showed in (FIG. 10F). FIGS. 10G and 10H depict a schematic presentation of SET and its deletion mutants (FIG. 10G), and the numbers of Asp (D) and Glu (E) in each mutant (FIG. 10H). FIG. 10I depicts exemplary results heat-inactivated luciferase (5 nM), incubated at 25° C. with SET or its deletion mutants (200 nM each) for 90 min. Activity relative to that of native luciferase are shown. FIG. 10J depicts the number of polyD/E proteins different species. FIGS. 10K-10L depict exemplary results showing a gene ontology analysis of polyD/E proteins in humans. Proteins are classified into pie chart based on their molecular functions (FIG. 10K) and protein classes (FIG. 10L). Assays have been performed two (FIG. 10B) or three (FIG. 10E) times with similar results. Numerical data are mean±s.d. (n=3) and are representative of two independent experiments (FIGS. 10C, 10D, 10F, and 10I). **P<0.01, ns, not significant; unpaired Student's t test.

FIG. 11, comprising FIG. 11A through FIG. 11O, depicts the results of example experiments demonstrating that DAXX maintains the native conformation of both p53 and MDM2. FIG. 11A depicts exemplary results showing that DAXX abrogates p53 fibrillization. Recombinant wild-type p53 and DAXX-6×His proteins (5 μM each) were incubated alone or together at 37° C. for 2 h in the presence of ThT (25 μM). Formation of amyloid fibrils was assayed by ThT. FIGS. 11B and 11C depict exemplary results showing that DAXX ΔD/E and DAXX D/E cannot protect p53 from aggregation. Native p53 (n-p53) (FIG. 11B), or denatured p53 aggregates (d-p53) (FIG. 11C), (100 nM each) was incubated with GST, Flag-DAXX, DAXX D/E, or DAXX ΔD/E (200 nM each) at 37° C. (FIG. 11B) or at 25° C. (FIG. 11C) for the indicated times. Samples were partitioned into supernatant (SN, soluble) and pellet (PE, insoluble) fractions via sedimentation, and analyzed by Western blot. Same as FIGS. 4A and 4B except that DAXX D/E and DAXX ΔD/E samples are included. FIGS. 11D, 11E, 11G, and 11H depict exemplary results showing that DAXX restores the native conformation to denatured p53 and MDM2. Native p53 (FIG. 11D) or MDM2 (n-MDM2, FIG. 11G), or denatured p53 (FIG. 11E) or MDM2 (d-MDM2, FIG. 11H), (1 μM each) was incubated alone or together with GST or DAXX-6×His (0.5, 1, 2 μM, from Sf9 cells) at the indicated molar ratios for 3 h and analyzed by thermal shift assay. The transition of the unfolding curve represents the temperature at which the protein unfolding occurs (Tₘ). FIG. 11F depicts exemplary results showing that DAXX dissolves preformed MDM2 aggregates. d-MDM2 (100 nM) was incubated with Flag-DAXX (200 nM) at 25° C. for the indicated times. Supernatant (SN, soluble) and pellet (PE, insoluble) fractions after sedimentation were analyzed by Western blot. FIG. 11I depicts exemplary results showing that DAXX enhances MDM2-mediated p53 ubiquitination. Native p53 (20 nM) was incubated with native MDM2 (45 nM) in the presence or absence of DAXX (20 or 100 nM) at 37° C. for 1.5 h. E1, E2 and His-ubiquitin (His-Ub) were then added for in vitro ubiquitination assay. The reaction mixtures were analyzed by Western blot. FIG. 11J depicts exemplary results showing native MDM2-mediated ubiquitination of native p53 (20 nM) in the presence or absence of Flag-DAXX (100 nM), or of denatured p53 (20 nM) pre-incubated with or without Flag-DAXX (100 nM) for 3 h at 25° C. FIGS. 11K and 11L depict exemplary results showing that DAXX reduces p53 levels in cells, but does not alter the largely diffuse nuclear localization pattern of p53. Flag-p53 was transfected into U2OS cells together with empty vector (CTRL) or DAXX. Cells were analyzed by immunofluorescence (FIG. 11K) and western blot (FIG. 11L). FIGS. 11M-11O depict exemplary results of H1299 cells inducibly expressing wild-type p53 or p53^{R175H}, transfected with control vector (−) or HA-DAXX. Upon induction of p53 expression by Dox (1 μg/mL), cells were analyzed for protein levels by Western blot with relative p53/GAPDH ratios indicated (FIG. 11M) and for mRNA levels of p53 (FIG. 11N) and p53 target genes (FIG. 11O) by qRT-PCR. Scar bar: 10 μm. Assays in panels have been performed two (FIGS. 11D, 11E, 11G, 11H, and 11K-11M) or three (FIGS. 11B, 11C, 11F, 11I, and 11J) times with similar results. Numerical data are mean±s.d. (n=3) and are representative of two independent experiments (FIGS. 11A, 11N, and 11O). *P<0.05, **P<0.01, ns, not significant; unpaired Student's t test.

FIG. 12, comprising FIG. 12A through FIG. 12L, depicts the results of example experiments demonstrating that DAXX restores native conformation and function of mutant p53. FIG. 12A depicts exemplary results showing that DAXX prevents p53^{R175H} aggregation. p53^{R175H} protein (100 nM) was incubated with GST or Flag-DAXX (200 nM each) at 37° C. for the indicated times. SN and PE fractions were analyzed by Western blot. FIG. 12B depicts exemplary results showing that DAXX blocks p53^{R175H} PFFs-induced fibrillization of p53. Wild-type p53 (5 μM) was incubated with or without p53^{R175H} PFFs and DAXX as indicated. Fibril formation was assayed by ThT binding. FIG. 12C depicts exemplary results showing that DAXX reduces p53^{R175H} aggregates in cells. Flag-p53^{R175H} was transfected into the U2OS cells together with empty vector (CTRL) or HA-DAXX. Cells were analyzed by immunofluorescence. Scar bar: 10 μm. Part of the images are also shown in FIG. 4I. FIG. 12D depicts exemplary results showing that DAXX partially restores the function of mutant p53. H1299 cells inducibly expressing p53^{R175H} were transfected with control vector (−) or HA-DAXX. Upon induction of p53 expression by Dox (1 μg/mL), cells were analyzed for the expression of p53 target genes by RT-PCR. FIGS. 12E-12G depict exemplary results showing the effect of DAXX on aggregation of endogenous mutant p53. MDA-MB-231 cells were transduced with lentiviral vectors expressing control or DAXX shRNA (FIGS. 12E and 12F), or transfected with control siRNA, DAXX siRNA, and/or an siRNA-resistant form of DAXX (Flag-DAXX) as indicated (FIG. 12G). Cells were immunostained with anti-p53 (DO-1) and anti-fibrillar oligomer (A11) antibodies (FIGS. 12E and 12G) and quantified (FIG. 12F). Scar bar: 50 μm. FIGS. 12H-12J depict exemplary results showing that knocking down DAXX enhances growth and tumorigenicity of MDA-MB-231 cells. Control and DAXX-knockdown MDA-MB-231 cells were assayed for adherent proliferation, protein expression (FIG. 12H), and soft-agar colony formation (21 days), with number and sizes of colonies (FIG. 12I) and representative images of colonies (FIG. 12J) shown. FIGS. 12K and 12L depict exemplary results showing that overexpressing DAXX inhibits growth and tumorigenicity of MDA-MB-231 cells. MDA-MB-231 transduced with pBabe or pBabe-Flag-DAXX were assay for adherent proliferation for 5 days (FIG. 12K) and soft-agar colony formation (21 days), with representative images of colonies shown (FIG. 12L). Assays have been performed two (FIG. 12A) or three (FIGS. 12C, 12E, and 12G) times with similar results. Numerical data are mean±s.d. (n=3 for FIGS. 12B and 12D, and 6 for FIGS. 12I, 12H, and 12K) and are representative of three independent experiments. *P<0.05, **P<0.01, ***P<0.001, ns, not significant; unpaired Student's t test

FIG. 13 depicts a tables of human poly-D/E proteins containing 35 or more D or E residues in any 50-residue window. Poly-D/E-containing proteins from humans are listed with UniProt ID (first column), protein symbol (second column), and protein name (third column). The number of D and E residues inside the window are list in the fourth and fifth columns, respectively. Sequences of the poly-D/E region are listed in the sixth column with D/E shaded. A list of gene ontology (GO) terms is listed in the last column. All proteins listed were clustered into different categories depending on the protein class or molecular function (see FIGS. 10K and 10L).

FIG. 14, comprising FIG. 14A through FIG. 14D, depicts representative results of the action of DAXX on Tau fibrils. FIG. 14A depicts representative fluorescence results of Tau fibrillization and inhibition of fibrillization by DAXX. FIG. 14B depicts representative imaging of Western blot and dot blot analysis detecting soluble Tau, demonstrating reduced fibrillization. FIG. 14C depicts representative fluorescence results of Tau aggregates being disaggregated by Tau. FIG. 14D depicts representative imaging of Western blot and dot blot analysis demonstrating DAXX dissolves Tau aggregates.

FIG. 15, comprising FIG. 15A through FIG. 15D, depicts representative effects of DAXX on Tau aggregation in cells. FIG. 15A depicts representative imaging of GFP-Tau in the presence of DAXX, demonstrating reduced aggregation. FIG. 15B depicts a representative schematic of a bimolecular fluorescence complementation (BiFC) assay. FIG. 15C depicts representative fluorescence imaging of a BiFC assay with DAXX. FIG. 15D depicts quantification of the fluorescence in FIG. 15C and representative imaging of western blotting for Tau, demonstrating DAXX blocks formation of Tau oligomers but does not target Tau for degradation.

FIG. 16, comprising FIG. 16A through FIG. 16I, depicts representative data demonstrating DAXX inhibits polyQ, FUS, and TDP-43 aggregation. FIG. 16A depicts representative imaging of a Western blot demonstrating DAXX inhibits aggregation of huntingtin. FIG. 16B depicts representative imaging of a Western blots demonstrating DAXX inhibits aggregation of FUS. FIG. 16C depicts representative fluorescence imaging of TDP inclusions in cells co-expressing TDP-43 and DAXX. FIG. 16D depicts quantification of the number of folds seen in the TDP inclusions when TDP-43 is expressed with full-length DAXX, a truncated DAXX lacking the histone binding domain, and DAXX lacking the polyD/E region. FIG. 16E depicts quantification of size of folds seen in the TDP inclusions when TDP-43 is expressed with full-length DAXX, a truncated DAXX lacking the histone binding domain, and DAXX lacking the polyD/E region. FIG. 16F depicts the sequence of DAXX and the polyD/E region (amino acids 449-499). FIG. 16G depicts schematic representations of full length DAXX, a truncated DAXX lacking a full histone binding domain but maintaining the polyD/E region, and a DAXX lacking the polyD/E region. FIG. 16 H depicts representative imaging of a Western blot demonstrating that DAXX expression reduces the formation of TDP-43 aggregates in a Q331K mutant of TDP-43. FIG. 16I depicts representative imaging of a Western blot demonstrating that DAXX expression reduces the formation of TDP-43 aggregates in a M337K mutant of TDP-43.

### DETAILED DESCRIPTION

The present invention is related to the discovery of the role of poly-D/E acidic region-containing proteins as molecular chaperones, disaggregases and unfoldases for misfolded proteins, which play a role in the pathology of a variety of neurodegenerative disorders and cancers.

In one aspect, the present invention provides compositions and methods to treat or prevent a disease or disorder associated with misfolded protein or protein aggregates. It is demonstrated herein that poly-D/E proteins have roles as molecular chaperones, in disaggregating protein aggregates and in unfolding stable misfolded monomers. Thus, in certain aspects, the present invention can be used to eliminate intracellular or extracellular misfolded proteins, protein aggregates, or protein inclusions.

For example, in certain embodiments, the invention provides compositions and methods to treat or prevent a neurodegenerative disorder in a subject in need thereof. For example, in certain embodiments, the invention provides compositions and methods for the treatment or prevention of neurodegenerative disorders that are poly-glutamine (polyQ) disorders, where repeats of the CAG codon encode proteins with polyglutamine tracts that can result in misfolded protein aggregates. Exemplary polyQ disorders include, but are not limited to Spinocerebellar ataxia (SCA) Type 1 (SCA1), SCA2, SCA3, SCA6, SCA7, SCA17, Huntington's disease, and Dentatorubral-pallidoluysian atrophy (DRPLA).

In some embodiments, the invention provides compositions and methods for the treatment of disorders associated with misfolded proteins or protein aggregates. For example, in certain embodiments, the compositions and methods are used for the treatment of diseases and disorders associated with misfolded proteins and/or protein aggregates of amyloid-beta, alpha-synuclein, tau, prions, SOD1, TDP-43, FUS, p53, p53 mutants, or proteins associated with polyglutamine repeats, such as huntingtin and ataxins.

Exemplary neurodegenerative diseases associated with misfolded proteins or protein aggregates include, but are not limited to, Alzheimer's disease, Parkinson's disease, amyotrophic lateral sclerosis (ALS), transmissible spongiform encephalopathies (prion disease), synucleinopathies, dementia with Lewy bodies (DLB), multiple system atrophy (MSA), tauopathies, and Frontotemporal lobar degeneration (FTLD). However, the present invention is not limited to the treatment or prevention of neurodegenerative disorders. Rather, the invention encompasses the treatment or prevention of any disease or disorder associated with a misfolded protein or protein aggregate. Other such diseases and disorders include, but is not limited to AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis.

In some embodiments, the invention encompasses the treatment or prevention of cancer associated with p53 aggregates.

In one aspect, the invention encompasses the use of one or more poly-D/E protein to stabilize a misfolded protein. In certain aspects, stabilization of a functional misfolded protein via one or more poly-D/E protein described herein can treat or prevent a disease or disorder associated with the misfolded protein. For example, in one embodiment, stabilization of mutant cystic fibrosis transmembrane conductance regulator (CFTR), via one or more poly-D/E protein described herein, would allow mutant CFTR to function instead of being degraded. It is envisioned that using poly-D/E proteins to stabilize misfolded proteins can be used to treat cystic fibrosis and other diseases associated with degradation of partially functional proteins. Stabilization of proteins, via one or more poly-D/E protein described herein, can be used to treat any disease or disorder associated with degradation of functional mutant protein, including but not limited to cystic fibrosis and lysosomal storage diseases such as Gaucher's disease and Fabry's disease.

In one aspect, the present invention provides compositions and methods to increase the expression, activity, or both of a poly-D/E protein. In certain embodiments, the composition comprises a nucleic acid molecule, expression vector, protein, peptide, small molecule, or the like, which increases the expression, activity, or both of one or more poly-D/E protein.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “cell therapy” refers to the administration of viable cells to a subject as a therapeutic to treat or prevent one or more disease or disorder. The cells can be unmanipulated or manipulated, such as cells genetically engineered to overexpressed a therapeutic protein of interest. The cells for use in cell therapy can be xenogeneic, allogeneic, syngeneic, or autologous.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, a “modulator of one or more poly-D/E protein” is a compound that modifies the expression, activity or biological function of the poly-D/E protein as compared to the expression, activity or biological function of the poly-D/E protein in the absence of the modulator.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention provides compositions and methods to treat or prevent a disease or disorder associated with misfolded proteins or protein aggregates. For example, the present invention provides compositions and methods to increase the recognition and elimination of misfolded proteins. In certain embodiments, the invention assists in the folding of proteins. In certain embodiments, the invention provides for the disaggregation and refolding of protein aggregates or inclusions. The present invention can thus be used to treat or prevent misfolded proteins, protein aggregates, or protein inclusions, both intracellularly or extracellularly.

The present invention is related to the discovery of the role of poly-D/E acidic region-containing proteins as molecular chaperones, disaggregases and unfoldases for misfolded proteins, which play a role in the pathology of a variety of neurodegenerative disorders and cancer.

### Compositions

### Modulators

In some embodiments, the present invention includes compositions to prevent or remove misfolded protein, protein aggregates, or a combination thereof. In some embodiments, the composition is for use in preventing or treating one or more disease or disorder associated with misfolded protein, protein aggregates or a combination thereof. In some embodiments the composition comprises a modulator to increase the function, level, or activity of a gene or gene product that promotes molecular chaperone activity, disaggregase activity, unfoldase activity or a combination thereof. In some embodiments the composition comprises a modulator to decrease the function, level, or activity of a gene or gene product that promotes protein misfolding, protein aggregation, or a combination thereof. In some embodiments, the composition comprises: 1) a modulator to increase the function, level, or activity of a gene or gene product that promotes molecular chaperone activity, disaggregase activity, unfoldase activity or a combination thereof; and 2) a modulator to decrease the function, level, or activity of a gene or gene product that promotes protein misfolding, protein aggregation, or a combination thereof.

It will be understood by one skilled in the art, based upon the disclosure provided herein, that modulating a gene, or gene product, encompasses modulating the level or activity of a gene, or gene product, including, but not limited to, modulating the transcription, translation, splicing, degradation, enzymatic activity, binding activity, or combinations thereof. Thus, modulating the level or activity of a gene, or gene product, includes, but is not limited to, modulating transcription, translation, degradation, splicing, or combinations thereof, of a nucleic acid; and it also includes modulating any activity of polypeptide gene product as well.

In one embodiment, the modulator increases the expression or activity of a gene or gene product by increasing production of the gene or gene product, for example by modulating transcription of the gene or translation of the gene product. In one embodiment, the modulator increases the expression or activity of a gene or gene product by providing exogenous gene or gene product. For example, in certain embodiments, the modulator comprises an isolated nucleic acid encoding one or more poly-D/E protein. In certain embodiments, the modulator comprises an isolated peptide comprising a poly-D/E protein. In one embodiment, the composition comprises one or more modulator comprising one or more isolated peptide comprising one or more poly-D/E protein. In one embodiment, the modulator increases the expression or activity of a gene or gene product by inhibiting the degradation of the gene or gene product. For example, in one embodiment, the modulator decreases the ubiquitination, proteosomal degradation, or proteolysis of one or more poly-D/E protein. In one embodiment, the modulator increases the stability or half-life of a gene product, for example, one or more poly-D/E protein.

Modulation of a gene, or gene product, can be assessed using a wide variety of methods, including those disclosed herein, as well as methods known in the art or to be developed in the future. That is, the routineer would appreciate, based upon the disclosure provided herein, that modulating the level or activity of a gene, or gene product, can be readily assessed using methods that assess the level of a nucleic acid encoding a gene product (e.g., mRNA), the level of polypeptide gene product present in a biological sample, the activity of polypeptide gene product present in a biological sample, or combinations thereof.

The modulator compositions and methods of the invention that modulate the level or activity of a gene, or gene product, include, but should not be construed as being limited to, a chemical compound, a protein, a peptide, a peptidomemetic, an antibody, a ribozyme, a small molecule chemical compound, a nucleic acid, a vector, an antisense nucleic acid molecule (e.g., siRNA, miRNA, etc.), or combinations thereof. One of skill in the art would readily appreciate, based on the disclosure provided herein, that a modulator composition encompasses a chemical compound that modulates the level or activity of a gene, or gene product. Additionally, a modulator composition encompasses a chemically modified compound, and derivatives, as is well known to one of skill in the chemical arts.

In one embodiment, the modulator composition of the present invention is an agonist, which increases the expression, activity, or biological function of a gene or gene product. For example, in certain embodiments, the modulator of the present invention is an agonist of one or more poly-D/E protein.

Further, one of skill in the art would, when equipped with this disclosure and the methods exemplified herein, appreciate that modulators include such modulators as discovered in the future, as can be identified by well-known criteria in the art of pharmacology, such as the physiological results of modulation of the genes, and gene products, as described in detail herein and/or as known in the art. Therefore, the present invention is not limited in any way to any particular modulator composition as exemplified or disclosed herein; rather, the invention encompasses those modulator compositions that would be understood by the routineer to be useful as are known in the art and as are discovered in the future.

Further methods of identifying and producing modulator compositions are well known to those of ordinary skill in the art. Alternatively, a modulator can be synthesized chemically. Further, the routineer would appreciate, based upon the teachings provided herein, that a modulator composition can be obtained from a recombinant organism. Compositions and methods for chemically synthesizing modulators and for obtaining them from natural sources are well known in the art and are described in the art.

One of skill in the art will appreciate that a modulator can be administered as a small molecule chemical, a polypeptide, a peptide, an antibody, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a peptide or a nucleic acid encoding a peptide that is modulator of a gene, or gene product. For example, the invention includes a peptide or a nucleic acid encoding a peptide that comprises one or more poly-D/E protein. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

### Peptides

In one embodiment, the composition of the present invention comprises one or more peptides. In one embodiment, a peptide of the composition comprises an amino acid sequence of one or more poly-D/E protein. In one embodiment, said poly-D/E protein comprises at least 15, at least 20, at least 25, at least 30, at least 35, at least 40, or at least 45 aspartic acid (D) or glutamic acid (E) residues within any given continuous stretch of at least 50, at least 55, at least 60, at least 65, at least 70, or at least 75 amino acids. In one embodiment, said poly-D/E protein comprises at least 35 aspartic acid (D) or glutamic acid (E) residues within any given continuous stretch of at least 50 amino acids.

In one embodiment, the poly-D/E protein comprises one or more selected from the group consisting of: Death domain-associated protein 6 (DAXX, alternatively ETS1-associated protein 1, EAP1); Acidic leucine-rich nuclear phosphoprotein 32 family member A (ANP32A or AN32A, alternatively Acidic nuclear phosphoprotein pp32, pp32, or Leucine-rich acidic nuclear protein, LANP); Protein SET (SET, alternatively HLA-DR-associated protein II or Inhibitor of granzyme A-activated DNase, IGAAD); E3 ubiquitin-protein ligase HUWE1 (HUWE1, alternatively ARF-binding protein 1, ARF-BP1); Transcription termination factor 4, mitochondrial (MTEF4); Myelin transcription factor 1 (MYT1 or MyTI); Sodium/potassium/calcium exchanger 1 (NCKX1, alternatively Retinal rod Na-C+K exchanger); Myelin transcription factor 1-like protein (MYT1L or MyT1-L); Glutamate-rich protein 6 (ERIP6, alternatively Protein FAM194A); Protein FAM9A (FAM9A); Eukaryotic translation initiation factor 5B (IF2P or eIF-5B, alternatively Translation initiation factor IF-2); Armadillo-like helical domain-containing protein 4 (ARMD4); Protein phosphatase 1G (PPM1G, alternatively Protein phosphatase magnesium-dependent 1 gamma); Ran GTPase-activating protein 1 (RAGP1 or RanGAP1); Nucleolin (NUCL or NCL, alternatively Protein C23); Nardilysin (NRDC, alternatively N-arginine dibasic convertase); Zinc finger homeobox protein 3 (ZFHX3 or ZFH-3, alternatively AT motif-binding factor 1); Zinc finger and BTB domain-containing protein 7C (ZBT7C, alternatively Affected by papillomavirus DNA integration in ME180 cells protein 1, APM-1); Zinc finger E-box-binding homeobox 1 (ZEB1, alternatively NIL-2-A zinc finger protein or Transcription factor 8, TCF-8); YTH domain-containing protein 1 (YTDC1, alternatively Splicing factor YT521, YT521B); Zinc finger and BTB domain-containing protein 47 (ZBT47); Tau-tubulin kinase 1 (TTBK1, alternatively Brain-derived tau kinase); Histone acetyltransferase KAT6B (KAT6B, alternatively Histone acetyltransferase MOZ2 or MOZ, YBF2/SAS3, SAS2 and TIP60 protein 4 (MYST-4)); Proline-glutamic acid- and leucine-rich protein 1 (PELP1, alternatively Transcription factor HMX3); Parathymosin (PTMS); Tripartite motif-containing protein 26 (TRI26, alternatively Acid finger protein, AFP, RING finger protein 95, or Zinc finger protein 173); Ryanodine 1 (RYR1 or RYR-1); Protein SETSIP (SETLP, alternatively SET pseudogene protein 18, SETSIP or SETP18); Claspin (CLSPN); Calreticulin (CALR, alternatively Calregulin or Endoplasmic reticulum resident protein 60, ERp60, or CRP55, HACBP, or grp60); Nucleosome-remodeling factor subunit BPTF (BPTF, alternatively Bromodomain and PHD finger-containing transcription factor or Fetal Alzheimer antigen); Bromodomain adjacent to zinc finger domain protein 2B (BAZ2B, alternatively hWALp4); ATPase family AAA domain-containing protein 2 (ATAD2, alternatively AAA nuclear coregulator cancer-associated protein, ANCCA); Cilia- and flagella-associated protein 65 (CFA65, alternatively Coiled-coil domain-containing protein 108); Major centromere autoantigen B (CENPB or CENP-B, alternatively Centromere protein B); Zinc finger protein castor homolog 1 (CASZ1, alternatively Castor-related protein or Zinc finger protein 693); Coiled-coil domain-containing glutamate-rich protein 1 (CCER1); DDB1- and CUL-4 associated factor 8-like protein 2 (DC8L2, alternatively WD repeat-containing protein 42C); DDB1- and CUL4-associatedfactor 1 (DCAF1, alternatively HIV-1 Vpr binding protein, VprBP); Acidic leucine-rich nuclear phosphoprotein 32 family member B (AN32B, alternatively Putative HLA-DR-associated protein I-2, PHAPI2); AT-rich interactive domain-containing protein 4B (ARI4B, alternatively ARID domain-containing 4B); Acidic leucine-rich nuclear phosphoprotein 32 family member E (AN32E, alternatively LANP-like protein (LANP-L)); Nucleolar transcription factor 1 (Alternatively Upstream-binding factor 1, UBF1); Histone-lysine N-methyltransferase SETD1B (SETD1B or SET1B, alternatively Lysine N-methyltransferase 2G, previously A0A0A0MQV9); and Protein virilizer homolog (VIR or VIRMA). In one embodiment, said poly-D/E protein comprises a human poly-D/E protein.

In one embodiment, the poly-D/E protein comprises a protein comprising an one or more amino acid sequences selected from the group consisting of SEQ ID NOS 1-45. SEQ ID NOs, and their corresponding UniProt identifiers, are shown in Table 1 below.

TABLE 1
SEQ ID NOs and UniProt identifiers for poly-D/E proteins

| SEQ ID NO: | UniProt ID |
|---|---|
| 1 | sp\|Q7Z6Z7\|HUWE1_HUMAN |
| 2 | sp\|Q7Z6M4\|MTEF4_HUMAN |
| 3 | sp\|Q01538\|MYT1_HUMAN |
| 4 | sp\|O60721\|NCKX1_HUMAN |
| 5 | sp\|Q9UL68\|MYT1L_HUMAN |
| 6 | sp\|Q7L0X2\|ERIP6_HUMAN |
| 7 | sp\|Q8IZU1\|FAM9A_HUMAN |
| 8 | sp\|O60841\|IF2P_HUMAN |
| 9 | sp\|Q86TY3\|ARMD4_HUMAN |
| 10 | sp\|O15355\|PPM1G_HUMAN |
| 11 | sp\|P46060\|RAGP1_HUMAN |
| 12 | sp\|P19338\|NUCL_HUMAN |
| 13 | sp\|O43847\|NRDC_HUMAN |
| 14 | sp\|Q15911\|ZFHX3_HUMAN |
| 15 | sp\|A1YPR0\|ZBT7C_HUMAN |
| 16 | sp\|P37275\|ZEB1_HUMAN |
| 17 | sp\|Q96MU7\|YTDC1_HUMAN |
| 18 | sp\|Q9UFB7\|ZBT47_HUMAN |
| 19 | sp\|Q5TCY1\|TTBK1_HUMAN |
| 20 | sp\|Q8WYB5\|KAT6B_HUMAN |
| 21 | sp\|Q8IZL8\|PELP1_HUMAN |
| 22 | sp\|P20962\|PTMS_HUMAN |
| 23 | sp\|Q12899\|TRI26_HUMAN |
| 24 | sp\|P21817\|RYR1_HUMAN |
| 25 | sp\|P0DME0\|SETLP_HUMAN |
| 26 | sp\|Q01105\|SET_HUMAN |
| 27 | sp\|Q9HAW4\|CLSPN_HUMAN |
| 28 | sp\|P27797\|CALR_HUMAN |
| 29 | sp\|Q12830\|BPTF_HUMAN |
| 30 | sp\|Q9UIF8\|BAZ2B_HUMAN |
| 31 | sp\|Q6PL18\|ATAD2_HUMAN |
| 32 | sp\|Q6ZU64\|CFA65_HUMAN |
| 33 | sp\|P07199\|CENPB_HUMAN |
| 34 | sp\|Q86V15\|CASZ1_HUMAN |
| 35 | sp\|Q8TC90\|CCER1_HUMAN |
| 36 | sp\|P0C7V8\|DC8L2_HUMAN |
| 37 | sp\|Q9UER7\|DAXX_HUMAN |
| 38 | sp\|Q9Y4B6\|DCAF1_HUMAN |
| 39 | sp\|Q92688\|AN32B_HUMAN |
| 40 | sp\|Q4LE39\|ARI4B_HUMAN |
| 41 | sp\|P39687\|AN32A_HUMAN |
| 42 | sp\|Q9BTT0\|AN32E_HUMAN |
| 43 | sp\|P17480\|UBF1_HUMAN |
| 44 | sp\|Q69YN4\|VIR_HUMAN |
| 45 | tr\|A0A0A0MQV9\|A0A0A0MQV9_HUMAN |

In one embodiment, the poly-D/E protein comprises one or more protein selected from Table 2 as shown below. UniProt identifiers and their corresponding protein names, are shown in Table 2 below. Further shown are the parameters of varying stringency identifying said proteins as poly-D/E proteins. For example, a “Y” in column 3 indicates at least 30 D or E residues within a continuous stretch of 50 amino acid residues.

TABLE 2
poly-D/E proteins as identified by varying parameters.

| | Protein | (D + E) in the AA window | | | |
|---|---|---|---|---|---|
| UniProt ID | Name | 30 in 50 | 35 in 50 | 40 in 70 | 45 in 100 |
| sp\|Q96KQ7\|EHMT2_HUMAN | EHMT2 | Y | | Y | Y |
| sp\|Q4G1C9\|GRPL2_HUMAN | GRPL2 | Y | | | |
| sp\|Q9H501\|ESF1_HUMAN | ESF1 | Y | | | Y |
| sp\|P09429\|HMGB1_HUMAN | HMGB1 | Y | | | |
| sp\|O43719\|HTSF1_HUMAN | HTSF1 | Y | | Y | Y |
| sp\|Q9Y4W2\|LAS1L_HUMAN | LAS1L | Y | | | |
| sp\|O75037\|KI21B_HUMAN | KI21B | Y | | | |
| sp\|Q5T7N2\|LITD1_HUMAN | LITD1 | Y | | | Y |
| sp\|Q13562\|NDF1_HUMAN | NDF1 | Y | | | |
| sp\|P07196\|NFL_HUMAN | NFL | Y | | Y | Y |
| sp\|Q9Y2I1\|NISCH_HUMAN | NISCH | Y | | | |
| sp\|Q9UK99\|FBX3_HUMAN | FBX3 | Y | | | |
| sp\|Q969H0\|FBXW7_HUMAN | FBXW7 | Y | | | |
| sp\|Q8IX15\|HOMEZ_HUMAN | HOMEZ | Y | | | |
| sp\|P78415\|IRX3_HUMAN | IRX3 | Y | | | Y |
| sp\|O95373\|IPO7_HUMAN | IPO7 | Y | | | |
| sp\|Q9UN42\|AT1B4_HUMAN | AT1B4 | Y | | | |
| sp\|P05067\|A4_HUMAN | A4 | Y | | Y | Y |
| sp\|Q13029\|PRDM2_HUMAN | PRDM2 | Y | | Y | Y |
| sp\|Q9NW13\|RBM28_HUMAN | RBM28 | Y | | | Y |
| sp\|Q9HAU5\|RENT2_HUMAN | RENT2 | Y | | | Y |
| sp\|Q9BT43\|RPC7L_HUMAN | RPC7L | Y | | Y | |
| sp\|Q15061\|WDR43_HUMAN | WDR43 | Y | | Y | Y |
| sp\|Q5BKZ1\|ZN326_HUMAN | ZN326 | Y | | | Y |
| sp\|Q969S3\|ZN622_HUMAN | ZN622 | Y | | | |
| sp\|P06454\|PTMA_HUMAN | PTMA | Y | | Y | Y |
| sp\|Q8WV44\|TRI41_HUMAN | TRI41 | Y | | | Y |
| sp\|Q96DX7\|TRI44_HUMAN | TRI44 | Y | | Y | Y |
| sp\|O00267\|SPT5H_HUMAN | SPT5H | Y | | Y | Y |
| sp\|Q5HYW3\|RTL5_HUMAN | RTL5 | Y | | | Y |
| sp\|P52655\|TF2AA_HUMAN | TF2AA | Y | | | |
| sp\|Q5H9L4\|TAF7L_HUMAN | TAF7L | Y | | | |
| sp\|Q9H1E5\|TMX4_HUMAN | TMX4 | Y | | | |
| sp\|Q7KZ85\|SPT6H_HUMAN | SPT6H | Y | | | |
| sp\|Q9ULL8\|SHRM4_HUMAN | SHRM4 | Y | | | |
| sp\|P23327\|SRCH_HUMAN | SRCH | Y | | Y | Y |
| sp\|Q2VWA4\|SKOR2_HUMAN | SKOR2 | Y | | | |
| sp\|P45379\|TNNT2_HUMAN | TNNT2 | Y | | Y | Y |
| sp\|Q9BQ16\|TICN3_HUMAN | TICN3 | Y | | | |
| sp\|P12270\|TPR_HUMAN | TPR | Y | | Y | Y |
| sp\|Q8IUR6\|CRERF_HUMAN | CRERF | Y | | Y | Y |
| sp\|Q5VXU3\|CHIC1_HUMAN | CHIC1 | Y | | | |
| sp\|Q8IX12\|CCAR1_HUMAN | CCAR1 | Y | | | Y |
| sp\|Q9UQ88\|CD11A_HUMAN | CD11A | Y | | Y | Y |
| sp\|P21127\|CD11B_HUMAN | CD11B | Y | | Y | Y |
| sp\|Q9P1Z9\|CC180_HUMAN | CC180 | Y | | | |
| sp\|O14958\|CASQ2_HUMAN | CASQ2 | Y | | | |
| sp\|Q99856\|ARISA_HUMAN | ARI3A | Y | | | |
| sp\|Q9HCE9\|ANO8_HUMAN | ANO8 | Y | | | |
| sp\|Q5JTC6\|AMER1_HUMAN | AMER1 | Y | | | |
| sp\|Q06481\|APLP2_HUMAN | APLP2 | Y | | Y | Y |
| sp\|P60006\|APC15_HUMAN | APC15 | Y | | | |
| sp\|O43423\|AN32C_HUMAN | AN32C | Y | | Y | Y |
| sp\|Q6ZQQ6\|WDR87_HUMAN | WDR87 | Y | | Y | Y |
| sp\|P82970\|HMGN5_HUMAN | HMGN5 | | | | Y |
| sp\|O00566\|MPP10_HUMAN | MPP10 | | | | Y |
| sp\|Q7RTP6\|MICA3_HUMAN | MICA3 | | | | Y |
| sp\|Q9NU22\|MDN1_HUMAN | MDN1 | | | | Y |
| sp\|P46821\|MAP1B_HUMAN | MAP1B | | | | Y |
| sp\|Q9ULW6\|NP1L2_HUMAN | NP1L2 | | | | Y |
| sp\|P07197\|NFM_HUMAN | NFM | | | | Y |
| sp\|Q5W0A0\|ERI6B_HUMAN | ERI6B | | | | Y |
| sp\|Q86X53\|ERIC1_HUMAN | ERIC1 | | | | Y |
| sp\|Q96QF7\|ACRC_HUMAN | ACRC | | | | Y |
| sp\|O95602\|RPA1_HUMAN | RPA1 | | | | Y |
| sp\|A6NFI3\|ZN316_HUMAN | ZN316 | | | | Y |
| sp\|Q5T200\|ZC3HD_HUMAN | ZC3HD | | | | Y |
| sp\|Q9H2G4\|TSYL2_HUMAN | TSYL2 | | | | Y |
| sp\|Q8N7H5\|PAF1_HUMAN | PAF1 | | | | Y |
| sp\|Q96A61\|TRI52_HUMAN | TRI52 | | | | Y |
| sp\|Q9UPS6\|SET1B_HUMAN | SET1B | | | | Y |
| sp\|Q9H1E5\|TMX4_HUMAN | TMX4 | | | | Y |
| sp\|Q9Y5B9\|SP16H_HUMAN | SP16H | | | | Y |
| sp\|Q7KZ85\|SPT6H_HUMAN | SPT6H | | | | Y |
| sp\|Q6ZRS2\|SRCAP_HUMAN | SRCAP | | | | Y |
| sp\|Q5MJ10\|SPXN2_HUMAN | SPXN2 | | | | Y |
| sp\|Q92794\|KAT6A_HUMAN | KAT6A | | | | Y |
| sp\|P49756\|RBM25_HUMAN | RBM25 | | | | Y |
| sp\|Q14028\|CNGB1_HUMAN | CNGB1 | | | | Y |
| sp\|Q14692\|BMS1_HUMAN | BMS1 | | | | Y |
| sp\|P51861\|CDR1_HUMAN | CDR1 | | | | Y |
| sp\|Q96A33\|CCD47_HUMAN | CCD47 | | | | Y |
| tr\|G3V1R5\|G3V1R5_HUMAN | N/A | Y | Y | Y | Y |
| tr\|B7Z4S5\|B7Z4S5_HUMAN | N/A | Y | | | |
| tr\|A0A0G2JIR1\|A0A0G2JIR1_HUMAN | N/A | Y | | Y | Y |
| tr\|A0A2R8YF72\|A0A2R8YF72_HUMAN | N/A | Y | Y | Y | Y |
| tr\|Q49AF1\|Q49AF1_HUMAN | N/A | Y | | | |
| tr\|MOR2M5\|MOR2M5_HUMAN | N/A | Y | | Y | Y |
| tr\|B4DFP7\|B4DFP7_HUMAN | N/A | Y | Y | Y | Y |
| tr\|Q5QPR3\|Q5QPR3_HUMAN | N/A | Y | | Y | Y |
| tr\|A0A0G2JRN8\|A0A0G2JRN8_HUMAN | N/A | Y | | | |
| tr\|A0A0A0MRN4\|A0A0A0MRN4_HUMAN | N/A | Y | | | Y |
| tr\|B7ZKW0\|B7ZKW0_HUMAN | N/A | Y | | | |
| tr\|B1AKJ5\|B1AKJ5_HUMAN | N/A | Y | Y | Y | Y |
| tr\|Q5TB25\|Q5TB25_HUMAN | N/A | Y | Y | Y | Y |
| tr\|C9JFV4\|C9JFV4_HUMAN | N/A | Y | Y | Y | Y |
| tr\|S4R3N3\|S4R3N3_HUMAN | N/A | Y | | | |
| tr\|A0A0C4DFV9\|A0A0C4DFV9_HUMAN | N/A | Y | Y | Y | Y |
| tr\|A0A0D9SER5\|A0A0D9SER5_HUMAN | N/A | Y | | Y | Y |
| tr\|J3QR21\|J3QR21_HUMAN | N/A | Y | | Y | Y |
| tr\|A0A087X027\|A0A087X027_HUMAN | N/A | Y | Y | Y | Y |
| tr\|E7EPW4\|E7EPW4_HUMAN | N/A | Y | | | |
| tr\|A0A0A0MRJ5\|A0A0A0MRJ5_HUMAN | N/A | Y | | | |
| tr\|F5GXF5\|F5GXF5_HUMAN | N/A | Y | Y | Y | Y |
| tr\|C9JVV3\|C9JVV3_HUMAN | N/A | Y | Y | | |
| tr\|E7ESW6\|E7ESW6_HUMAN | N/A | Y | | Y | Y |
| tr\|C9JM61\|C9JM61_HUMAN | N/A | Y | | | |
| tr\|F5H6E4\|F5H6E4_HUMAN | N/A | Y | | | |
| tr\|A0A0A0MR81\|A0A0A0MR81_HUMAN | N/A | Y | | | |
| tr\|J3KS35\|J3KS35_HUMAN | N/A | Y | | Y | Y |
| tr\|F5H127\|F5H127_HUMAN | N/A | Y | Y | Y | Y |
| tr\|H7C5G8\|H7C5G8_HUMAN | N/A | Y | | | |
| tr\|A0A087WX71\|A0A087WX71_HUMAN | N/A | Y | | | Y |
| tr\|M0QZ43\|M0QZ43_HUMAN | N/A | Y | | Y | Y |
| tr\|E7ETD6\|E7ETD6_HUMAN | N/A | Y | | | |
| tr\|A0A2R8Y7Q1\|A0A2R8Y7Q1_HUMAN | N/A | Y | | | |
| tr\|A0A0D9SEI3\|A0A0D9SEI3_HUMAN | N/A | Y | | Y | Y |
| tr\|B8ZZW7\|B8ZZW7_HUMAN | N/A | Y | | Y | Y |
| tr\|H7C2L2\|H7C2L2_HUMAN | N/A | Y | | | |
| tr\|S4R3U4\|S4R3U4_HUMAN | N/A | Y | | | |
| tr\|E7EV54\|E7EV54_HUMAN | N/A | Y | Y | Y | Y |
| tr\|Q5QPR4\|Q5QPR4_HUMAN | N/A | Y | | Y | Y |
| tr\|A0A0G2JRR0\|A0A0G2JRR0_HUMAN | N/A | Y | | | |
| tr\|G5EA39\|G5EA39_HUMAN | N/A | Y | | | |
| tr\|C9JAA9\|C9JAA9_HUMAN | N/A | Y | | | Y |
| tr\|A0A0A0MTJ2\|A0A0A0MTJ2_HUMAN | N/A | Y | | | |
| tr\|A0A2U3U043\|A0A2U3U043_HUMAN | N/A | Y | | Y | Y |
| tr\|A0A0A0MRG2\|A0A0A0MRG2_HUMAN | N/A | Y | | Y | Y |
| tr\|A0A087WUT6\|A0A087WUT6_HUMAN | N/A | Y | Y | Y | Y |
| tr\|H0Y9T3\|H0Y9T3_HUMAN | N/A | Y | | | Y |
| tr\|H0YFJ7\|H0YFJ7_HUMAN | N/A | Y | | | |
| tr\|E7ESP2\|E7ESP2_HUMAN | N/A | Y | | Y | Y |
| tr\|E7ESG2\|E7ESG2_HUMAN | N/A | Y | Y | Y | Y |
| tr\|E9PG40\|E9PG40_HUMAN | N/A | Y | | Y | Y |
| tr\|E9PE19\|E9PE19_HUMAN | N/A | Y | | | |
| tr\|E9PI45\|E9PI45_HUMAN | N/A | Y | Y | Y | Y |
| tr\|A0A087X0E6\|A0A087X0E6_HUMAN | N/A | Y | | | |
| tr\|C9JDF8\|C9JDF8_HUMAN | N/A | Y | | Y | Y |
| tr\|A0A0G2JIS2\|A0A0G2JIS2_HUMAN | N/A | Y | | Y | Y |
| tr\|H7C0V9\|H7C0V9_HUMAN | N/A | Y | | Y | Y |
| tr\|J3QR07\|J3QR07_HUMAN | N/A | Y | Y | Y | Y |
| tr\|A0A0G2JK64\|A0A0G2JK64_HUMAN | N/A | Y | | Y | Y |
| tr\|A6NGX6\|A6NGX6_HUMAN | N/A | Y | | Y | |
| tr\|F5H7V1\|F5H7V1_HUMAN | N/A | Y | Y | Y | Y |
| tr\|F5H483\|F5H483_HUMAN | N/A | Y | Y | Y | Y |
| tr\|H7C2N1\|H7C2N1_HUMAN | N/A | Y | | Y | Y |
| tr\|A0A0C4DGG8\|A0A0C4DGG8_HUMAN | N/A | Y | | | Y |
| tr\|B8ZZQ6\|B8ZZQ6_HUMAN | N/A | Y | | Y | Y |
| tr\|F5H3R3\|F5H3R3_HUMAN | N/A | Y | | | |
| tr\|A0A0D9SEN2\|A0A0D9SEN2_HUMAN | N/A | Y | | Y | Y |
| tr\|B8ZZA1\|B8ZZA1_HUMAN | N/A | Y | | Y | Y |
| tr\|E7EPN8\|E7EPN8_HUMAN | N/A | Y | | Y | Y |
| tr\|A2ABF9\|A2ABF9_HUMAN | N/A | Y | | Y | Y |
| tr\|G3V1E0\|G3V1E0_HUMAN | N/A | Y | | | |
| tr\|A2ABF8\|A2ABF8_HUMAN | N/A | Y | | Y | Y |
| tr\|J3QLA3\|J3QLA3_HUMAN | N/A | | | | Y |
| tr\|Q5JSK9\|Q5JSK9_HUMAN | N/A | | | | Y |
| tr\|A0A0C4DFX4\|A0A0C4DFX4_HUMAN | N/A | | | | Y |
| tr\|U3KQ48\|U3KQ48_HUMAN | N/A | | | | Y |
| tr\|A0A087X0E3\|A0A087X0E3_HUMAN | N/A | | | | Y |
| tr\|A0A0A0MS59\|A0A0A0MS59_HUMAN | N/A | | | | Y |
| tr\|E7EMV2\|E7EMV2_HUMAN | N/A | | | | Y |
| tr\|E7ESP9\|E7ESP9_HUMAN | N/A | | | | Y |
| tr\|H0YB25\|H0YB25_HUMAN | N/A | | | | Y |
| tr\|E5RHA3\|E5RHA3_HUMAN | N/A | | | | Y |

The invention should also be construed to include any form of a poly-D/E protein having substantial homology to the poly-D/E proteins disclosed herein, wherein said form maintains its activity as a molecular chaperone, disaggregase, unfoldase, or combination thereof. In some embodiments, the poly-D/E protein comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% homologous to a sequence as outlined in Table 1 above.

The invention should also be construed to include any form of a poly-D/E protein having substantial identity to the poly-D/E proteins disclosed herein, wherein said form maintains its activity as a molecular chaperone, disaggregase, unfoldase, or combination thereof. In some embodiments, the poly-D/E protein comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to a sequence as outlined in Table 1 above.

The invention should also be construed to include any form of a poly-D/E protein comprising a fragment of a poly-D/E protein disclosed herein, wherein said fragment maintains its activity as a molecular chaperone, disaggregase, unfoldase, or combination thereof. In some embodiments, the poly-D/E protein comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of a sequence as outlined in Table 1 above.

In one embodiment, the composition comprises a combination of the peptides described herein. In one embodiment, the composition comprises at least two poly-D/E proteins as described herein. In one embodiment, the composition comprises a fusion peptide comprising at least two poly-D/E proteins as described herein. In one embodiment, the composition comprises 1) one or more poly-D/E protein and 2) one or more tripartite motif (TRIM) protein. In one embodiment, the composition comprises a fusion peptide comprising 1) at least one poly-D/E protein and 2) at least one TRIM protein.

In some embodiments, the TRIM protein comprises one or more selected from the group consisting of: TRIM3, TRIM4, TRIM5, TRIM6, TRIM7, TRIM9, TRIM11, TRIM13, TRIM14, TRIM15, TRIM16, TRIM17, TRIM19 (also referred to as “PML”), TRIM20, TRIM21, TRIM24, TRIM25, TRIM27, TRIM28, TRIM29, TRIM32, TRIM34, TRIM39, TRIM43, TRIM44, TRIM45, TRIM46, TRIM49, TRIM50, TRIM52, TRIM58, TRIM59, TRIM65, TRIM67, TRIM69, TRIM70, TRIM74 and TRIM75; and TRIM30. In one embodiment, said one or more TRIM protein is a human TRIM protein. In one embodiment, said one or more TRIM protein is a mouse TRIM protein. TRIM proteins and their use for preventing or treating protein misfolding and aggregation are disclosed in International (PCT) Publication Number: WO 2016/196328 A1, incorporated by reference herein in its entirety.

In certain embodiments, the peptide comprises a targeting domain, which targets the peptide to a desired location. For example, in certain embodiments, the targeting domain binds to a targeted cell, protein, or protein aggregate, thereby delivering the therapeutic peptide to a desired location. For example, in one embodiment, the targeting domain is directed to bind to a protein or protein aggregate associated with a disease or disorder, including but not limited to the proteins and protein aggregates of amyloid-beta, alpha-synuclein, tau, prions, SOD1, TDP-43, FUS, p53, p53 mutants, and proteins associated with polyglutamine repeats, such as huntingtin and ataxins.

In certain embodiments, the targeting domain comprises a peptide, nucleic acid, small molecule, or the like, which has the ability to bind to the targeted cell, protein, or protein aggregate. For example, in one embodiment, the targeting domain comprises an antibody or antibody fragment which binds to a targeted cell, protein, or protein aggregate.

In one embodiment, the peptide comprises a secretory signal peptide. For example, in one embodiment, the peptide is a fusion peptide comprising a secretory signal peptide fused (either directly or via a linker domain) to a poly-D/E protein, as described herein. For example, in one embodiment, the peptide comprises a fusion peptide comprising a secretory signal peptide fused to a poly-D/E protein selected from: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR. In certain embodiments, the secretory signal peptide targets the fusion peptide for translocation across the endoplasmic reticulum membrane and into the secretory pathway. In one embodiment, the fusion peptide comprises a proteolytic site between the secretory signal peptide and the rest of the peptide.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly (sialic acid), dextran, poly (N-(2-hydroxypropyl) methacrylamide) (PHPMA), poly (N-vinylpyrrolidone) (PVP), poly (vinyl alcohol) (PVA), poly (ethylene glycol-co-propylene glycol), poly (N-acryloyl morpholine (PAcM), and poly (ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12:429-456) excellent solubility in aqueous solution (Powell, supra), low immunogenicity and antigenicity (Dreborg et al., 1990, Crit. Rev. Ther. Drug Carrier Syst. 6:315-365). PEG-conjugated or “PEGylated” protein therapeutics, containing single or multiple chains of polyethylene glycol on the protein, have been described in the scientific literature (Clark et al., 1996, J. Biol. Chem. 271:21969-21977; Hershfield, 1997, Biochemistry and immunology of poly (ethylene glycol)-modified adenosine deaminase (PEG-ADA). In J. M. Harris and S. Zalipsky (Eds) Poly (ethylene glycol): Chemistry and Biological Applications. American Chemical Society, Washington, D.C., p 145-154; Olson et al., 1997, Preparation and characterization of poly (ethylene glycol) ylated human growth hormone antagonist. In J. M. Harris and S. Zalipsky (Eds) Poly (ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

The peptides of the invention may be prepared by standard chemical or biological means of peptide synthesis. Biological methods include, without limitation, expression of a nucleic acid encoding a peptide in a host cell or in an in vitro translation system.

Included in the invention are nucleic acid sequences that encode the peptide of the invention. In one embodiment, the invention includes nucleic acid sequences encoding the amino acid sequence of one or more poly-D/E protein. Accordingly, subclones of a nucleic acid sequence encoding a peptide of the invention can be produced using conventional molecular genetic manipulation for subcloning gene fragments, such as described by Sambrook et al., Molecular Cloning: A Laboratory Manual, Cold Springs Laboratory, Cold Springs Harbor, New York (2012), and Ausubel et al. (ed.), Current Protocols in Molecular Biology, John Wiley & Sons (New York, NY) (1999 and preceding editions), each of which is hereby incorporated by reference in its entirety. The subclones then are expressed in vitro or in vivo in bacterial cells to yield a smaller protein or polypeptide that can be tested for a particular activity.

Combined with certain formulations, such peptides can be effective intracellular agents. However, in order to increase the efficacy of such peptides, the one or more peptides of the invention can be provided a fusion peptide along with a second peptide which promotes “transcytosis”, e.g., uptake of the peptide by cells. For example, in one embodiment, the peptide may comprise a cell-penetrating domain, for example a cell-penetrating peptide (CPP) to allow for the peptide to enter a cell. In one embodiment, the CPP is derived from HIV Tat.

To illustrate, the one or more peptides of the present invention can be provided as part of a fusion polypeptide with all or a fragment of the N-terminal domain of the HIV protein Tat, e.g., residues 1-72 of Tat or a smaller fragment thereof which can promote transcytosis. In one embodiment, the peptide comprises the protein transduction domain of HIV Tat. In other embodiments, the one or more peptides can be provided a fusion polypeptide with all or a portion of the antenopedia III protein. Other cell-penetrating domains that mediate uptake of the peptide are known in the art, and are equally applicable for use in a fusion peptide of the present invention.

### Nucleic Acids

In one embodiment, the composition of the invention comprises one or isolated nucleic acids. In one embodiment, the isolated nucleic acid encodes one or more poly-D/E protein. In one embodiment, the isolated nucleic acid encodes one or more poly-D/E protein comprising at least 15, at least 20, at least 25, at least 30, at least 35, at least 40, or at least 45 aspartic acid (D) or glutamic acid (E) residues within any given continuous stretch of at least 50, at least 55, at least 60, at least 65, at least 70, or at least 75 amino acids. In one embodiment, the isolated nucleic acid encodes one or more poly-D/E protein comprises at least 35 aspartic acid (D) or glutamic acid (E) residues within any given continuous stretch of at least 50 amino acids.

In one embodiment, the isolated nucleic acid encodes one or more poly-D/E protein selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

In one embodiment, the isolated nucleic acid encodes one or more poly-D/E protein comprising one or more sequence selected from the group consisting of SEQ ID NOs 1-45. SEQ ID NOs, and their corresponding UniProt identifiers, are shown in Table 1 above. In one embodiment, the isolated nucleic acid encodes one or more poly-D/E protein comprising one or more protein selected from Table 2 as shown above. In certain embodiments, a peptide corresponding to one or more poly-D/E protein is expressed from the one or more nucleic acids in a cell in vivo or in vitro using known techniques.

The nucleotide sequence of the isolated nucleic acids include both the DNA sequence that is transcribed into RNA and the RNA sequence that is translated into a polypeptide. According to other embodiments, the nucleotide sequences are inferred from the amino acid sequence of the peptides of the invention. As is known in the art several alternative nucleotide sequences are possible due to redundant codons, while retaining the biological activity of the translated peptides.

The invention also encompasses any nucleic acid having substantial homology to a nucleotide sequence as disclosed herein, wherein said isolated nucleic acid encodes a poly-D/E protein that maintains its activity as a molecular chaperone, disaggregase, unfoldase, or combination thereof. In some embodiments, the isolated nucleic acid comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% homologous to a nucleotide sequence as outlined in Table 1 above.

The invention should also be construed to include any nucleic acid having substantial identity to a nucleotide sequence as disclosed herein, wherein said isolated nucleic acid encodes ap poly-D/E protein that maintains its activity as a molecular chaperone, disaggregase, unfoldase, or combination thereof. In some embodiments, the isolated nucleic acid comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to a nucleotide sequence as outlined in Table 1 above.

The invention should also be construed to include any fragment of a nucleic acid encoding a fragment of a poly-D/E protein disclosed herein, wherein said isolated nucleic acid encodes a fragment that maintains its activity as a molecular chaperone, disaggregase, unfoldase, or combination thereof. In some embodiments, the isolated nucleic acid comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of a nucleotide sequence as outlined in Table 1 above.

In one embodiment, the nucleic acid encodes a fusion peptide, such as a fusion peptide comprising a targeting domain and/or a secretory signal peptide fused to a poly-D/E protein, as described herein. In one embodiment, the nucleic acid encodes a peptide comprising a secretory signal peptide. For example, in one embodiment, the nucleic acid encodes a fusion peptide comprising a secretory signal peptide fused (either directly or via a linker domain) to a poly-D/E protein, as described herein. For example, in one embodiment, the nucleic acid encodes a fusion peptide comprising a secretory signal peptide fused to a poly-D/E protein selected from: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR. In certain embodiments, the secretory signal peptide targets the fusion peptide for translocation across the endoplasmic reticulum membrane and into the secretory pathway. In one embodiment, the fusion peptide comprises a proteolytic site between the secretory signal peptide and the rest of the peptide.

In one embodiment, the composition comprises a combination of the nucleic acid molecules described herein. For example, in certain embodiments, the composition comprises an isolated nucleic acid molecule encoding at least two poly-D/E proteins as disclosed herein. In one embodiment, the composition comprises at least two isolated nucleic acid molecules encoding at least two poly-D/E proteins. In one embodiment, the composition comprises 1) one or more nucleic acid encoding one or more poly-D/E protein and 2)) one or more nucleic acid encoding one or more TRIM protein. In one embodiment, the composition comprises a nucleic acid encoding 1) at least one poly-D/E protein and 2) at least one TRIM protein. In one embodiment, the composition comprises a nucleic acid encoding a fusion peptide comprising 1) at least one poly-D/E protein and 2) at least one TRIM protein.

In some embodiments, the TRIM protein encoded by the nucleic acid comprises one or more selected from the group consisting of: TRIM3, TRIM4, TRIM5, TRIM6, TRIM7, TRIM9, TRIM11, TRIM13, TRIM14, TRIM15, TRIM16, TRIM17, TRIM19 (also referred to as “PML”), TRIM20, TRIM21, TRIM24, TRIM25, TRIM27, TRIM28, TRIM29, TRIM32, TRIM34, TRIM39, TRIM43, TRIM44, TRIM45, TRIM46, TRIM49, TRIM50, TRIM52, TRIM58, TRIM59, TRIM65, TRIM67, TRIM69, TRIM70, TRIM74 and TRIM75; and TRIM30. In one embodiment, said one or more TRIM protein is a human TRIM protein. In one embodiment, said one or more TRIM protein is a mouse TRIM protein. Nucleic acids encoding TRIM proteins and their use for preventing or treating protein misfolding and aggregation are disclosed in International (PCT) Publication Number: WO 2016/196328 A1, incorporated by reference herein in its entirety.

Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

The desired nucleic acid encoding one or more poly-D/E protein can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In one embodiment, the encoding sequence is contained within an AAV vector. More than 30 naturally occurring serotypes of AAV are available. Many natural variants in the AAV capsid exist, allowing identification and use of an AAV with properties specifically suited for the tissue of interest. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Thus, expression of one or more poly-D/E protein can be achieved by delivering a recombinantly engineered AAV or artificial AAV that contains one or more encoding sequences. The use of AAVs is a common mode of exogenous delivery of DNA as it is relatively non-toxic, provides efficient gene transfer, and can be easily optimized for specific purposes. Exemplary AAV serotypes include, but is not limited to AAV1, AAV2, AAV3, AAV4, AAV5, AAV6, AAV7, AAV8 and AAV9.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of one or more poly-D/E protein include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAVrh8 (International Patent Publication No. WO2003/042397), among others.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In one embodiment, the promoter or enhancer specifically directs expression of the one or more poly-D/E protein in neural tissue. For example, in certain embodiments, the promoter or enhancer specifically directs expression of the one or more poly-D/E protein in a neuron, astrocyte, oligodendrocyte, Perkinje cell, pyramidal cell, or the like.

In one embodiment, the promoter or enhancer specifically directs expression of the one or more poly-D/E protein in cancerous tissue. For example, in certain embodiments, the promoter or enhancer specifically directs expression of the one or more poly-D/E protein in tissues including, but not limited to, those associated with epithelial cancer, cholangiocarcinoma, melanoma, colon cancer, rectal cancer, ovarian cancer, endometrial cancer, non-small cell lung cancer (NSCLC), glioblastoma, uterine cervical cancer, head and neck cancer, breast cancer, pancreatic cancer, bladder cancer.

In order to assess the expression of the desired polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

In one embodiment, the composition of the invention comprises RNA encoding one or more poly-D/E protein, as described herein. In one embodiment, the RNA encodes one or more poly-D/E protein selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

In one embodiment, the RNA encodes one or more poly-D/E protein comprising one or more sequence selected from the group consisting of SEQ ID NOs 1-45. SEQ ID NOs, and their corresponding UniProt identifiers, are shown in Table 1 above. In one embodiment, the isolated nucleic acid encodes one or more poly-D/E protein comprising one or more protein selected from Table 2 as shown above.

In one embodiment, the composition comprises in vitro transcribed (IVT) RNA encoding one or more components of the one or more poly-D/E protein. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is one or more poly-D/E protein.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

In one embodiment, the composition of the present invention comprises a modified nucleic acid encoding one or more one or more poly-D/E protein described herein. For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low immunogenicity, and enhanced translation. Nucleoside-modified mRNA useful in the present invention is further described in U.S. Pat. No. 8,278,036, which is incorporated by reference herein in its entirety.

### Modified Cell

The present invention includes a composition comprising a cell which comprises one or more poly-D/E protein, a nucleic acid encoding a one or more poly-D/E protein, or a combination thereof. In one embodiment, the cell is genetically modified to express a protein and/or nucleic acid of the invention. In certain embodiments, genetically modified cell is autologous to a subject being treated with the composition of the invention. Alternatively, the cells can be allogeneic, syngeneic, or xenogeneic with respect to the subject. In certain embodiment, the cell is able to secrete or release the expressed protein into extracellular space in order to deliver the peptide to one or more other cells.

The genetically modified cell may be modified in vivo or ex vivo, using techniques standard in the art. Genetic modification of the cell may be carried out using an expression vector or using a naked isolated nucleic acid construct.

In one embodiment, the cell is obtained and modified ex vivo, using an isolated nucleic acid encoding one or more proteins described herein. In one embodiment, the cell is obtained from a subject, genetically modified to express the protein and/or nucleic acid, and is re-administered to the subject. In certain embodiments, the cell is expanded ex vivo or in vitro to produce a population of cells, wherein at least a portion of the population is administered to a subject in need.

In one embodiment, the cell is genetically modified to stably express the protein. In another embodiment, the cell is genetically modified to transiently express the protein.

### Therapeutic Methods

The present invention also provides therapeutic methods for a disease or disorder associated with protein misfolding, protein aggregates, or a combination thereof.

In one embodiment, the present invention provides a method of administering a composition comprising a modulator of one or more poly-D/E protein to a subject. In one embodiment, the subject has a disease or disorder associated with protein misfolding or protein aggregates. In one embodiment, the subject has a disease or disorder associated with misfolded proteins and/or and protein aggregates of amyloid-beta, alpha-synuclein, tau, prions, SOD1, TDP-43, FUS, p53, p53 mutants, or proteins associated with polyglutamine repeats, such as huntingtin and ataxins.

In various embodiments, diseases and disorders treatable by the methods of the invention include, but are not limited to: polyQ disorders such as SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), synucleinopathies, dementia with Lewy bodies (DLB), multiple system atrophy (MSA), tauopathies, Frontotemporal lobar degeneration (FTLD), AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis.

In certain embodiments, the method comprises the treatment or prevention of cancer associated with p53 aggregates. In some embodiments, said cancer comprises a cancer associated with one or more p53 mutation. In some embodiments, said cancer comprises a cancer associated with a mutation at one or more amino acid residue position selected from the group consisting of: 175, 220, 245, 248, 249, 273, 280, and 282, relative to the sequence for human p53. In some embodiments said cancer comprises a cancer associated with one or more p53 mutation including, but not limited to: R175H, Y220C, G245D, G245S, R248L, R248Q, R248W, R249S, R273H, R273C, R273L, R280K, or R282W of human p53. In one embodiment, said cancer comprises a cancer associated with one or more p53 conformational mutation. In one embodiment, said cancer comprises a cancer associated with one or more p53 mutation including, but not limited to: R175X, G245X, R249X, R280X, or G245X, wherein X denotes any amino acid mutation. In one embodiment, said cancer comprises a cancer associated with one or more p53 conformational mutation including, but not limited to: R175H, G245S, R249S, R280K, or G245D.

In some embodiments, the method comprises the treatment or prevention of one or more cancer including, but not limited to, acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colocrectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer.

It will be appreciated by one of skill in the art, when armed with the present disclosure including the methods detailed herein, that the invention is not limited to treatment of a disease associated with protein misfolding or protein aggregates that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with protein misfolding or protein aggregates, in that a modulator composition, as discussed previously elsewhere herein, can be administered to a subject prior to the onset of the disease or disorder, thereby preventing the disease or disorder.

One of skill in the art, when armed with the disclosure herein, would appreciate that the prevention of a disease associated with protein misfolding or protein aggregates, encompasses administering to a subject a modulator as a preventative measure against the development of, or progression of a disease associated with protein misfolding or protein aggregates. As more fully discussed elsewhere herein, methods of modulating the level or activity of a gene, or gene product, encompass a wide plethora of techniques for modulating not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases associated with protein misfolding or protein aggregates, where modulating the level or activity of a gene, or gene product treats or prevents the disease. Various methods for assessing whether a disease is associated protein misfolding or protein aggregates are known in the art. Further, the invention encompasses treatment or prevention of such diseases discovered in the future.

In one aspect, the method comprises use of one or more poly-D/E protein to stabilize a misfolded protein. In certain aspects, stabilization of a functional misfolded protein via one or more poly-D/E protein described herein can treat or prevent a disease or disorder associated with the misfolded protein. For example, in one embodiment, stabilization of mutant cystic fibrosis transmembrane conductance regulator (CFTR), via one or more poly-D/E protein described herein, would allow mutant CFTR to function instead of being degraded. It is envisioned that using poly-D/E proteins to stabilize misfolded proteins can be used to treat cystic fibrosis and other diseases associated with degradation of partially functional proteins. Stabilization of proteins, via one or more poly-D/E protein described herein, can be used to treat any disease or disorder associated with degradation of functional mutant protein, including but not limited to cystic fibrosis and lysosomal storage diseases such as Gaucher's disease and Fabry's disease.

The invention encompasses administration of a modulator of a gene, or gene product. To practice the methods of the invention; the skilled artisan would understand, based on the disclosure provided herein, how to formulate and administer the appropriate modulator composition to a subject. The present invention is not limited to any particular method of administration or treatment regimen.

In one embodiment, the method comprises administering to the subject in need an effective amount of a composition that increases the expression or activity of one or more poly-D/E protein.

For example, in one embodiment, the method comprises administering to the subject in need an effective amount of a composition that increases the expression or activity of one or more of poly-D/E protein. In one embodiment, the method comprises administering to the subject in need 1) an effective amount of a composition that increases the expression or activity of one or more of poly-D/E protein and 2) an effective amount of a composition that increases the expression or activity of one or more of TRIM protein.

In one embodiment, the poly-D/E protein comprises one or more selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR. In one embodiment, said poly-D/E protein comprises a human poly-D/E protein.

In one embodiment, the poly-D/E protein comprises one or more sequence selected from the group consisting of SEQ ID NOs 1-45. SEQ ID NOs, and their corresponding UniProt identifiers, are shown in Table 1 above. In one embodiment, the poly-D/E protein comprises one or more protein selected from Table 2 as shown above. In some embodiments, the TRIM protein comprises one or more selected from the group consisting of: TRIM3, TRIM4, TRIM5, TRIM6, TRIM7, TRIM9, TRIM11, TRIM13, TRIM14, TRIM15, TRIM16, TRIM17, TRIM19 (also referred to as “PML”), TRIM20, TRIM21, TRIM24, TRIM25, TRIM27, TRIM28, TRIM29, TRIM32, TRIM34, TRIM39, TRIM43, TRIM44, TRIM45, TRIM46, TRIM49, TRIM50, TRIM52, TRIM58, TRIM59, TRIM65, TRIM67, TRIM69, TRIM70, TRIM74 and TRIM75; and TRIM30. In one embodiment, said one or more TRIM protein is a human TRIM protein. In one embodiment, said one or more TRIM protein is a mouse TRIM protein. Methods of using TRIM proteins, nucleic acids encoding TRIM proteins, and combinations thereof for preventing or treating protein misfolding and aggregation are disclosed in International (PCT) Publication Number: WO 2016/196328 A1, incorporated by reference herein in its entirety.

In one embodiment, the method comprises administering to the subject in need 1) an effective amount of a composition that increases the expression or activity of one or more of poly-D/E protein and 2) an effective amount of a composition that increases the expression or activity of one or more of TRIM protein.

In one embodiment, the method comprises increasing the expression or activity of the one or more poly-D/E protein in at least one neural cell of the subject. For example, in certain embodiments, the method comprises increasing the expression or activity of the one or more poly-D/E protein in a at least one neuron, glial cell, astrocyte, oligodendrocyte, Purkinje cell, pyramidal cell, or the like.

In one embodiment, the method comprises contacting the neural tissue of a subject with an effective amount of a composition that increases the expression or activity of one or more components of the one or more poly-D/E protein. For example, in certain embodiments, the method comprises contacting a neuron, glial cell, astrocyte, oligodendrocyte, Purkinje cell, pyramidal cell, or the like, of a subject with an effective amount of a composition that increases the expression or activity of one or more poly-D/E protein. In one embodiment, the neural cell is affected by protein misfolding, protein aggregates, or a combination thereof.

In one embodiment, the method comprises increasing the expression or activity of the one or more poly-D/E protein in at least one cancer cell of the subject. In some embodiments, the method comprises contacting one or more cancer cell with an effective amount of a composition that increases the expression or activity of one or more poly-D/E protein. In some embodiments, the cancer is associated with p53 aggregation.

One of skill in the art will appreciate that the modulators of the invention can be administered singly or in any combination. Further, the modulators of the invention can be administered singly or in any combination in a temporal sense, in that they may be administered concurrently, or before, and/or after each other. One of ordinary skill in the art will appreciate, based on the disclosure provided herein, that the modulator compositions of the invention can be used to prevent or to treat a disease or disorder associated with a misfolded protein or protein aggregate, and that a modulator composition can be used alone or in any combination with another modulator to effect a prophylactic or therapeutic result.

In various embodiments, any of the modulators of the invention described herein can be administered alone or in combination with other modulators of other molecules associated with a disease associated with protein misfolding or protein aggregates. In various embodiments, any of the modulators of the invention described herein can be administered alone or in combination with other therapeutic or preventative agents which may be used to treat or prevent a disease associated with protein misfolding or protein aggregates. Exemplary therapeutic agents which may be used in combination with the modulators of the present invention include, but is not limited to, anti-amyloid-β antibodies and anti-tau antibodies.

### Gene Therapy

Contacting cells in a subject with a nucleic acid composition that encodes a protein that increases the expression or activity of one or more poly-D/E can inhibit or delay the onset of one or more symptoms of a disease or disorder associated with protein misfolding or protein aggregates.

In one embodiment, the nucleic acid composition of the present invention encodes one or more peptides. For example, in one embodiment, a nucleic acid composition can encode a peptide that comprises an amino acid sequence of one or more poly-D/E proteins. In one embodiment, the nucleic acid composition encodes one or more poly-D/E protein selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

In one embodiment, the nucleic acid composition encodes one or more poly-D/E protein comprising one or more sequence selected from the group consisting of SEQ ID NOs 1-45. SEQ ID NOs, and their corresponding UniProt identifiers, are shown in Table 1 above. In one embodiment, the nucleic acid composition encodes one or more poly-D/E protein comprising one or more protein selected from Table 2 as shown above.

According to the present invention, a method is also provided of supplying protein to a cell which carries a normal, or a mutant gene, associated with diminished or insufficient activity of one or more poly-D/E protein. Supplying protein to a cell with a mutant gene should allow normal functioning of the recipient cells. The nucleic acid encoding a peptide may be introduced into the cell in a vector such that the nucleic acid remains extrachromosomal. In such a situation, the nucleic acid will be expressed by the cell from the extrachromosomal location. More preferred is the situation where the nucleic acid or a part thereof is introduced into the cell in such a way that it integrates into the cell's genome or recombines with the endogenous mutant gene present in the cell. Vectors for introduction of genes both for recombination, for integration, and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner.

As generally discussed above, a nucleic acid, where applicable, may be employed in gene therapy methods in order to increase the level or activity of the peptides of the invention even in those persons in which the wild type gene is expressed at a “normal” level, but the gene product is insufficiently functional.

“Gene therapy” includes both conventional gene therapy where a lasting effect is achieved by a single treatment, and the administration of gene therapeutic agents, which involves the one time or repeated administration of a therapeutically effective DNA or mRNA. Oligonucleotides can be modified to enhance their uptake, e.g., by substituting their negatively charged phosphodiester groups by uncharged groups. One or more poly-D/E protein of the present invention can be delivered using gene therapy methods, for example locally in neural cell or tissue or systemically (e.g., via vectors that selectively target specific tissue types, for example, tissue-specific adeno-associated viral vectors). In some embodiments, primary cells harvested from the individual can be transfected ex vivo with a nucleic acid encoding any of the peptides of the present invention, and then returned the transfected cells to the individual's body.

Gene therapy methods are well known in the art. See, e.g., WO96/07321 which discloses the use of gene therapy methods to generate intracellular antibodies. Gene therapy methods have also been successfully demonstrated in human patients. See, e.g., Baumgartner et al., Circulation 97:12, 1114-1123 (1998), Fatham, C.G. ‘A gene therapy approach to treatment of autoimmune diseases’, Immun. Res. 18:15-26 (2007); and U.S. Pat. No. 7,378,089, both incorporated herein by reference. See also Bainbridge JWB et al. “Effect of gene therapy on visual function in Leber's congenital Amaurosis”. N Engl J Med 358:2231-2239, 2008; and Maguire A M et al. “Safety and efficacy of gene transfer for Leber's Congenital Amaurosis”. N Engl J Med 358:2240-8, 2008.

There are two major approaches for introducing a nucleic acid encoding a peptide or protein (optionally contained in a vector) into a patients cells; in vivo and ex vivo. For in vivo delivery, in certain instances, the nucleic acid is injected directly into the patient, sometimes at the site where the protein is most required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. Commonly used vectors for ex vivo delivery of the gene are retroviral and lentiviral vectors.

Gene therapy would be carried out according to generally accepted methods, for example, as described by Friedman et al., 1991, Cell 66:799-806 or Culver, 1996, Bone Marrow Transplant 3: S6-9; Culver, 1996, Mol. Med. Today 2:234-236. In one embodiment, cells from a patient would be first analyzed by the diagnostic methods known in the art, to ascertain the expression or activity of one or more poly-D/E protein. A virus or plasmid vector, containing a copy of the gene or a functional equivalent thereof linked to expression control elements and capable of replicating inside the cells, is prepared. The vector may be capable of replicating inside the cells. Alternatively, the vector may be replication deficient and is replicated in helper cells for use in gene therapy. Suitable vectors are known, such as disclosed in U.S. Pat. No. 5,252,479 and PCT published application WO 93/07282 and U.S. Pat. Nos. 5,691,198; 5,747,469; 5,436,146 and 5,753,500. The vector is then injected into the patient. If the transfected gene is not permanently incorporated into the genome of each of the targeted cells, the treatment may have to be repeated periodically.

Gene transfer systems known in the art may be useful in the practice of the gene therapy methods of the present invention. These include viral and nonviral transfer methods. A number of viruses have been used as gene transfer vectors or as the basis for repairing gene transfer vectors, including papovaviruses (e.g., SV40, Madzak et al., 1992, J. Gen. Virol. 73:1533-1536), adenovirus (Berkner, 1992; Curr. Topics Microbiol. Immunol. 158:39-66), vaccinia virus (Moss, 1992, Current Opin. Biotechnol. 3:518-522; Moss, 1996, PNAS 93:11341-11348), adeno-associated virus (Russell and Hirata, 1998, Mol. Genetics 18:325-330), herpesviruses including HSV and EBV (Fink et al., 1996, Ann. Rev. Neurosci. 19:265-287), lentiviruses (Naldini et al., 1996, PNAS 93:11382-11388), Sindbis and Semliki Forest virus (Berglund et al., 1993, Biotechnol. 11:916-920), and retroviruses of avian (Petropoulos et al., 1992, J. Virol. 66:3391-3397), murine (Miller, 1992, Hum. Gene Ther. 3:619-624), and human origin (Shimada et al., 1991; Helseth et al., 1990; Page et al., 1990; Buchschacher and Panganiban, 1992, J. Virol. 66:2731-2739). Most human gene therapy protocols have been based on disabled murine retroviruses, although adenovirus and adeno-associated virus are also being used.

Nonviral gene transfer methods known in the art include chemical techniques such as calcium phosphate coprecipitation; mechanical techniques, for example microinjection; membrane fusion-mediated transfer via liposomes; and direct DNA uptake and receptor-mediated DNA transfer (Curiel et al., 1992, Am. J. Respir. Cell. Mol. Biol 6:247-252). Viral-mediated gene transfer can be combined with direct in vitro gene transfer using liposome delivery, allowing one to direct the viral vectors to the tumor cells and not into the surrounding non-dividing cells. Injection of producer cells would then provide a continuous source of vector particles. This technique has been approved for use in humans with inoperable brain tumors.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged. For other techniques for the delivery of adenovirus based vectors see U.S. Pat. Nos. 5,691,198; 5,747,469; 5,436,146 and 5,753,500.

Liposome/DNA complexes have been shown to be capable of mediating direct in vivo gene transfer. While in standard liposome preparations the gene transfer process is nonspecific, localized in vivo uptake and expression have been reported in tumor deposits, for example, following direct in situ administration.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes a protein, expression will produce the protein. If the polynucleotide encodes an antisense polynucleotide or a ribozyme, expression will produce the antisense polynucleotide or ribozyme. Thus in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

In certain embodiments, the method comprises the use of gene transfer techniques which target an isolated nucleic acid directly to neural tissue. Receptor-mediated gene transfer, for example, is accomplished by the conjugation of a nucleic acid molecule (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, co-infection with adenovirus can be included to disrupt endosome function.

### Pharmaceutical Compositions and Formulations

The invention also encompasses the use of pharmaceutical compositions of the invention or salts thereof to practice the methods of the invention. Such a pharmaceutical composition may consist of at least one modulator composition of the invention or a salt thereof in a form suitable for administration to a subject, or the pharmaceutical composition may comprise at least one modulator composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

In one embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intracerebral, epidural, intracerebroventricular, intraduodenal, intragastrical, intrathecal, subcutaneous, intramuscular, intradermal, intra-arterial, intravenous, intrabronchial, inhalation, and topical administration. In one embodiment, the composition can be administered to the cerebrospinal fluid of a subject.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Diagnostic Methods

The present invention provides a method to diagnose a subject having or at risk for developing a disease or disorder associated with protein misfolding or protein aggregates. For example, in one embodiment, the method comprises using the level of expression or activity of one or more poly-D/E protein as diagnostic markers. In one embodiment, the method comprises detecting the presence of a genetic mutation in a nucleic acid encoding one or more poly-D/E protein.

In one embodiment, the method is used to diagnose a subject as having a disease or disorder associated with protein misfolding or protein aggregates. In one embodiment, the method is used to diagnose a subject as being at risk for developing a disease or disorder associated with protein misfolding or protein aggregates.

In one embodiment, the method is used to evaluate the effectiveness of a therapy for a neurodegenerative disease or disorder associated with protein misfolding or protein aggregates. In one embodiment, the method is used to evaluate the effectiveness of a therapy for a cancer associated with protein misfolding or protein aggregates.

In one embodiment, the method comprises collecting a biological sample from a subject. Exemplary samples include, but are not limited to blood, urine, feces, sweat, bile, serum, plasma, tissue biopsy, and the like. For example, in one embodiment, the sample comprises at least one cell of neural tissue. In one embodiment, the sample comprises a neuron, astrocyte, oligodendrocyte, Perkinje cell, pyramidal cell, or the like. In one embodiment, the sample comprises any cell from a tissue at risk for developing cancer. In some embodiments, the sample comprises a cell from a tissue at high risk of developing cancer. In one embodiment, the sample comprises a cell from a tissue suspected to be cancerous.

Methods for detecting a reduced expression or activity of one or more poly-D/E protein comprise any method that interrogates a gene or its products at either the nucleic acid or protein level. Such methods are well known in the art and include, but are not limited to, nucleic acid hybridization techniques, nucleic acid reverse transcription methods, and nucleic acid amplification methods, western blots, northern blots, southern blots, ELISA, immunoprecipitation, immunofluorescence, flow cytometry, immunocytochemistry. In particular embodiments, disrupted gene transcription is detected on a protein level using, for example, antibodies that are directed against specific proteins. These antibodies can be used in various methods such as Western blot, ELISA, immunoprecipitation, flow cytometry, or immunocytochemistry techniques.

### Methods of Manufacturing Recombinant Protein

In certain embodiments, the present invention provides a method of using one or more poly-D/E protein in the production of a recombinant protein of interest. It is recognized in the art the recombinant proteins can spontaneously misfold and aggregate, thus reducing their functionality and utility. Thus, the one or more poly-D/E protein can be used to disaggregate protein aggregates of the recombinant protein of interest, thereby allowing for the production and collection of the functional recombinant protein of interest.

In certain embodiments, the present invention provides a method of increasing production of a recombinant protein of interest using one or more poly-D/E protein disclosed herein. It is recognized in the art that proteins, when overexpressed in cell-based expression systems, can misfold and aggregate at high concentrations resulting in premature cell death. Thus, proteins that can prevent or address protein misfolding, such as poly-D/E proteins of the present disclosure, can be used to prevent misfolding and cell death while allowing increased production of functional recombinant protein.

In certain embodiments, the method comprises administering to a cell one or more poly-D/E protein, a nucleic acid molecule encoding one or more poly-D/E protein, or a combination thereof. In certain embodiments, the cell is modified to express the recombinant protein of interest. The cell may be of any expression system, including, but not limited to a yeast expression system, bacterial expression system, insect expression system, or mammalian expression system.

### Methods of Cell Maintenance

In one embodiment, the present invention comprises a method of cell maintenance for use in cell therapy. It is recognized that cells for use in cell therapy, such as cells engineered to overexpress a therapeutic protein, may be subject to protein misfolding and aggregation leading to premature cell death. Thus, the poly-D/E proteins of the present disclosure can be used to prevent or address protein misfolding and aggregation to keep cells healthy and available for use. In one embodiment, the method comprises administering to said cell one or more poly-D/E protein, a nucleic acid molecule encoding one or more poly-D/E protein, or a combination thereof.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: DAXX Represents a New Type of Protein-Folding Enabler

DAXX, a polyD/E protein implicated in diverse cellular processes (Yang, X., et al., Cell 89, 1067-1076, 1997; Chang, H. Y., et al., Science 281, 1860-1863, 1998; Perlman, R., et al., Nat Cell Biol 3, 708-714, 2001; Zhao, L. Y., et al., J Biol Chem 279, 50566-50579, 2004; Tang, J., et al., Nat Cell Biol 8, 855-862, 2006; Lewis, P. W., et al., Proc Natl Acad Sci USA 107, 14075-14080, 2010; Song, M. S., et al., Nature 455, 813-817, 2008; Mahmud, I. & Liao, D., Nucleic Acids Res 47, 7734-7752, 2019), was initially identified as an adaptor protein associated with the intracellular death domain of the apoptosis receptor Fas (also known as CD95/Apo-1) (Yang, X., et al., Cell 89, 1067-1076, 1997; Chang, H. Y., et al., Science 281, 1860-1863, 1998). It was subsequently implicated in additional apoptotic scenarios and a wide range of other cellular processes (Perlman, R., et al., Nat Cell Biol 3, 708-714, 2001; Zhao, L. Y., et al., J Biol Chem 279, 50566-50579, 2004; Tang, J., et al., Nat Cell Biol 8, 855-862, 2006; Lewis, P. W., et al., Proc Natl Acad Sci USA 107, 14075-14080, 2010; Song, M. S., et al., Nature 455, 813-817, 2008; Mahmud, I. & Liao, D., Nucleic Acids Res 47, 7734-7752, 2019). Deficiency of Daxx results in embryonic lethality in mice (Michaelson, J. S., et al., Genes Dev 13, 1918-1923; 1999), whereas recurrent somatic mutations in DAXX are associated with human tumors (Jiao, Y., et al., Science 331, 1199-1203, 2011; Gopal, R. K., et al., Cancer Cell 34, 242-255, 2018). Although in most cases a specific mechanism has been proposed for its action, the association of DAXX with numerous cellular proteins raises an intriguing question as to whether DAXX possesses a biochemical activity that underlies, or contributes to, its remarkably diverse functions. It was therefore reasoned that if such a unifying activity exists for DAXX, it might be related to protein folding.

Herein, it is shown that DAXX possesses several protein-folding activities. DAXX prevents aggregation, solubilizes pre-existing aggregates, and unfolds misfolded species of model substrates and neurodegeneration-associated proteins. Notably, DAXX effectively prevents and reverses aggregation of its in vivo-validated client proteins, the tumor suppressor p53 and its principal antagonist MDM2. DAXX can also restore native conformation and function to tumour-associated, aggregation-prone p53 mutants, reducing their oncogenic properties. These DAXX activities are ATP-independent and instead rely on the polyD/E region. Other polyD/E proteins including ANP32A and SET can also function as stand-alone, ATP-independent molecular chaperones, disaggregases, and unfoldases. Thus, polyD/E proteins probably constitute a multifunctional protein quality control system that operates via a distinctive mechanism.

### Results

### DAXX is an Effective Molecular Chaperone

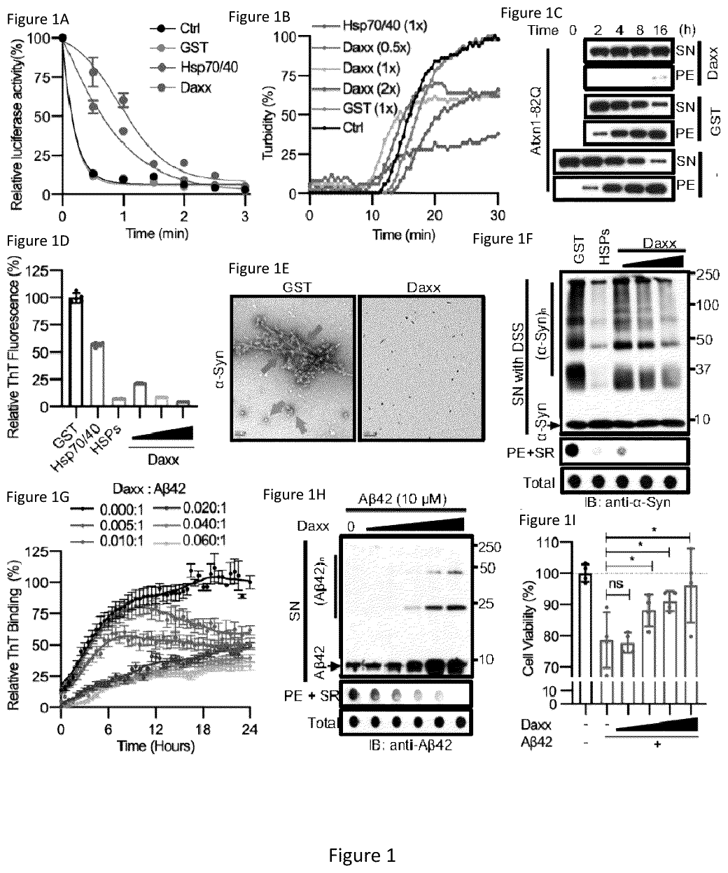

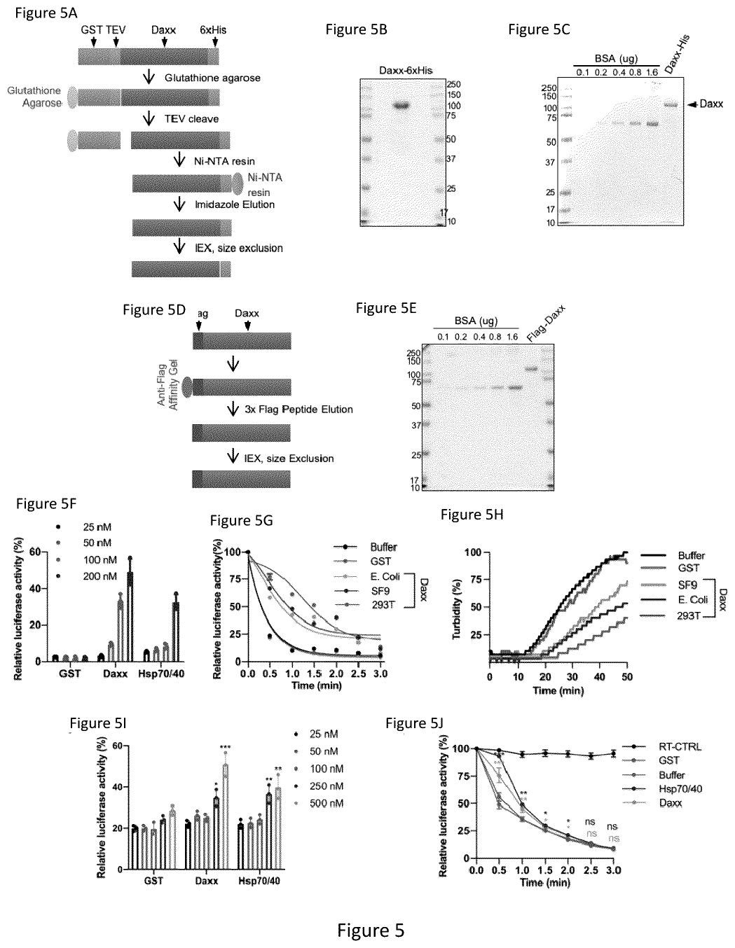

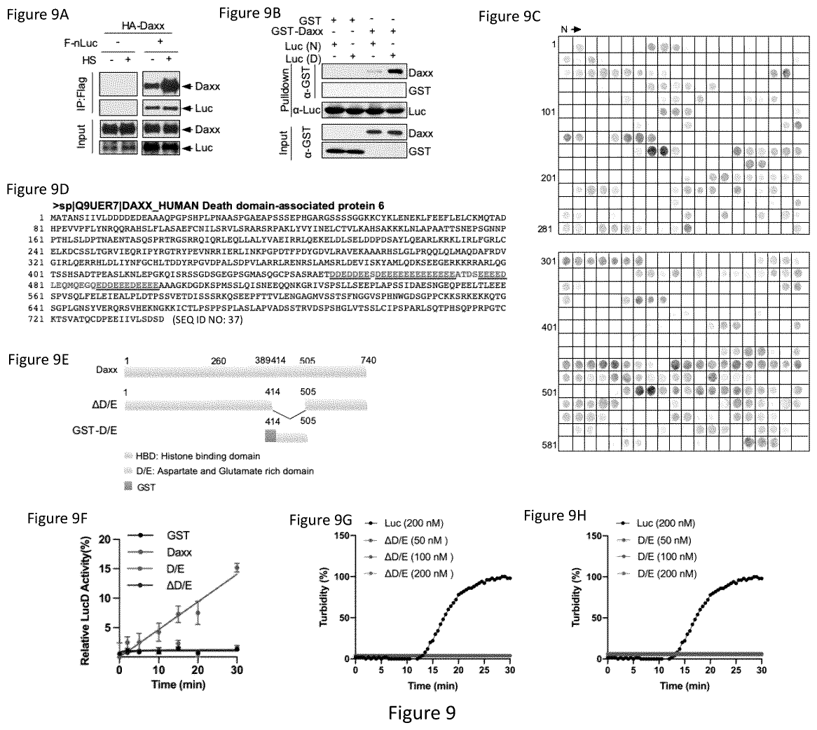

Molecular chaperones inhibit protein misfolding and aggregation (Balchin, D., et al., Science 353, aac4354, 2016). To investigate whether DAXX can act as a molecular chaperone, recombinant full-length DAXX protein was purified from bacterial, insect, and mammalian cells (FIGS. 5A-5E) and tested it on the model chaperone substrate luciferase and neurodegeneration-associated, misfolding-prone proteins. When luciferase was incubated at an elevated temperature, it lost enzymatic activity rapidly and coalesced into aggregates detectable by light scattering. DAXX purified from bacteria protected luciferase from heat-induced inactivation (FIGS. 1A and 9F) and aggregation (FIG. 1bB), akin to Hsp70 together with its co-chaperone Hsp40. DAXX proteins purified from insect Sf9 and human HEK293T cells also protected luciferase against thermal denaturation (FIGS. 5G-5J).

Neurodegenerative disease-associated proteins can spontaneously assemble into aggregated species including amyloid fibrils (Knowles, T. P., et al., Nat Rev Mol Cell Biol 15, 384-396, 2014). When incubated in vitro, ataxin-1 protein with an expanded polyglutamine tract (Atxn1 82Q), which is associated with spinocerebellar ataxia type 1 (SCA1), formed pelletable aggregates that were soluble with SDS (PE). DAXX strongly prevented the aggregation of Atxn1 82Q, keeping virtually all Atxn1 82Q molecules in the supernatant (SN) (FIG. 1C).

Highly-ordered amyloid fibrils, which consist of β-strands that are stacked perpendicularly to the fibril axis (cross-β structure), are a pathological hallmark of neurodegenerative diseases including Parkinson's disease (PD) and Alzheimer's disease (AD) (Knowles, T. P., et al., Nat Rev Mol Cell Biol 15, 384-396, 2014). DAXX inhibited fibrillization of PD-associated protein α-synuclein (a-Syn), as shown by thioflavin T (ThT)-binding assay (FIG. 1D), electron microscopy (EM) (FIG. 1E), and a dot blot assay that detected SDS-resistant (SR) as well as PE aggregates (FIG. 1F). A small amount of DAXX (0.1-0.4 μM) was sufficient to prevent aggregation of α-Syn monomers that were ˜175 to 700-fold in molar excess (70 μM). This activity of DAXX appeared to be stronger than that of Hsp70/Hsp40 and on a par with that of Hsp70/Hsp40 plus Hsp104^{A503S}, a potentiated version of the yeast disaggregase Hsp104 (Jackrel, M. E., et al., Cell 156, 170-182, 2014).

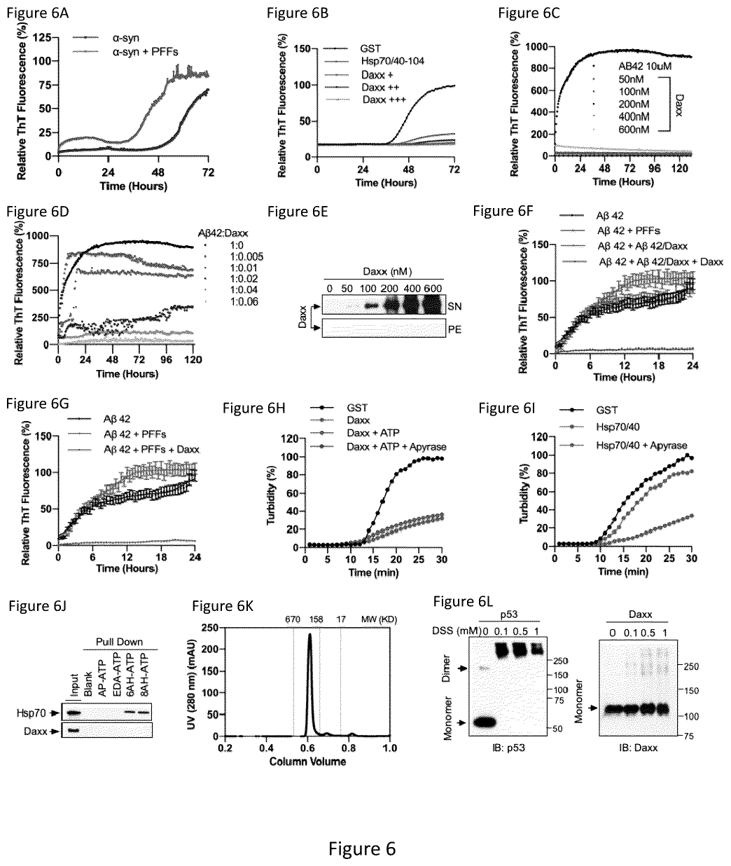

Amyloid fibrils can propagate in a prion-like, self-templating manner, a property that likely underlies the spread of fibrillar aggregates along interconnected neuronal regions in patients (Jucker, M. & Walker, L. C., Nature 501, 45-51, 2013). Aggregation of soluble α-Syn monomers was accelerated by preformed fibrils (PFFs) of α-Syn (FIG. 6A). DAXX suppressed this seeded fibrillization at sub-stoichiometric molar ratios to α-Syn monomers in a dose-dependent manner and near-completely blocked it at a relatively high dose (FIG. 6B).

To further assess the effect of DAXX on protein fibrillization, a substrate with a stronger propensity to aggregate, the AD-associated amyloid-beta peptide Aβ42, was used. DAXX inhibited fibrillization of Aβ42 at low molar ratios (1:200 to 1:17), maintaining it in a soluble state even after a prolonged incubation (FIGS. 1G, 1H, and 6C-6E). Consequently, in the presence of DAXX, Aβ42 monomers could not form PFFs that accelerated aggregation of fresh Aβ42 monomers (FIG. 6F). Moreover, DAXX nearly-completely abolished Aβ42 PFFs-induced aggregation of fresh Aβ42 monomers (FIG. 6G). Therefore, DAXX suppresses both spontaneous and seeded aggregation of disease-associated proteins.

Preceding fibrillization, α-Syn and Aβ42 monomers form soluble oligomers that are neurotoxic (Kayed, R., et al., Science 300, 486-489, 2003). DAXX blocked the formation of α-Syn oligomers of various sizes, akin to Hsp70/Hsp40-Hsp104^{A503S} (FIGS. 1E and 1F). Moreover, while Aβ42 peptides pre-incubated alone were toxic to human neuroblastoma SH-SY5Y cells, Aβ42 peptides pre-incubated with DAXX displayed minimal toxicity (FIG. 1I). Therefore, DAXX prevents the formation of toxic prefibrillar oligomers.

Unlike canonical chaperones, the activity of DAXX did not require the addition of ATP (FIGS. 1A-1H, 5F-5J, and 6B-6H); nor was it affected by the treatment of the ATP-diphosphohydrolase apyrase (FIGS. 6H and 6I). DAXX was unable to bind to ATP (FIG. 6J). Canonical molecular chaperones often assemble into a dimer or a large oligomeric complex (Balchin, D., et al., Science 353, aac4354, 2016). In contrast, size exclusion chromatography and chemical crosslinking studies suggested that DAXX exists predominantly as a monomer (FIGS. 6K and 6L).

### DAXX is a Protein Disaggregase

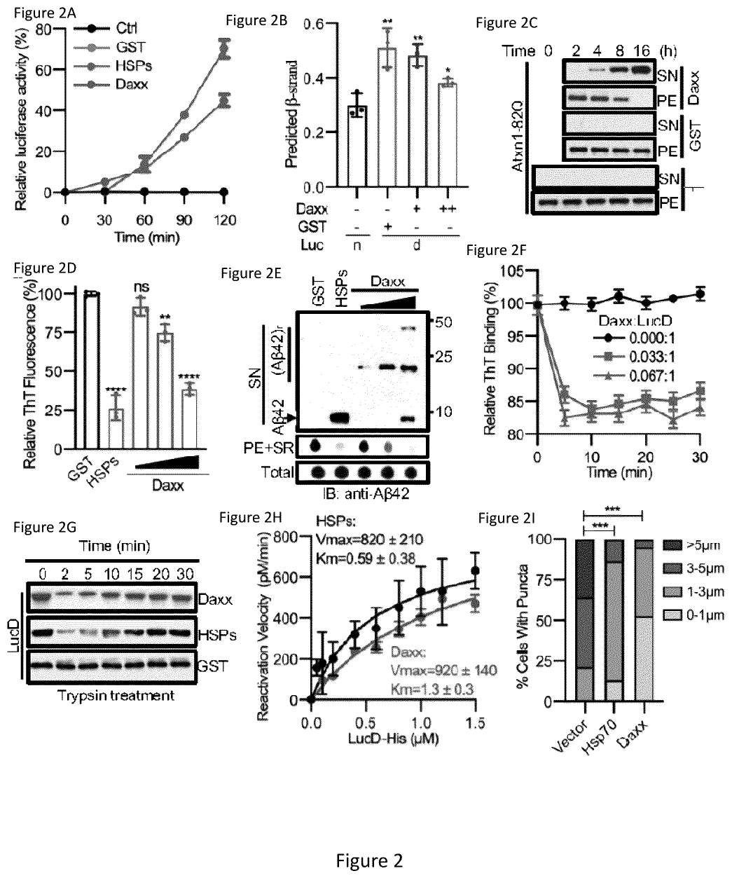

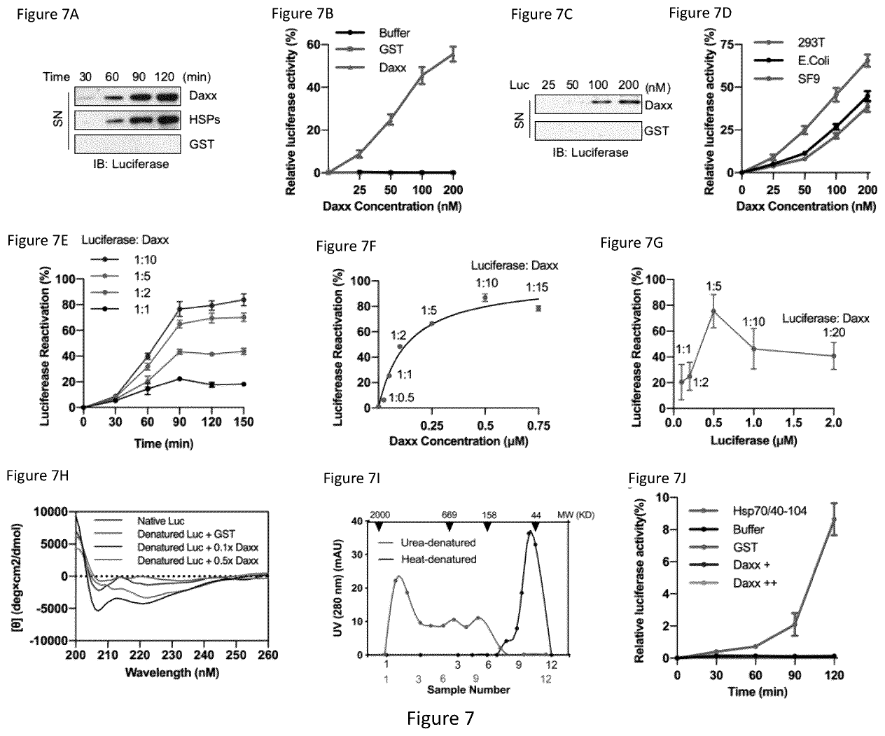

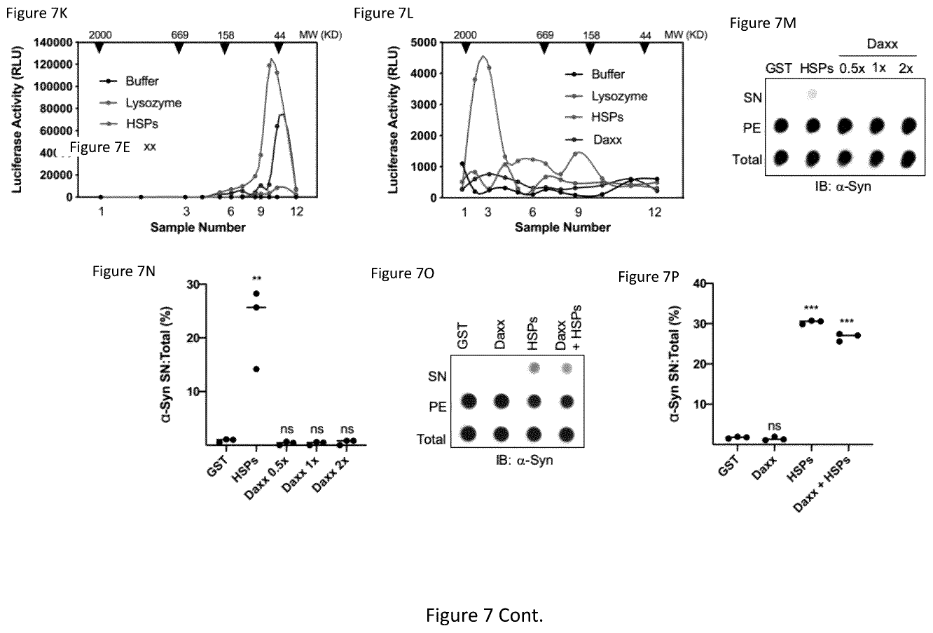

Disaggregases dissolve pre-existing protein aggregates, permitting refolding of misfolded proteins and hence avoiding the energetically costly process of protein degradation and re-synthesis (Saibil, H., Nat Rev Mol Cell Biol 14, 630-642, 2013). DAXX protein purified from bacteria was able to dissolve luciferase aggregates generated by thermal denaturation and reactivate them in a time- and dose-dependent manner (FIGS. 2A and 7A-C). DAXX proteins purified from Sf9 and HEK293T cells exhibited a similar ability (FIGS. 7D-7F). DAXX achieved a maximal recovery of luciferase activity at five-fold excess (FIG. 7G). A circular dichroism (CD) spectroscopic analysis showed DAXX reduced the β-strand content of heat-treated luciferase to a level close to that of unheated luciferase (FIGS. 2B and 7H), indicating that DAXX returns the core structure of denatured luciferase to a nearly native state.

While luciferase formed aggregates of relatively small sizes upon heat treatment, it generated aggregates of large sizes upon urea treatment (FIG. 7I) (Glover, J. R. & Lindquist, S., Cell 94, 73-82, 1998). DAXX exhibited little activity towards urea-produced luciferase aggregates, whereas Hsp70/Hsp40-Hsp104^{A503S} showed a modest activity (FIGS. 7J-7L). When tested on disease-associated proteins, DAXX exhibited potent activity towards some, but not other, aggregates. DAXX readily disassembled the amorphous Atxn1 82Q aggregates (FIG. 2C) and could also convert virtually all Aβ42 fibrils into a soluble state (FIGS. 2D and 2E). However, DAXX was unable to dissolve a-Syn fibrils by itself; nor did it synergize with Hsp70/Hsp40-Hsp104^{A503S} for disaggregation (FIGS. 7M-7P). As for its chaperone activity, the disaggregase activity of DAXX was independent of ATP (FIGS. 2A-2E and 7A-7P).

### DAXX is a Protein Unfoldase

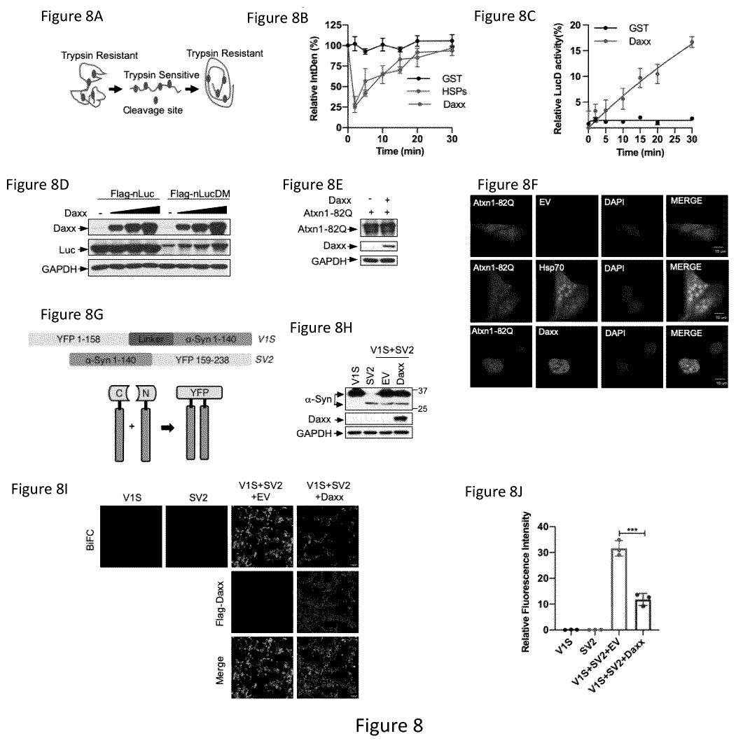

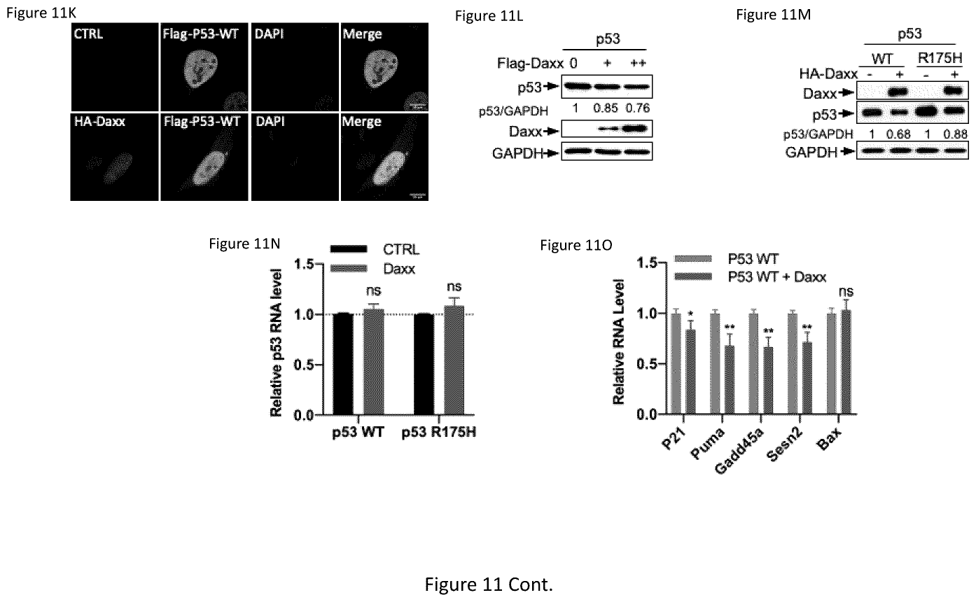

Unfoldases can release stable misfolded monomers from kinetically-trapped states, a property previously shown for the Hsp70 chaperone system (Jackrel, M. E., et al., Cell 156, 170-182, 2014; Sharma, S. K., et al., Nat Chem Biol 6, 914-920, 2010). To test whether DAXX possesses unfoldase activity, we used the luciferase mutant LucD, which adapts a compact, monomeric misfolded state upon repeated freeze-thaw cycles[[²⁰]]. Misfolded LucD monomers binds more ThT than native LucD, reflecting a high β-sheet content (Sharma, S. K., et al., Nat Chem Biol 6, 914-920, 2010). DAXX decreased the binding of misfolded LucD monomers to ThT, consistent with unfoldase activity (FIG. 2F). Sensitivity to brief trypsin digestion is another indicator for the unfolded state (FIG. 11A)²⁰. Incubation with DAXX for a short period of time (2 min) enhanced the sensitivity of LucD to trypsin, suggesting rapid unfolding of the compact LucD monomers, whereas a longer incubation with DAXX (5-30 min) progressively reduced LucD sensitivity to trypsin while increasing its enzymatic activity (FIGS. 2G, 8B, and 8C), indicating the refolding of LucD to the native state. These effects of DAXX were similar to those of Hsp70/Hsp40-Hsp104^{A503S}. Moreover, reactivation of LucD in the presence of DAXX followed saturation kinetics with apparent Vₘₐₓ′ and Kₘ′ comparable to those in the presence of Hsp70/Hsp40-Hsp104^{A503S} (FIG. 2H). Collectively, these results suggest that DAXX serves as a catalyst to refold misfolded monomers.

### Effect of DAXX in Cells

To evaluate the effect of DAXX on protein aggregation in cells, it was co-expressed with a nucleus-localized luciferase (nLuc) or its structurally destabilized derivative (nLucDM) (Gupta, R., et al., Nat Methods 8, 879-884, 2011) in HEK293T cells. DAXX elevated the levels of nLucDM, but not nLuc, in a dose-dependent manner (FIG. 8D). Moreover, in U2OS cells, DAXX reduced the size and number of Atxn1 82Q inclusions, with an effect stronger than that of HSP70 (FIGS. 2I, 8E, and 8F).

To further assess the effect of DAXX on oligomeric intermediates, a bimolecular fluorescence complementation (BiFC) system was used in which α-Syn was fused to the N-terminal (V1) or the C-terminal (V2) fragment of the Venus protein (FIG. 8G) (Outeiro, T. F., et al., PLOS One 3, e1867, 2008). When V1-a-Syn (V1S) and α-Syn-V2 (SV2) were expressed together, but not individually, reconstitution of the Venus fluorescence occurred (FIGS. 8H-8J), reflecting α-Syn oligomerization that brought the split Venus moieties into proximity. DAXX markedly reduced the BiFC signal, but not VIS and SV2 protein levels (FIGS. 8H-8J). Together, these results indicate that DAXX suppresses the formation of aggregates and prefibrillar oligomers in cells.

Role of the polyD/E Domain

The various activities of DAXX in assisting protein folding suggested an intrinsic ability to recognize misfolded conformations. Consistently, when DAXX and nLuc were co-expressed in HEK293T cells, their interaction was increased upon heat shock (FIG. 9A). In vitro, DAXX preferentially bound to heat-denatured over native luciferase (FIG. 9B), indicating that DAXX can distinguish misfolded and native conformers of the same polypeptide.

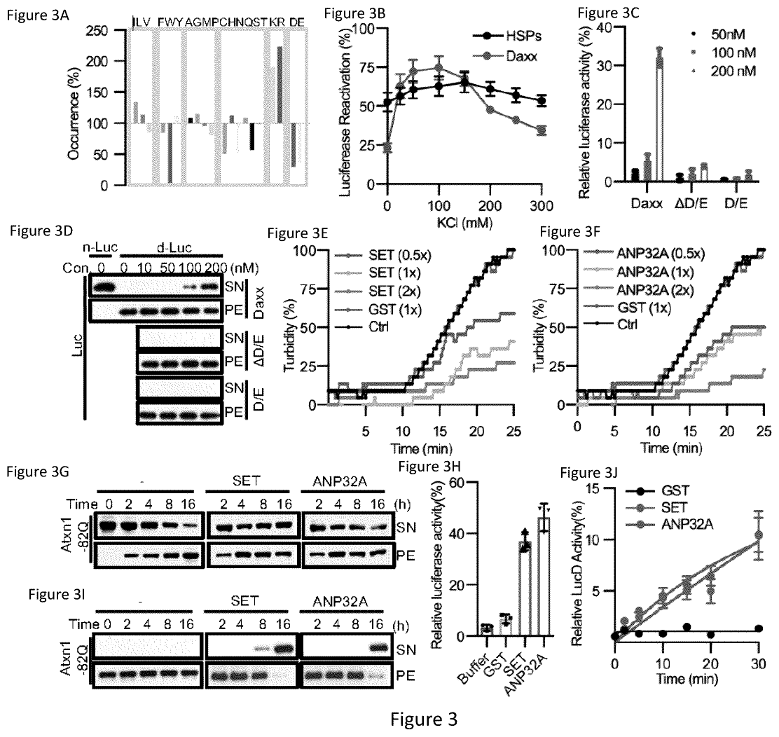

Canonical molecular chaperones and disaggregases can recognize linear peptide segments of unfolded proteins that are enriched in hydrophobic amino acids (Balchin, D., et al., Science 353, aac4354, 2016). To define the molecular basis by which DAXX recognizes misfolded proteins, a cellulose-bound peptide library was generated consisting of peptides derived from luciferase, four physiologically-relevant client proteins (p53, MDM2, H3.3, and H4) (Zhao, L. Y., et al., J Biol Chem 279, 50566-50579, 2004; Tang, J., et al., Nat Cell Biol 8, 855-862, 2006; Lewis, P. W., et al., Proc Natl Acad Sci USA 107, 14075-14080, 2010), and DAXX itself. DAXX bound to a small subset of this library (FIG. 9c), indicating its ability to discern peptides with different amino acid compositions. An analysis of the relative occurrence of each amino acid residue in DAXX-interacting peptides versus that in all peptides of the library revealed that DAXX strongly favored basic residues Arg and Lys and, to a lesser extent, hydrophobic residues Ile and Leu, while disfavoring acidic residues Asp and Glu; polar residues Cys, Asn, and Ser; and the aromatic residue Trp (FIG. 3A). Therefore, DAXX likely recognizes misfolded proteins in part through electrostatic interactions.

To test this notion, the activity of DAXX to recover denatured luciferase was examined in the presence of increasingly higher salt concentrations (0-300 mM KCl). The activity of DAXX initially strengthened (0-25 mM), reached a maximum (25-150 mM), and then progressively declined (150-300 mM) (FIG. 3B). In contrast, the activity of Hsp70/Hsp40-Hsp104^{A503S} remained largely unchanged (FIG. 3B). The decrease in DAXX activity at high ionic strength is consistent with the involvement of electrostatic interactions. But the initial increase in, and the subsequent maintenance of, its activity distinguish DAXX from polyanions such as nucleic acids, which show a monotonical decrease in activity with increasing salt (Rentzeperis, D., Jonsson, T. & Sauer, R. T., Nat Struct Biol 6, 569-573, 1999). Thus, DAXX might utilize electrostatic interactions in a regulated manner.

Of note, DAXX contains a region of mainly Asp and Glu (FIG. 9D) (Yang, X., et al., Cell 89, 1067-1076, 1997). Mutants lacking this polyD/E region (ΔD/E) or consisting mostly of it (D/E) were generated (FIG. 9E). DAXX ΔD/E did not protect luciferase from heat inactivation (FIG. 3C), solubilize luciferase aggregates (FIG. 3D), or unfold LucD monomers (FIGS. 9F-9H); nor did DAXX D/E. Thus, the polyD/E region of DAXX is necessary, albeit insufficient, for various protein-folding activities.

Activity of Other polyD/E Proteins

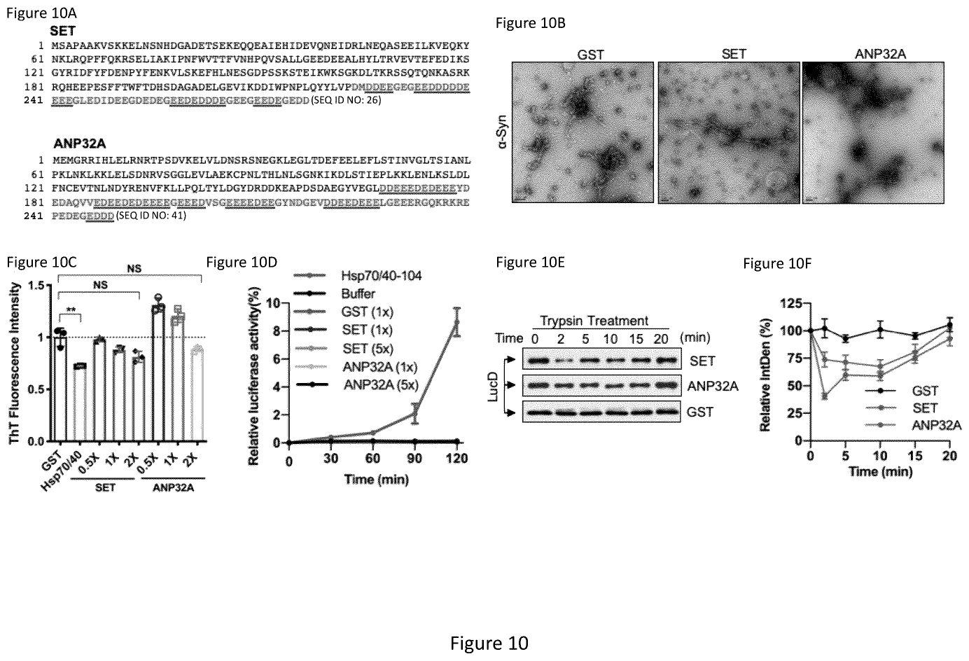

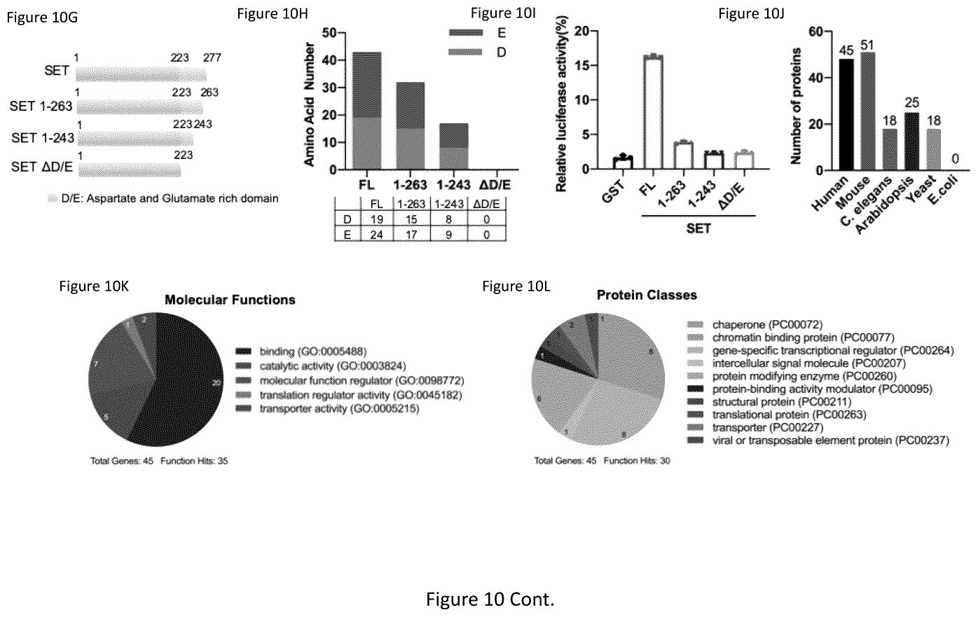

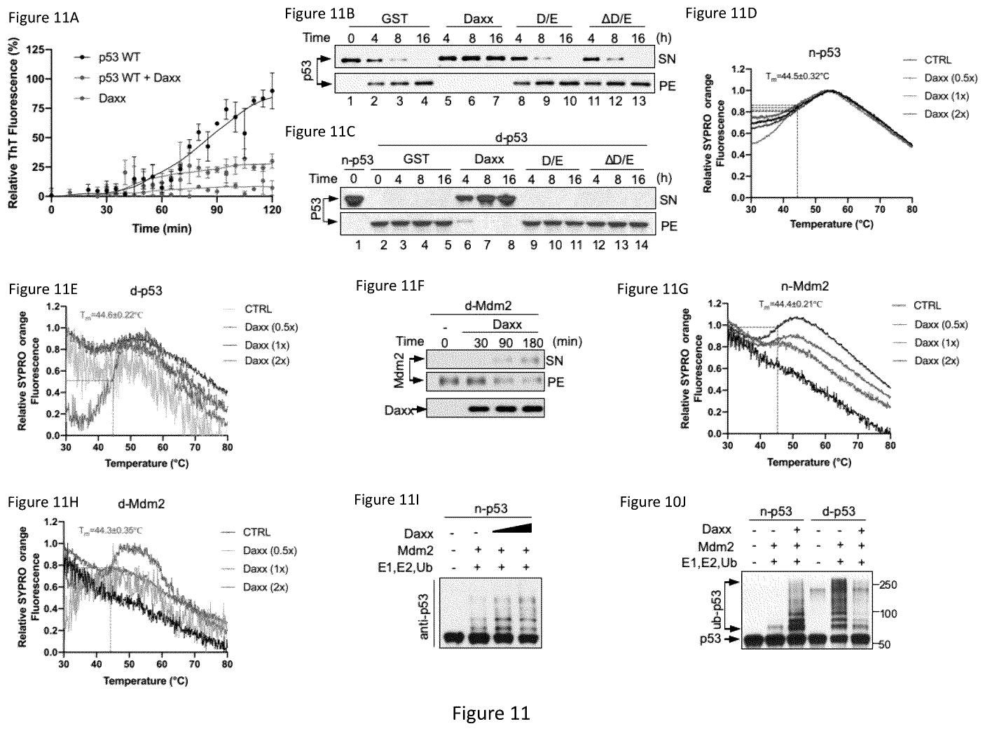

Proteins containing an extended polyD/E region with one or more continuous sequences of Asp and Glu (acidic runs) were first reported in the 1970s (Walker, J. M., et al., Nature 271, 281-282, 1978), and were subsequently found in various eukaryotes (Karlin, S., et al., Proc Natl Acad Sci USA 99, 333-338, 2002). To investigate whether other polyD/E proteins can facilitate protein folding, we analyzed ANP32A and SET, both of which contain a polyD/E region at their C-terminal regions (FIG. 10A) (Adachi, Y., et al., J Biol Chem 269, 2258-2262, 1994; Vaesen, M., et al., Biol Chem Hoppe Seyler 375, 113-126, 1994). Recombinant ANP32A and SET proteins protected luciferase against heat-induced aggregation (FIGS. 3E and 3F) and prevented Atxn1 82Q from spontaneous aggregation (FIG. 3G). Unlike DAXX, however, ANP32A and SET did not block α-Syn fibrillization (FIGS. 10B and 10C).

ANP32A and SET were also capable of reactivating heat-denatured luciferase (FIG. 3H) and dissolving Atxn1 82Q aggregates (FIG. 3I). Similar to DAXX, they were unable to reactivate urea-denatured luciferase (FIG. 10d) or α-Syn fibrils (data not shown). ANP32A and SET could release misfolded LucD monomers from the energetically-trapped state and facilitate their re-folding (FIGS. 3I, 10E, and 10F). Removing 14 amino acids or more from the SET polyD/E region dramatically reduced its ability to reactivate luciferase (FIGS. 10G-101), suggesting that the majority of polyD/E region is required for optimal activity.

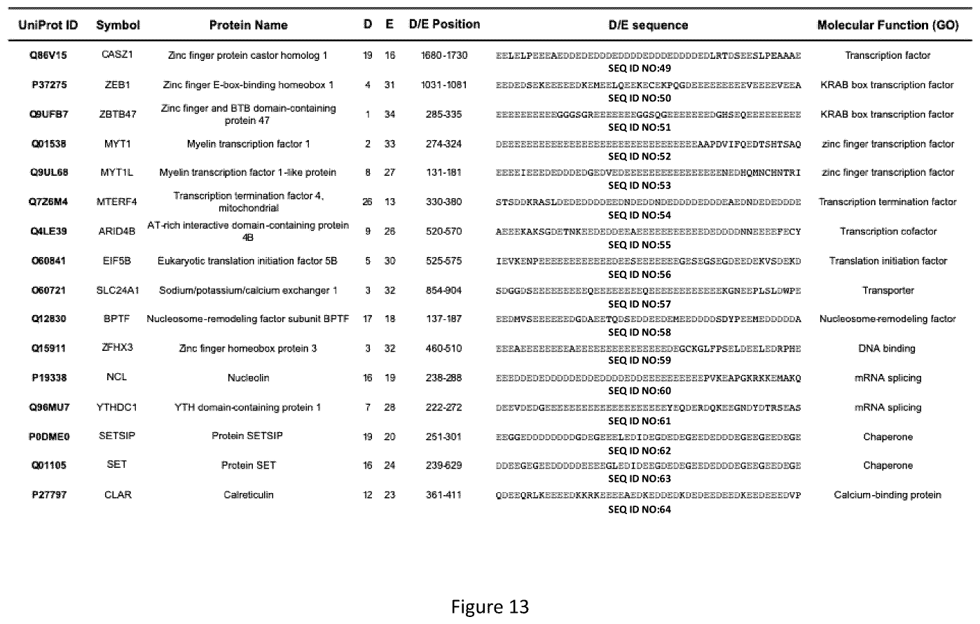

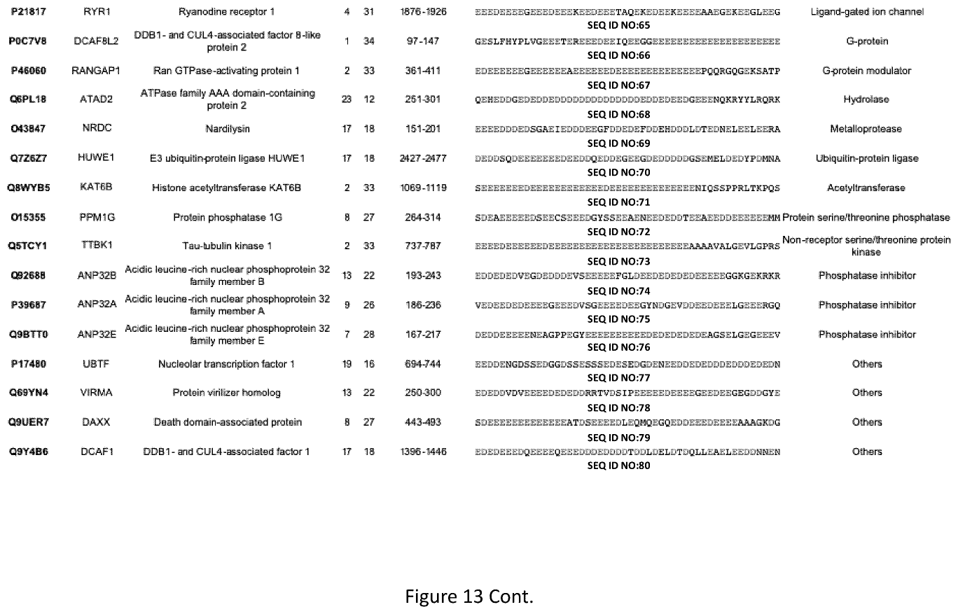

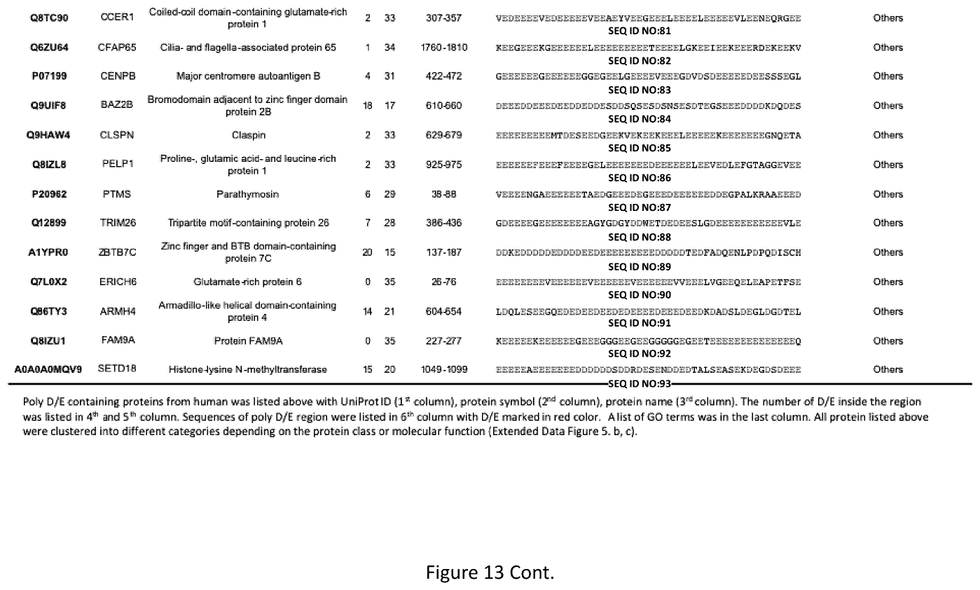

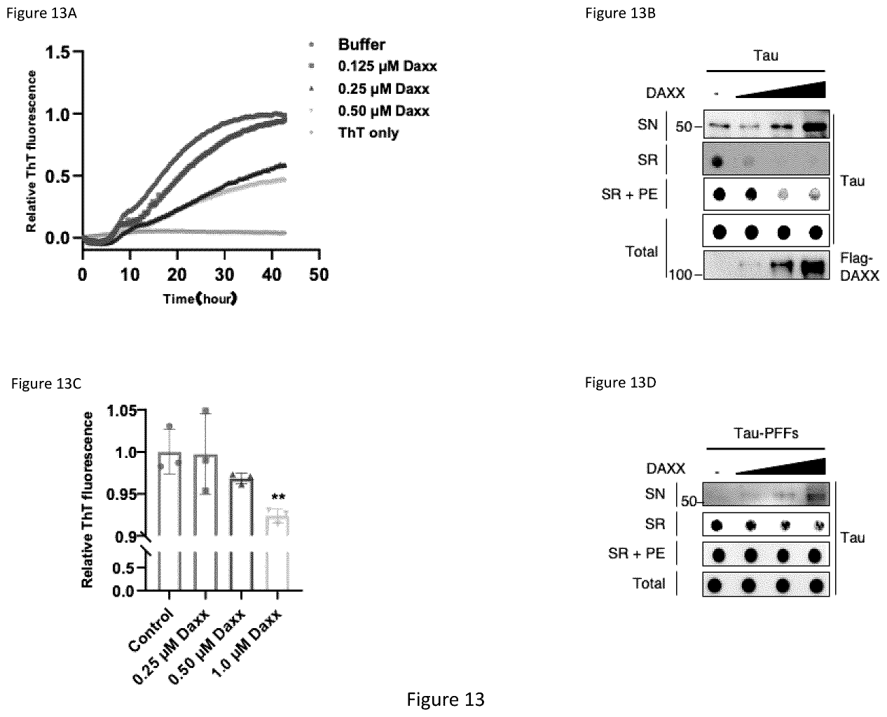

PolyD/E proteins were previously surveyed based on relatively long acidic runs (Karlin, S., et al., Proc Natl Acad Sci USA 99, 333-338, 2002; Wang, D. et al., Nature 538, 118-122, 2016). An analysis of polyD/E domains in DAXX, ANP32A, and SET showed that the occurrence of Asp and Glu residues is equal to, or greater than, 35 in a 50-amino acid window. Using this criteria, various proteomes were searched and a sizable number of polyD/E domain proteins in metazoans were identified including 45 in humans and 51 in mice. These proteins also exist in Arabidopsis (25) and S. cerevisiae (18), but not in E. coli. (FIGS. 10J and 13). Gene ontology analysis showed that human polyD/E proteins are involved in various cellular processes (FIGS. 10K and 10L). The precise number of these proteins requires additional analysis of the composition of the polyD/E domain that contributes to its activity. Nevertheless, polyD/E proteins appear to be prevalent in eukaryotic genomes, and their number has expanded significantly during evolution.

### DAXX Chaperones Folding of p53 and MDM2

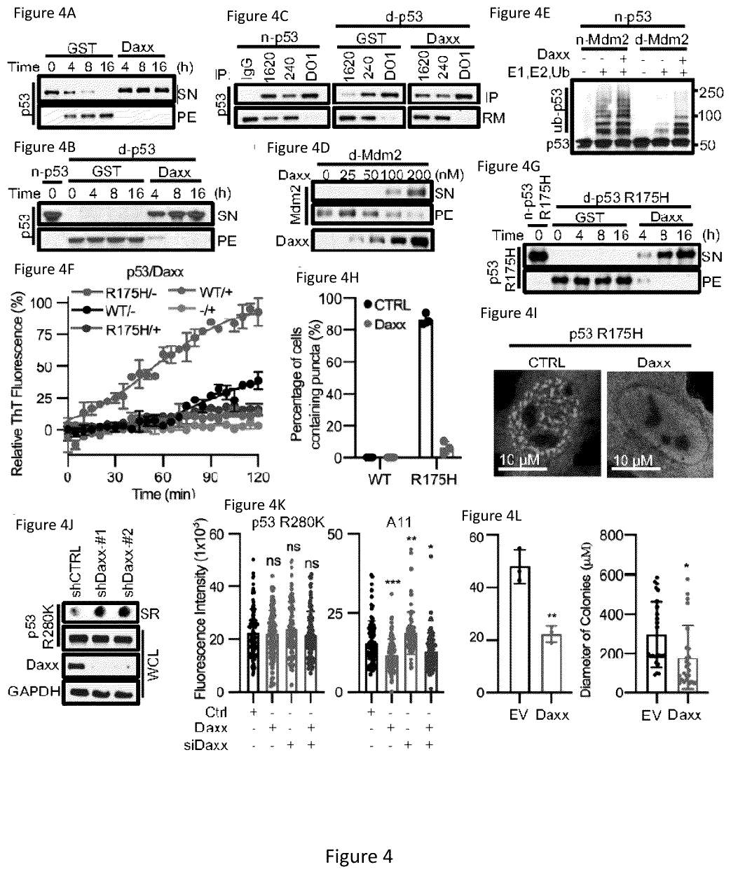

To evaluate whether polyD/E proteins promote the folding of their in vivo-validated client proteins, the effect of DAXX on p53 and its ubiquitin ligase MDM2 were examined (Zhao, L. Y., et al., J Biol Chem 279, 50566-50579, 2004; Tang, J., et al., Nat Cell Biol 8, 855-862, 2006). p53 is a highly labile protein, and purified recombinant p53 readily misfolds and aggregates (Butler, J. S. & Loh, S. N., Protein Sci 15, 2457-2465, 2006). DAXX blocked p53 aggregation, keeping virtually all p53 molecules in the soluble form (FIG. 4A). p53 can also form amyloid fibrils (Ishimaru, D. et al., Biochemistry 42, 9022-9027, 2003; Ano Bom, A. P. et al., J Biol Chem 287, 28152-28162, 2012), which was abrogated by DAXX as well (FIG. 11A). Moreover, DAXX displayed potent disaggregase activity towards pre-existing p53 aggregates, converting virtually all of them back to the soluble state (FIG. 4B). In contrast, neither DAXX ΔD/E nor DAXX D/E exhibited chaperone and disaggregase activity towards p53 (FIGS. 11B and 11C).

Using antibodies specific to the wild-type (PAb1620) or mutant (PAb240) conformation of p53, we observed that DAXX restored misfolded p53 to its native conformation (FIG. 4C). Moreover, a thermal denaturation shift assay (Zhang, R. & Monsma, F., Curr Opin Drug Discov Devel 13, 389-402, 2010) showed that while DAXX did not significantly affect the transition temperature (Tₘ) of native p53, it elevated the Tₘ of denatured p53 to that of native p53 (FIGS. 11D and 11E).

As for p53, DAXX was able to solubilize MDM2 molecules from aggregates and restored their native conformation (FIGS. 4D, 11G, and 11H). DAXX enhanced native MDM2 (n-MDM2)-mediated ubiquitination of p53, and partially restored ligase activity to heat-treated MDM2 (d-MDM2) (FIGS. 4E and 11I). MDM2 ubiquitinated denatured p53 more readily than native p53. Pre-incubation with DAXX reduced ubiquitination of denatured p53 (FIG. 11J) again indicating that DAXX restores the native conformation of p53.

Consistent with its ability to promote MDM2-mediated p53 ubiquitination, DAXX reduced p53 protein levels in U2OS cells (FIGS. 11K and 11L). DAXX also decreased p53 protein levels in H1299 cells where p53 was inducibly expressed, and lowered expression of p53 target genes (FIGS. 11M-11O). Collectively, these results indicate that DAXX maintains the native conformation of both p53 and MDM2, enhancing the robustness of the p53-MDM2 regulatory network.

### Effect of DAXX on Mutant p53

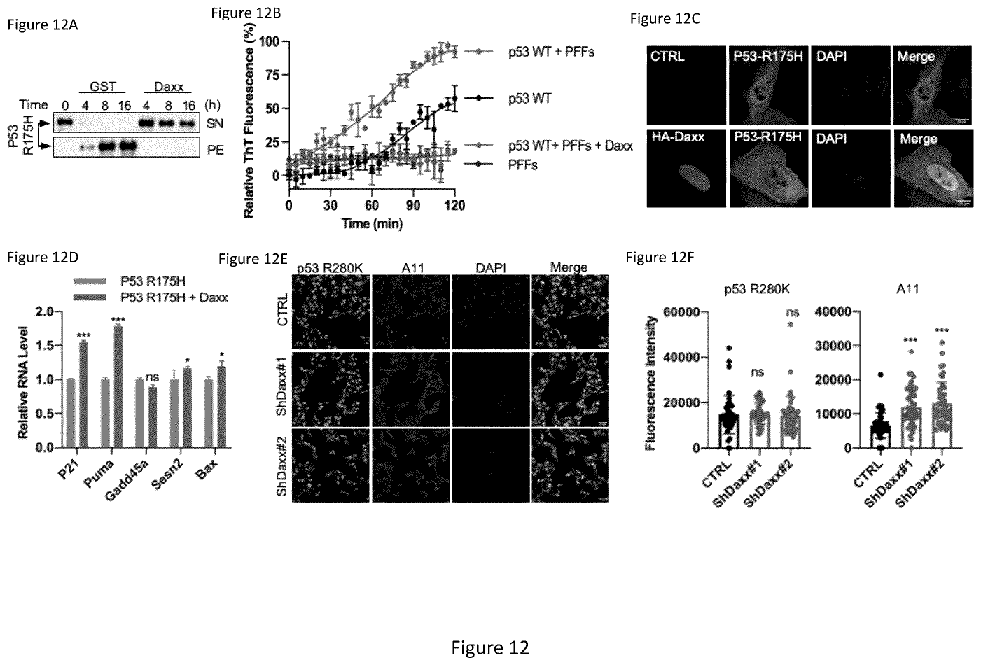

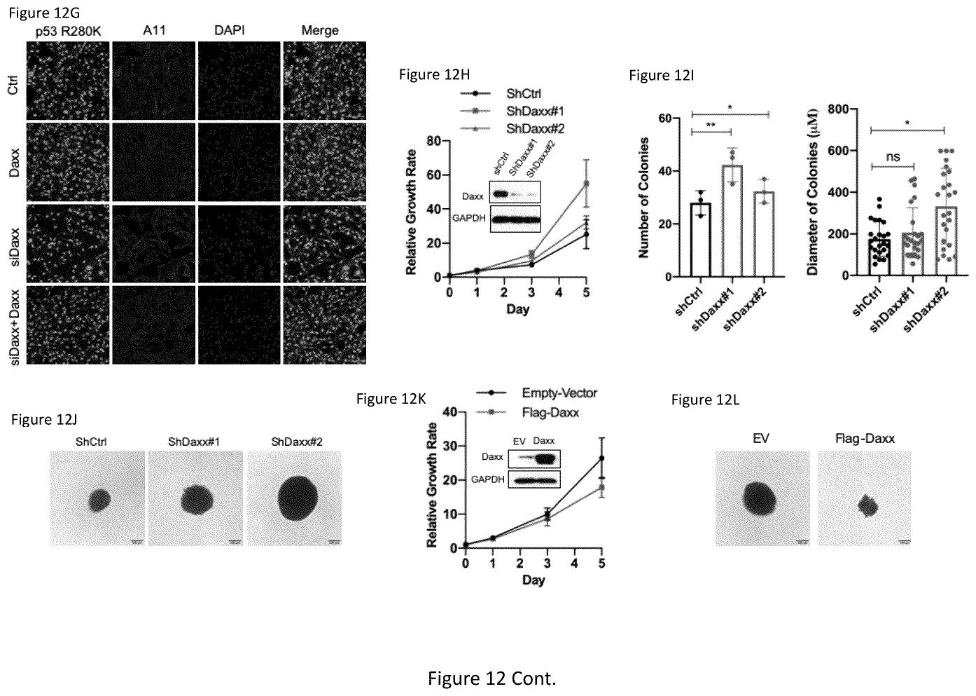

p53 is the most frequently mutated gene in human tumors (Muller, P. A. & Vousden, K. H., Cancer Cell 25, 304-317, 2014). A substantial fraction of tumor-associated mutations destabilize conformation of p53 protein and accelerate its aggregation, contributing to more aggressive tumor phenotypes. To investigate whether DAXX can rescue the function of mutant p53, a “hotspot” conformational mutation, R175H, was used. Compared to wild-type p53, p53^{R175H} aggregated at a faster pace. Still, DAXX nearly-completely prevented p53^{R175H} aggregation (FIG. 12A). p53^{R175H} more readily generated amyloid fibrils, which was again effectively blocked by DAXX (FIG. 4F). DAXX also rendered preformed p53^{R175H} PFFs incapable of inducing fibrillization of wild-type p53 (FIG. 12B). Furthermore, DAXX was able to transition nearly all pre-existing p53^{R175H} insoluble aggregates back into solution (FIG. 4G). In U2OS cells, DAXX strongly reduced p53^{R175H} aggregates that appeared as puncta (FIGS. 4H, 4I, and 12C). DAXX also reduced the protein, but not mRNA, levels of p53^{R175H} in H1299 cells that inducibly express this mutant and increased the expression of p53 target genes including p21 and Puma (FIGS. 11M, 11N, and 12D). These results suggest that DAXX converts p53^{R175H} to the native state, rendering it responsive to MDM2-mediated degradation and restoring its normal function.

To further examine the influence of DAXX on mutant p53 and the associated oncogenic phenotypes, breast cancer MDA-MB-231 cells, which harbored the conformational mutant p53^{R280K} that aggregates into amyloid fibrils, were chosen (Ano Bom, A. P. et al., J Biol Chem 287, 28152-28162, 2012). Knocking down DAXX by independent small hairpin RNAs (shRNAs) increased intracellular p53^{R280K} fibrillar aggregates (FIGS. 4J, 12E, and 12F). Knocking down DAXX by a small interfering RNA (siRNA) yielded a similar result (FIGS. 4K and 12G). In contrast, forced expression of siRNA-resistant DAXX not only reversed the effect of the DAXX siRNA but also further decreased p53^{R280K} aggregates (FIGS. 4K and 12G).

Knocking down DAXX increased proliferation of MDA-MB-231 cells on adherent plates (FIG. 12H), and enhanced their ability to grow in soft-agar medium (FIGS. 12I and 12J), an in vitro measure of tumorigenicity. Conversely, forced expression of DAXX impeded adherent proliferation of MDA-MB-231 cells (FIG. 12K) and reduced the number and size of soft-agar colonies formed by these cells by ˜50% and ˜40%, respectively (FIGS. 4L and 12L). Collectively, these data suggest that DAXX may restore the native conformation and function to aggregation-prone p53 mutants, reducing their oncogenic properties.

### DAXX Prevents Tau Aggregation

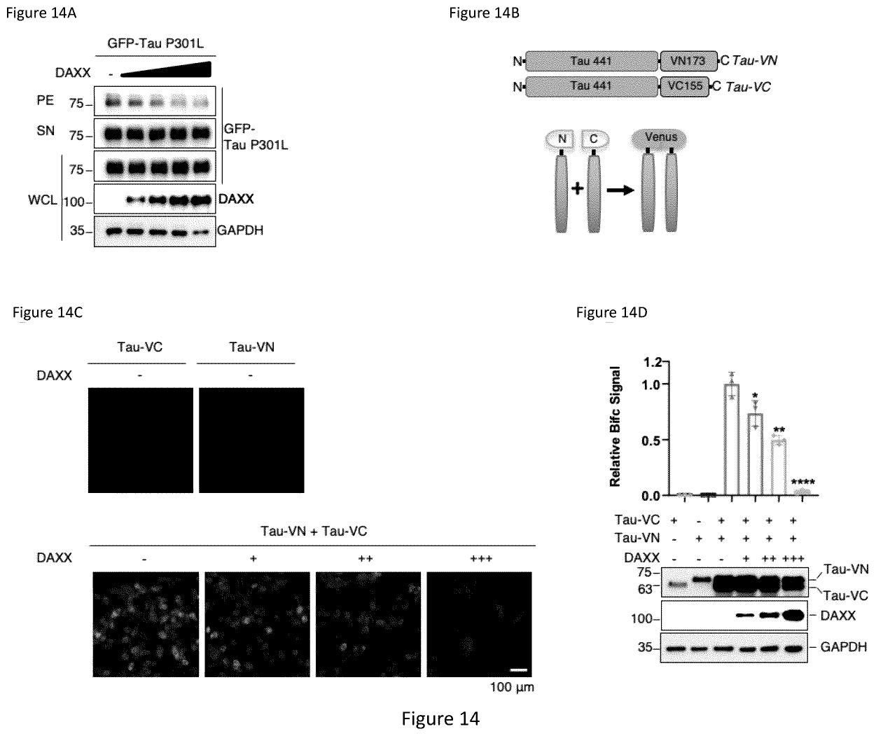

To determine applications in neurodegenerative diseases, the ability of DAXX to function as a molecular chaperone for Tau was investigated. When incubated in the presence of heparin, recombinant Tau protein spontaneously produced amyloid fibrils, as expected (Goedert, M. et al., Nature, 383:550-553, 1996). This was indicated by the increased binding to thioflavin T (ThT), an amyloid-specific dye (FIG. 14A), as well as by Western and dot blot assays that detected soluble Tau in the supernatant (SN) and SDS-soluble (PE) and SDS-resistant (SR) aggregates in the pellet (FIG. 14B). Flag-DAXX purified from HEK293T cells effectively prevented Tau fibrillization at low substoichiometric ratios, reducing it by ˜50% at a 1:20 or 1:10 molar ratio to Tau (FIG. 14A), keeping the majority of Tau molecules in a soluble state (FIG. 14B). This observation indicates that DAXX is a molecular chaperone for Tau, preventing its misfolding and aggregation. Unlike canonical chaperones such as the HSP70 and HSP90 systems, which are multicomponent machineries that are driven by energy derived from ATP hydrolysis (Balchin, D., et al., Science, 353: aac4354, 2016), DAXX prevents Tau aggregation alone and in the absence of ATP (FIGS. 14A and 14B).

### DAXX Dissolves Pre-Existing Tau Fibrils

When incubated with pre-existing Tau fibrils, Flag-DAXX was able to partially dissolve these aggregates, reducing their bindings to ThT (FIG. 14C) and converting a substantial portion of these aggregates to a soluble state (FIG. 14D). Thus, DAXX is also a disaggregase for Tau. As for its molecular chaperone activity, DAXX dissolves Tau aggregates without auxiliary factors or ATP.

DAXX Prevents Tau from Aggregating into Insoluble Fibrils and Soluble Oligomers in Cells

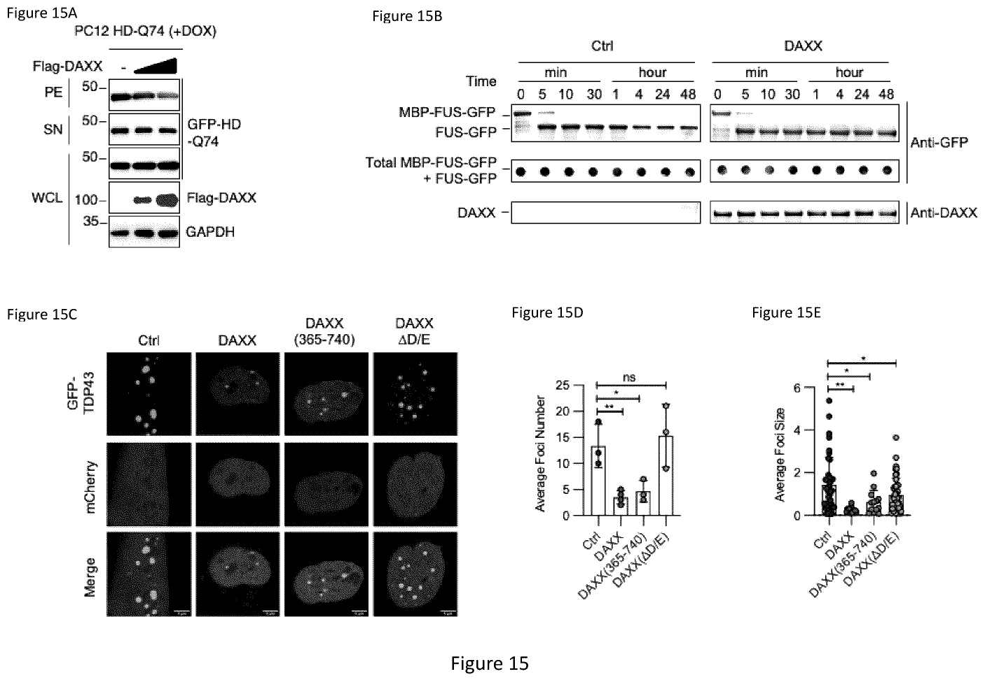

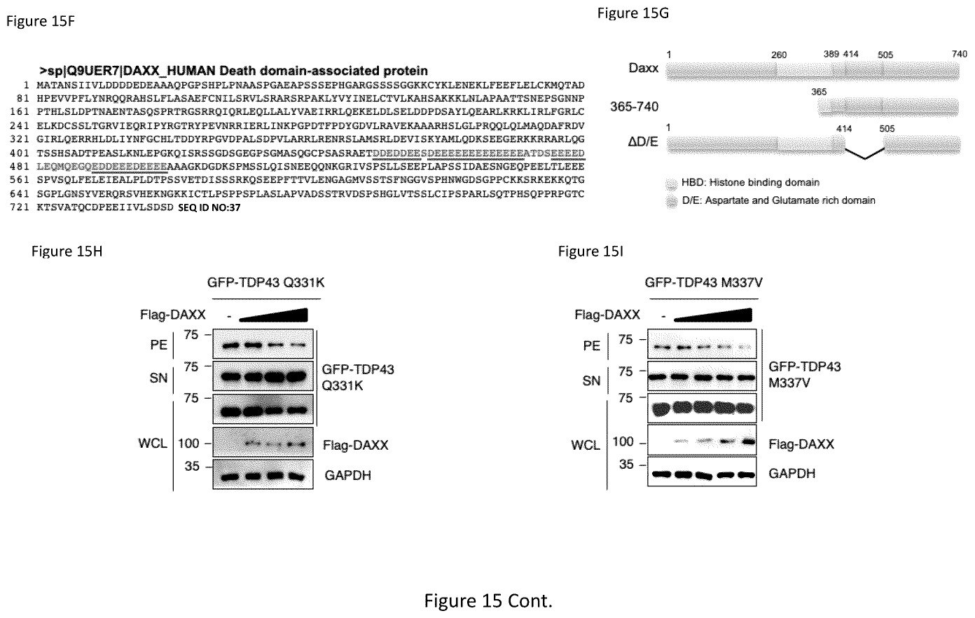

To examine the effect of DAXX on Tau aggregation in cells, an enhanced green fluorescence protein (GFP) fusion of the longest isoform of human Tau (2N4R) carrying the P301L mutation, which is associated with familial FTLD, was used (Dumanchin, C., et al., Human Molecular Genetics, 7:1825-1829, 1998; Hutton, M., et al., Nature, 393:702-705, 1998). When expressed alone HEK293T cells, GFP-Tau P301L alone formed aggregated species in cell lysates, which were detected in the insoluble pellet (PE) fraction in a sedimentation assay (FIG. 15A). DAXX reduced GFP-Tau P301L aggregates in a dose-dependent manner and was able to block the majority of the aggregates at a high dose. In contrast, DAXX did not affect the overall levels of GFP-Tau P301L (FIG. 15A), indicating that DAXX enhances the solubility of GFP-Tau P301L rather than promoting its degradation.

Preceding the formation of insoluble fibrillar aggregates, Tau, similar to other misfolding-prone proteins linked to neurodegeneration (Kayed, R., et al., Science, 300:486-489, 2003), assembles into soluble oligomeric species, which could be neurotoxic (Lasagna-Reeves, C. A., et al., Molecular Neurodegeneration, 6:39, 2011). To assess the effect of DAXX on Tau oligomers in cells, a bimolecular fluorescence complementation (BiFC) assay based on the fluorescent protein Venus was performed (Shyu Y. J., et al., Biotechniques, 40:61-66, 2006). Tau was fused to the N-terminal (VN) and C-terminal (VC) fragments of Venus, respectively, generating Tau-VN and Tau-VC (Tak, H., et al., PLOS One, 8: e81682, 2013) (FIG. 15B). When Tau-VN and Tau-VC were expressed together, but not individually, in HEK293T cells, fluorescence signal was produced. Thus, Tau oligomerization brought the VN and VC moieties into close proximity to reconstitute Venus (FIG. 15B to 15D). DAXX reduced the fluorescence signal in a dose-dependent manner. At a high dose, DAXX nearly completely abrogated Tau oligomerization (FIGS. 15, C and D). DAXX showed no or minimal effect on the levels of Tau-VN and Tau-VC (FIG. 15D). Thus, DAXX blocks the formation of Tau oligomers, but does not target Tau for degradation. Collectively, these results indicate that DAXX enhances Tau solubility in cells, preventing it from spontaneous aggregation into insoluble aggregates and soluble oligomers.

DAXX Reduces Aggregation of Pathogenic polyQ Proteins

To evaluate the inhibitory effect of DAXX on huntingtin, PC12 (rat phaeochromocytoma) cells that express an enhanced green fluorescent protein (GFP)-tagged exon 1 fragment of the HD gene with 74 glutamine repeats were used (GFP-HD Q74), which is driven by a doxycycline (Dox)-dependent Tet-On promoter (referred to as PC12 HD-Q74 cells) (Wyttenbach, A., et al., Human Molecular Genetics, 10:1829-1845, 2001). Different amounts of DAXX were transfected into PC12 HD-Q74 cells and induced the expression of GFP-HD Q74 by Dox. As shown in FIG. 16A, DAXX reduced the levels of pelletable GFP-HD Q74 aggregates (PE) in a dose-dependent manner. In contrast, DAXX did not affect the levels of soluble GFP-HD Q74 in the supernatant (SN). Thus, DAXX reduces levels of the aggregated, but not soluble, GFP-HD Q74. These results, along with the potent effect of DAXX in suppressing Atxn1 82Q aggregation (Huang, L., et al., Nature, 597:132-137, 2021), indicate that DAXX is highly effective in preventing and reversing the aggregation of pathogenic polyQ proteins.

### DAXX Prevents Aggregation of ALS-Associated Proteins FUS and TDP-43

A cell-free system that inducibly generates FUS aggregates was used to investigate the effect of DAXX on FUS aggregation. A maltose-binding protein (MBP) and GFP fusion of FUS (MBP-FUS-GFP) is highly soluble. However, once the MBP moiety is cleaved of the fusion protein by the protease PreScission, the remaining portion, FUS-GFP, has a low solubility and progressively became aggregated (Hofweber, M., et al., Cell, 173:706-719, 2018). As expected, when MBP-FUS-GFP was treated with PreScission, MBP-FUS-GFP was rapidly converted to FUS-GFP (<10 min) (FIG. 16B, top panels). In the absence of DAXX, levels of SDS-soluble FUS-GFP were reduced over time (4 to 48 hours), indicating that a portion of FUS-GFP formed SDS-insoluble aggregates (FIG. 16B, top left). In contrast, in the presence of DAXX, levels of SDS-soluble FUS-GFP remained the same during the entire duration of the experiment (up to 48 hours), indicating that DAXX prevents the formation of SDS-insoluble FUS-GFP (FIG. 16B, top right). These results show that DAXX is able of blocking misfolding of proteins associated with familial ALS.

To investigate the effect of DAXX on wild-type TDP-43, DAXX and TDP-43 were co-expressed in U2OS cells. When expressed alone in U2OS cells, TDP-43 aggregated into relatively large inclusions (FIGS. 16C-16E). However, upon co-expression, DAXX markedly reduced the number and size of TDP-43 inclusions (FIGS. 16C-16E). To evaluate whether the inhibitory effect of DAXX is dependent on the polyD/E region (amino acids 449 to 499) (FIG. 16F), DAXX (365-740)—which contained the polyD/E region, and DAXX ΔD/E—was used, which lacked the polyD/E region (FIG. 16G). DAXX (365-740), but not DAXX ΔD/E, reduced the number and size of TDP-43 inclusions (FIGS. 16C-16E). Therefore, the inhibitory activity of DAXX towards TDP-43 relies on the polyD/E region.

To investigate the effect of DAXX on TDP-43 mutants associated with familial ALS, GFP-TDP43 Q331K and GFP-TDP43 M337V were used, both of which are defective in RNA binding. These mutants formed pelletable aggregates (PE) that can be detected by Western blot (FIGS. 16H and 16I). Forced expression of DAXX resulted in a reduction in aggregates formed by these TDP-43 mutants, but not the soluble TDP-43 (SN). Therefore, DAXX also reduces the levels of TDP-43 mutants. Collectively, these results indicate that DAXX is highly effective in preventing the misfolding and aggregation of wild-type and mutant TDP-43.

### Discussion

This study reveals that DAXX and other polyD/E proteins can participate in multiple aspects of PQC: preventing protein aggregation, dissolving preformed protein aggregates, and unfolding monomeric misfolded proteins. The polyD/E proteins tested here appear to have different potencies, with DAXX being stronger than SET and ANP32A, which might reflect a hierarchy within this family or a difference in substrate specificity. DAXX is particularly effective for p53 and MDM2, suggesting that polyD/E proteins may be critical for modulating conformation of their in vivo clients. Thus, DAXX and perhaps other polyD/E might have a role in both global and specific protein folding processes.

Protein folding and misfolding have been rationalized mainly in the context of hydrophobic interactions (Balchin, D., et al., Science 353, aac4354, 2016). The involvement of the polyD/E region suggests that electrostatic interactions may also contribute significantly to protein folding and misfolding, as well as the mechanism of action of proteins containing this region. Nevertheless, DAXX does not merely act as a polyanion. Rather, the other portions of DAXX likely regulate the action of the polyD/E region in a dynamic manner. The importance of electrostatic interactions, along with ATP-independence and multifunctionality, indicate that polyD/E proteins may represent a new class of protein-folding enablers, which are mechanistically distinct from canonical ATP-dependent systems as well as ATP-independent systems such as that consisting of tripartite motif (TRIM) proteins (Guo, L. et al., Mol Cell 55, 15-30, 2014; Zhu, G. et al., Cell Rep 33, 108418, 2020)

Given the prevalence of p53 mutations in human tumors, restoring thermostability and normal function of p53 mutants would be highly beneficial for cancer therapy (Bykov, V. J. N., et al., Nat Rev Cancer 18, 89-102, 2018) Nevertheless, development of small compounds to achieve such an outcome is challenging even for a single p53 mutant. This study suggests that DAXX can restore activity to a wide range of p53 mutants. Therefore, bolstering DAXX function might represent an alternative approach to therapeutically re-establish the tumor suppressive function of mutant p53.

Neurogenerative diseases are becoming increasingly prevalent as the human population ages. These diseases are progressive and eventually fatal, yet remain incurable. The potency and multifunctionality of individual polyD/E proteins such as DAXX may make them valuable for treating these diseases. While small compounds that bolster the polyD/E proteins can be beneficial, direct expression of individual polyD/E proteins may offer an alternative. Neurodegenerative diseases have been recalcitrant to conventional pharmacological treatments, in part owing to the tremendous obstacles of the blood-brain barrier and strong side effects of systematically and chronically administering small molecule drugs. Gene transfer mediated by AAVs has become a conceptually important approach for treating CNS disorders (Deverman, B. E., et al., Nature Reviews Drug Discovery, 17:641-659, 2018). AAVs can transduce the non-dividing neurons and permit permanent expression of the therapeutic gene after a single administration (Deverman, B. E., et al., Nature Reviews Drug Discovery, 17:641:659, 2018; Naldini, L., Journal of Biological Chemistry, 295:9676-9690, 2015), and the potential of AAV-mediated gene therapy is emphasized by recent positive clinical outcomes (Mendell, J. r., et al., New England Journal of Medicine, 377:1713-1722, 2017). Given their potent effect in suppressing aggregation of various proteins associated with neurodegeneration, DAXX and other polyD/E proteins might enable disease-modifying therapies.

Certain protein-misfolding diseases involve aggregated proteins in the extracellular environment. For examples, Alzheimer's disease is associated with extracellular amyloid beta (A-beta) plaques, in addition to intracellular tau aggregates, while AL amyloidosis occurs when antibody light chain proteins abnormally accumulate in the extracellular environment of organs and tissues. The present data demonstrates that DAXX can prevent and reverse amyloid beta aggregation. Therefore, one way to use DAXX (and other polyD/E proteins) is to administer these proteins to cerebrospinal fluid to clear extracellular amyloid beta plaques and other aggregates in the central nervous system. Alternatively, DAXX and other polyD/E proteins can be administered intravenously to clear aggregated in the brain as well as those in other tissues and organs.

Another way to achieve an extracellular effect is to express a version of DAXX (and other polyD/E proteins) in the cell for secretion to the extracellular environment. For example, DAXX can be fused with a secretory signal peptide, which targets proteins for translocation across the endoplasmic reticulum membrane and into the secretory pathway. Most proteins with a secretory signal peptide are eventually secreted to the extracellular environment.

### Methods

### Data Reporting

No statistical methods were used to predetermine sample size. The experiments were not randomized and the investigators were not blinded to allocation during experiments and outcome assessment.

### Antibodies and Recombinant Proteins

Antibodies against the following proteins/epitopes were purchased from the indicated sources: GAPDH (sc-47724), His (sc-8036), GST (sc-138), p53 (DO1, sc-126), MDM2 (sc-965), GFP (sc9996), DAXX (sc-8043), α-synuclein (syn211, sc-12767) (Santa Cruz Biotechnology); Flag (#14793) and DAXX (#4533S) (Cell Signaling Technology); p53 (PAb1620, #OP33; PAb240, #OP29), MDM2 (#OP46) (Calbiochem); HA (ab137838) and Luciferase (ab21176) (Abcam); GFP (GTX113617) (GeneTex); Tau (AHB0042) (Thermo Fisher Scientific); Tau (ABN454) (Millipore) and β-Amyloid 1-42 (#805509) (BioLegend). HRP-conjugated anti-rabbit IgG (#7074S) and anti-mouse IgG (#7076S) antibodies were purchased from Cell Signaling Technology; IRDye® 800CW (926-32211, anti-Rabbit) and IRDye® 680RD (926-68070, anti-Mouse) secondary antibodies from Li-Cor; anti-Flag M2 Affinity Gel (A2220), 3×Flag peptide (F4799), firefly luciferase (L9420), and Aβ42 (A9810) from Sigma Aldrich; Hsp70 (HSP72, human, ADI-NSP-555), Hsp40 (Hdj 1, human, ADI-SPP-400), and ATP regeneration solution (BML-EW9810-0100) from Enzo Life Sciences; and 6×His-ubiquitin (U-530), UBE1 (E-304), and UBE2D2 (E2-622) from Boston Biochem. α-Synuclein (#RP-003, RP-001) was purchased from Proteos. For western blot, anti-DAXX, p53, MDM2, and α-synuclein antibodies were used at 1:1000 dilution, IRDye® 800CW and IRDye® 680RD at 1:10,000 dilution, all the other antibodies at 1:2,000 dilution.

### Plasmids

Plasmids encoding HA-DAXX, Flag-DAXX, Flag-DAXX ΔD/E, Flag-p53, Flag-p53 R175H, Flag-MDM2, Flag-Atxn1 82Q, HA-Atxn1 82Q, Flag-nFluc-GFP, and Flag-nFlucDM-GFP were constructed in pRK5, and GFP-Hsp70 was constructed in pEGFP-C3, as previously described (Tang, J. et al., Nat Cell Biol 8, 855-86, 2006; Guo, L. et al., Mol Cell 55, 15-30, 2014; Zhu, G. et al., Cell Rep 33, 108418, 2020; Chu, Y. & Yang, X., Oncogene 30, 1108-1116, 2011; Chen, L. et al., Cell Rep 18, 3143-3154, 2017). Plasmid expressing LucDHis6, a Photinus pyralis luciferase variant in which the C-terminal 62 residues were replaced by SKLSYEQDGLHAGSPAALE (SEQ ID NO: 46) followed by a 6×His tag (pT7lucC-His), was a gift from Dr. Pierre Goloubinoff (Sharma, S. K., et al., Nat Chem Biol 6, 914-920, 2010). VIS and SV2 plasmids were generated by cloning α-synuclein into pBiFC-VN173 (Addgene, #22010) and pBiFC-VC155 (Addgene, #22011), respectively. DAXX shRNA plasmids were generated into pLKO.1 with oligo sequences as following: shDAXX #1, GCCTGATACCTTCCCTGACTA (SEQ ID NO: 47); shDAXX #2, GCCACACAATGCGATCCAGAA (SEQ ID NO: 48). Bacterial expression plasmid encoding GST-DAXX-6×His and GST-DAXX D/E were generated in pGEX-1ZT, a derivative of pGEX-1AT that contains additional cloning sites. Plasmids encoding ANP32A, SET, and SET deletion mutants were generated in pET28a. For protein expression in insect cells, DAXX-6×His was cloned into pFastBac-GST. Tau-VN173 was cloned in pBiFC-VN173 (Addgene plasmid #22010), and Tau-VC155 was cloned in pBiFC-VC155 (Addgene plasmid #22011). GFP-Tau P301L was made in pEGFP-N1, in which EGFP is fused to the C-terminus of Tau proteins. Flag-DAXX was constructed in pRK5 as previously described (Huang L., et al., Nature, 597:132-137, 2021; Tang, J., et al., Nature Cell Biology, 8:855-862, 2006).

### Cell Culture

HEK293T, H1299, U2OS, MDA-MB-231, SH-SY5Y and Sf9 cells were purchased from ATCC. HEK293T cells were cultured in DMEM medium, H1299 cells in RPMI-1640 medium, MDA-MB-231 cells in L15 medium, U2OS cells in McCoy's 5 medium and SH-SY5Y cells in DMEM/F12 (1:1) medium, each containing penicillin/streptomycin and 10% FBS. PC12 HD-Q74 cells were obtained (Wyttenbach, A., et al., Human Molecular Genetics, 10:1829-1845, 2001) and cultured in high glucose DMEM with 75 μg/ml hygromycin, penicillin/streptomycin, 2 mM L-glutamine, 10% heat-inactivated horse serum (HS), 5% Tet-approved fetal bovine serum (FBS), and 100 μg/ml G418. These cells were cultured at 37° C. in a humidified incubator with 5% CO2. Sf9 cells were cultured in Sf-900 III medium containing antibiotic-antimycotic at 27° C. Plasmids were transfected into cultured cells using Lipofectamine 2000 (Invitrogen).

### Protein Purification

Hsp104^{A503S} was purified as described (Jackrel, M. E. et al., Cell 156, 170-182, 2014). For expressing DAXX in bacteria and insect cells, pGEX-GST-DAXX-6×His was transformed into Rosetta 2 (Novagen), and pFB-GST-DAXX-6×His was transformed in sf9 cells. Cells were lysed with Ni-NTA lysis buffer (50 mM NaH₂PO₄, 300 mM NaCl, 10 mM imidazole at pH 8.0, 1 mM PMSF, 2 mM DTT, and 1 mg/mL lysozyme) followed by sonication. Lysates were incubated with glutathione beads (GE Healthcare, #17527901) at 4° C. for 4 h to overnight. Glutathione beads were washed sequentially with Ni-NTA lysis buffer containing 0, 0.25, 0.5, 1, 0.5, 0.25, and 0 M KCl, respectively, and twice with AcTEV buffer (50 mM Tris-HCl at pH 8.0 and 0.5 mM EDTA). The beads were then incubated at 25° C. for 2-3 h with AcTEV protease (Invitrogen, #12575015) in AcTEV buffer supplemented with 25 mM DTT. The supernatant was collected and incubated with Ni-NTA beads (Invitrogen, R90115) at 4° C. for 2-4 h. The Ni-NTA beads were washed with Ni-NTA wash buffer (50 mM NaH₂PO₄, 10 mM NaCl, and 10 mM imidazole at pH 7.0) and eluted with Ni-NTA elution buffer (50 mM NaH₂PO₄, 10 mM NaCl, and 500 mM imidazole at pH 7.0) at 4° C. for 1 h. After elution, DAXX-6×His was loaded onto PD 10 desalting columns (GE Health, GE17-0851) with Tris Buffer (20 mM Tris-HCl, 150 mM NaCl, pH 7.4, 2 mM DTT) or Sodium Phosphate Buffer (20 mM sodium phosphate buffer pH 7.4, 0.2 mM EDTA, 0.02% sodium azide). 6×His-ANP32A, 6×His-SET, and 6×His-tagged SET fragments were purified from bacteria by Ni-NTA beads. GST and GST-DAXX D/E were purified from bacteria using glutathione beads and eluted with 35 mM reduced glutathione at 4° C. for 1 h.

For purifying proteins from HEK293T cells, Flag-DAXX, Flag-p53, Flag-p53^{R175H}, Flag-MDM2, and Flag-Atxn1 82Q were transfected into HEK293T cells. Cells were lysed in IP-lysis buffer (20 mM Tris-HCl at pH 7.4, 150 mM NaCl, 0.5% Triton X-100, 0.5% NP-40, and 10% glycerol) with sonication. Supernatants were incubated with anti-Flag M2 Affinity Gel (Sigma) at 4° C. for 4 h to overnight. The Gel was washed sequentially with lysis buffers containing 0, 0.25, 0.5, 1, 0.5, 0.25, and 0 M KCl, and then with Tris buffer or sodium phosphate buffer. Recombinant proteins were eluted with 3×Flag peptide at 4° C. for 1 h.

Proteins was further purified by Mono Q (GE), Superdex 200 Increase 10/300 GL (GE), and/or Superose 6 10/300 GL columns that were driven by an NGC Chromatography System (Bio-Rad) or an AKTA FPLC system (GE Healthcare). DAXX-6×His purified from bacteria was used in FIGS. 1a, 1b, 2a, 3c; Flag-DAXX purified from HEK293T cells was used in FIGS. 4A-4G; and DAXX-6×His purified from sf9 cells was used in the other experiments unless otherwise indicated.

Flag-DAXX was purified from HEK293T cells as previously described Huang L., et al., Nature, 597:132-137, 2021; Tang, J., et al., Nature Cell Biology, 8:855-862, 2006). HEK293T cells transfected with the Flag-DAXX plasmid were lysed in IP-lysis buffer (20 mM Tris-HCl at pH 7.4, 150 mM NaCl, 0.5% Triton X-100, 0.5% NP-40, and 10% glycerol) with sonication. Supernatants were incubated with anti-Flag M2 Affinity Gel at 4° C. for 4 h to overnight. The gel was washed sequentially with lysis buffers containing additional 0, 0.25, 0.5, 1, 0.5, 0.25 and 0 M KCl, and then with Tris buffer or sodium phosphate buffer. Recombinant proteins were eluted with 3×Flag peptide at 4° C. for 1 h and then concentrated and desalted with centrifugal filters. Tau-441 (the longest isoform of human Tau) was purified as previously described (Li, W. and Lee, V. M., Biochemistry, 45:15692-15701, 2006). MBP-FUS-GFP, which was also fused to a 6×His tag, was purified by Ni-NTA resins as previously described (Hofweber, D. S., et al., Cell, 173:701-719, 2018).

### Prevention of Protein Misfolding and Aggregation

For luciferase inactivation assay, 5 or 50 nM luciferase was heated at 42° C. alone or in the presence of the indicated proteins in luciferase refolding buffer (LRB: 25 mM HEPES-KOH at pH 7.4, 150 mM KAOc, 10 mM Mg (AOc)₂, and 10 mM DTT). Heat shock proteins were used as a positive control. ATP-Mg²⁺ and an ATP-regeneration system (Enzo Life Sciences) were included in samples with heat shock proteins, but not in samples with DAXX. When indicated, the concentration is referred to that of Hsp70, and the concentrations of Hsp40 and Hsp104 were a half and twice, respectively, of that of Hsp70. Luciferase activities were measured using the Luciferase Assay System (Promega, E1500) in a Microplate Reader (BioTek). Data were acquired by BioTek Gen 5 and are expressed as percentages of the native luciferase control. To assay luciferase aggregation, 200 nM luciferase was heated at 42° C. alone or in the presence of indicated proteins in HEPES buffer (50 mM HEPES-KOH, pH7.4, 50 mM KCl, 5 mM MgCl2, 2 mM DTT). Luciferase aggregation was monitored by measuring the absorption at 600 nm in a Microplate Reader (BioTek).

Aggregation of Atxn1 82Q, p53, α-Syn, and Aβ42 were assayed by sedimentation. After incubated in the presence of the indicated proteins at 37° C. with constant shaking, the reaction mixtures were centrifuged at 17,000×g and 4° C. for 15-30 min. The SN fraction of α-Syn was treated with or without 01. mM disuccinimidyl suberate (DSS, Thermo Scientific #21555) at 25° C. for 30 min. The pellet (PE) and supernatant (SN) fractions were then separated and analyzed by western blot. For α-Syn and Aβ42, the SDS-soluble (PE) and SDS-resistant (SR) aggregates in the pellet fraction, as well as the total inputs, were also examined by dot blots on nitrocellulose membrane.

For prevention, purified Tau-441 (10 μM) was induced to form tau aggregation by Heparin (10 μM) in a reaction buffer (20 mM Tris-HCl, pH 7.4, 100 mM NaCl, 1 mM EDTA and 1 mM DTT) in the absence or presence of the indicated concentrations of Flag-DAXX at 37° C. for 24 hours. Tau fibrillization was analyzed by ThT-binding as described previously (Harischandra, D. S., et al., Science Signaling, 12: aau4543, 2019). Tau aggregation was also examined by sedimentation assay. After centrifugation at 13,000 rpm at 4° C. for 30 min, the pellet fraction was analyzed by western blot to detect SDS-soluble (PE) amorphous aggregates, and by dot blot to detect relatively large SDS-resistant (SR) fibrillar aggregates (Guo, L., et al., Molecular Cell, 55:15-30, 2014; Huang, L., et al., Nature, 597:132-137, 2021; Zhu, G., et al., Cell Reports, 33:108418, 2020). For disaggregation, preformed tau fibrils (1 μM) were incubated with or without the indicated concentrations of Flag-DAXX in a reaction solution (50 mM HEPES, pH 7.5, 50 mM KCl, 5 mM MgCl2, and 1 mM DTT) at 37° C. for 24 hour. The reaction mixtures was analyzed by ThT-binding and sedimentation assays as described above.

### Protein Fibrillization

Spontaneous and/or PFFs-induced fibrillization of α-Syn, Aβ42 and p53 was analyzed by real time quaking induced conversion assay (RT-QuIC) as previously described with modifications (Ano Bom, A. P. et al., J Biol Chem 287, 28152-28162, 2012; Yen, C. F., et al., Sci Adv 2, e1600014, 2016; Mansson, C. et al., J Biol Chem 289, 31066-31076, 2014). Preformed fibrils (PFFs) were created by incubating α-Syn (1 mg/ml), Aβ42 (10 μM), and p53^{R175H} (10 μM) at 37° C. with continuous shaking (1,000 rpm) for 7 days, 1 day, and 2 h, respectively. PFFs were sonicated for 2 min prior to use. a-Syn PFFs (133 nM) were added to human α-Syn monomers (13.3 μM) in Tris-HCl buffer (20 mM Tris-HCl, pH 7.4, 150 mM NaCl) in the presence of 10 μM ThT. Fibrillization of Aβ42 (10 μM) was performed in the sodium phosphate buffer (20 mM sodium phosphate buffer, pH 8.0, 0.2 mM EDTA, 0.02% sodium azide) with 10 μM ThT. When indicated, Aβ42 PFFs (6 nM) was added to induce fibrillization. Fibrillization of p53 and p53^{R175H} (5 μM) was performed in Tris-HCl buffer with 25 μM ThT. When indicated, p53^{R175H} PFFs (1 μM) was used to induce fibrillization. RT-QuIC assay was performed in Nunc™ MicroWell™ 96-well optical-bottom plates in a microplate reader (BioTek). The reaction mixtures were incubated at 37° C. and shaken intermittently (1-min shake-1-min rest cycle) for the indicated durations. ThT fluorescence was recorded every 2, 5 or 15 min throughout the experiment.

Fibrillization of α-Syn was also assayed by transmission electron microscopy (EM) at the Electron Microscopy Resource Laboratory at the University of Pennsylvania. Samples were stained via negative staining and scanned by FEI Tecnai-12 electron microscope.

### Disaggregation and Reactivation of Protein Aggregates

Firefly luciferase (Sigma) was heat inactivated at 42° C. for 10 min and distributed to reactions at a final concentration of 5 or 50 nM in luciferase refolding buffer (LRB: 25 mM HEPES-KOH at pH 7.4, 150 mM potassium acetate, 10 mM magnesium acetate, and 10 mM DTT). Denatured luciferase incubated with indicate proteins at 25° C. for 90 min or the indicated times. Reaction mixtures were assayed for luciferase activity, as well as for luciferase solubility by sedimentation.

α-Syn fibrils (0.5 μM of monomer concentration) were incubated GST (0.5 μM), DAXX-6×His (0.25, 0.5 or 1 μM) or HSPs (0.5 μM) in the presence of an ATP regeneration system (Enzo) for 90 min at 30° C. The samples were centrifuged at 17,000×g and 4° C. for 20 min. The supernatants were removed, and the pellets were boiled in Pellet Buffer (PB; 50 mM Tris-HCl, pH 8.0, 8 M urea, 150 mM NaCl, plus protease inhibitor cocktail). The total, supernatant, and pellet samples were then blotted on nitrocellulose membrane and incubated with anti-o-Syn antibody. Samples were quantified using ImageJ and normalized as (signal in supernatant)/(signal in pellet+signal in supernatant).

Aggregated Antx1 82Q, p53, and p53^{R175H} were generated by incubating these proteins at 37° C. shaking for 24-48 h. Aggregated MDM2 was generated by heat-inactivation at 50° C. for 10-15 min. Aggregated Atxn1 82Q, p53, and MDM2 proteins were centrifuged at 17,000 g for 15 min. Pellets were resuspended and were incubated with the indicated proteins at 25° C. Reaction mixtures were centrifuged at 17,000×g and 4° C. for 15 min. The pellet and supernatant fractions were then resuspended in sample buffer and analyzed by western blot.

### Unfoldase Assays for LucD

LucDHis6 (LucD) was inactivated by freeze-thaw circles, and monomers were isolated as previously described (Sharma, S. K., et al., Nat Chem Biol 6, 914-920, 2010). For ThT assay, misfolded LucD monomers (3 μM) were incubated with in the presence of indicated concentration of DAXX and 60 μM ThT. The addition of ThT had no detectable effects on the activity of DAXX. ThT binding was measured by microplate reader (BioTek) with excitation/emission spectrum 450/485 nm as described (Sharma, S. K., et al., Nat Chem Biol 6, 914-920, 2010). For trypsin digestion assay, LucD (50 nM) was incubated alone or in the presence of indicated proteins at 25° C. for different times. The samples were then treated with trypsin (2.5 μM) at 22° C. for 3 min and analyzed by western blot (Sharma, S. K., et al., Nat Chem Biol 6, 914-920, 2010). Steady-state kinetics analysis was performed by incubating 100 nM DAXX or HSPs with misfolded LucD monomers at increasing concentrations for 30 min. Luciferase activity was assayed. Kinetic curves were fit and kinetic parameters, Vₘₐₓ and Kₘ, were calculated by non-linear regression using the Michaelis-Menten calculated by Graphpad Prism 8.

### Circular Dichroism (CD) Spectrometer

Heat-denatured luciferase (1 μM) was incubated with and without GST and DAXX as indicated in sodium phosphate buffer for 3 h. Ellipticity was recorded between 260 and 200 nm in a quartz cuvette with 10 mm path length at 25° C. using an Aviv Circular Dichroism spectrometer. Native and heat-denatured luciferase (1 μM each) were loaded as positive and negative controls, respectively. Raw data was analyzed by CAPITO (https://data.nmr.uni-jena.de/capito/index.php). The background signal from the buffer or the buffer including DAXX was subtracted.

### ATP-Binding Assay

Binding to ATP was assayed using the ATP Affinity Test Kit (AK-102, Jena Bioscience GmbH), which contained agarose beads conjugated with ATP via the phosphate moiety (AP-ATP-agarose), the ribose moiety (EDA-ATP), or the adenine base at different positions (8AH-ATP) and (6AH-ATP agarose), and blank agarose beads without ATP conjugation. Beads (50 μL slurry) were equilibrated three times with wash buffer and then incubated with DAXX-6×His or Hsp70 (1 μg each) at 4° C. for 3 h with slight agitation. The beads were then washed 3× with wash buffer, and the bound proteins were eluted with elution buffer. Input and bound proteins were analyzed by western blot.

### Peptide Array

Cellulose-bound peptide array was made for 600 peptides representing 6 protein sequences (luciferase, p53, MDM2, H3.3, H4, and DAXX) by Biopolymers and Proteomics Core, Koch Institute, MIT. The sequence was synthesized as linear 13 amino acids in length with 10 amino acids overlapping. Recombined DAXX was used to probe the peptide assay, and peptides that could bind to DAXX were detected by anti-DAXX antibody. The array was scanned, and relative amino acid occurrence was determined. The occurrence of each amino acid in probed peptides relative to its occurrence in all peptides was determined.

### Thermal Shift Assay

Thermal shift assays was performed as described (Zhang, R. & Monsma, F., Curr Opin Drug Discov Devel 13, 389-402, 2010). Denatured p53 or MDM2 (1 μM each) was incubated with or without DAXX at 25° C. overnight, in a total volume to 9 μl. One microliter of Sypro Orange (Invitrogen, diluted 1:300 before use) was added to each sample in a 384-well plate format. The fluorescence intensity was monitored at the rate of 1° C. per min using an Applied Biosystems 7500 RT-PCR machine. DAXX signal was subtracted from the incubated samples as the background.

### In Vitro Ubiquitination

Pre-denatured Flag-p53 (20 nM) or pre-denatured Flag-MDM2 (45 nM) was incubated with Flag-DAXX (100 nM) at 25° C. for 3 h. For pre-denatured MDM2, 20 nM Zn²⁺ was added into the reaction mixtures to facilitate the folding of the Zn²⁺-chelating RING domain. The in vitro reaction was performed using 100 nM E1, 1 μM E2, and 2 μg His-ubiquitin (His-Ub) in a final volume of 20 μl reaction buffer (40 mM Tris-HCl at pH 7.6, 2.5 mM ATP, 2 mM DTT) with indicated proteins. The reaction was carried out at 37° C. for 1.5 h and was stopped by adding SDS (final concentration 1%) and boiling for 5 min. p53 and its ubiquitination was detected by western blot.

### Aβ42 Neurotoxic Assay

Cytotoxicity of Aβ42 oligomers was assessed in SH-SY5Y cells seeded in 96-well plates using the CCK8 assay (Zhu, G. et al., Cell Rep 33, 108418, 2020). Aβ42 monomers (10 μM) were incubated with DAXX-6×His (from Sf9 cells, 0.05, 0.1, 0.2, 0.4, and 0.6 μM) with constant shaking (1,000 rpm) at 37° C. for 24 h to form oligomers. The preformed oligomers were suspended in the cell culture medium for 1 h and added to SH-SY5Y cells for 24 h. Viable cells were counted by CCK8 (Dojindo, #CK04).

### Immunofluorescence and Bimolecular Fluorescence Complementation (BiFC) Assay

Cells were plated on coverslips and transfected with the indicated siRNAs, shRNAs, and/or cDNAs. Cells were fixed with 4% paraformaldehyde in PBS for 15 min at room temperature and permeabilized with PBS containing 0.25% Triton X-100 for 10 min. Cells were washed with PBS for 3 times and blocked with 1% bovine serum albumin (BSA) in PBST for 30 min at room temperature, and were then incubated with indicated primary antibodies at 4° C. overnight, followed by fluorescence-labeled secondary antibodies incubation at room temperature for 1 h. The coverslips were mounted to glass slides in medium containing 4′,6-diamidino-2-phenylindole (DAPI) (H-1200; Vector Laboratories). For BiFC assay, VIS and SV2 α-Syn plasmids were co-transfected into HEK293T cells in a molar ratio 1:1. Immunofluorescence were performed 24 h after transfection. Fluorescence images were acquired by confocal microscope (Zeiss LSM 880 with software Zeiss Zen 2.3) and analyzed by Fiji (Image J 1.52p).

### Soft Agar Colony Formation

Cells were seeded at a density of 7,500 cells/well for MDA-MB-231 cells in the top layer of 0.36% soft agar premixed with culture medium supplemented with 10% FBS in 6-well plates and incubated at 37° C. for 3 weeks. Colonies were stained with 0.05% crystal violet in 4% PFA solution for imaging and quantification as described (Zhang, Y. et al., Cell Metab 33, 94-109 e108, 2021). Images were analyzed by Fiji (Image J 1.52p).

### Gene Ontology Analysis

Gene ontology analysis was performed by Panther Classification System (http://pantherdb.org).

Analysis of polyD/E Proteins

All the reference proteomes fastas analyzed during the current study are downloaded from Uniprot database, including Homo sapiens [UP000005640], Mouse [UP00000589], C. elegans [UP000001940], Arabidopsis [UP000006548], Yeast [UP000002311], E. coli [UP000000625]. D/E enrichment region definition: Sum of the occurrence of Asp and the occurrence of Glu equal or greater than 35 in any 50 amino acid window defines as a D/E enrichment region ((D %+E %)/50>=35/50). Python notebook was used to implement a D/E enrichment region search function. Briefly, all the reference protein sequences were examined amino acid by amino acid from start to end in any possible 50 amino acids window. Once a D/E enrichment region has been found in the protein sequence, its start position, end position, counts of D, counts of E, and unique name of that protein were documented as an item. Finally, all the items of D/E enrichments were output as an excel file per species and then different isoforms of the same protein were excluded manually.

### BiFC Assay

HEK 293T cells were seeded into 6 well plates for 24 h with DMEM medium supplemented with 10% fetal bovine serum (FBS), so that cells grow to a confluence of 80-90% at the time of transfection. Cells were co-transfected with indicated plasmids. 12 h later, cells were changed with fresh medium and cultured for another 12 h. Fluorescence was observed on Revolve Microscope Demo (Echo Laboratories).

### Quantification and Statistical Analysis

Quantification of protein bands on western blots, number and size of colonies in soft-agar medium, and fluorescence signals in cells were performed by ImageJ. Statistical analysis was performed by GraphPrism 8. Individual data points were shown in plots and charts. Data were presented as mean±s.d. A unpaired Student's t-test was used to evaluate the statistical significance in the mean value between two populations (*P<0.05, **P<0.01, ***P<0.001, ns, not significant).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for treating or preventing a disease or disorder associated with misfolded protein or protein aggregates, the composition comprising a modulator of one or more poly-D/E protein.

2. The composition of claim 1, wherein the modulator increases the expression or activity of the one or more poly-D/E protein.

3. The composition of claim 1, wherein the modulator is at least one of the group consisting of a chemical compound, a protein, a peptide, a peptidomemetic, an antibody, a ribozyme, a small molecule chemical compound, a nucleic acid, a vector, and an antisense nucleic acid.

4. The composition of claim 1, wherein the modulator increases the expression or activity of at least one selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

5. The composition of claim 1, wherein the composition comprises an isolated peptide comprising one or more poly-D/E protein.

6. The composition of claim 5, wherein the isolated peptide further comprises a cell penetrating peptide (CPP) to allow for entry of the isolated peptide into a cell.

7. The composition of claim 6, wherein the CPP comprises the protein transduction domain of HIV tat.

8. The composition of claim 5, wherein the isolated peptide comprises a secretory signal peptide to direct secretion of the peptide to the extracellular environment.

9. The composition of claim 1, wherein the composition comprises an isolated nucleic acid molecule encoding one or more poly-D/E protein.

10. The composition of claim 9, wherein the isolated nucleic acid molecule comprises an expression vector.

11. The composition of claim 10, wherein the expression vector comprises an adeno-associated viral (AAV) vector.

12. The composition of claim 11, wherein the AAV vectors comprises one or more selected from the group consisting of: AAV1, AAV2, AAV3, AAV4, AAV5, AAV6, AAV7, AAV8 and AAV9.

13. The composition of claim 9, wherein the isolated nucleic acid molecule encodes a peptide comprising a secretory signal peptide and one or more poly-D/E protein.

14. The composition of claim 1, wherein the disease or disorder is one or more selected from the group consisting of:
a polyQ disorder;
a neurodegenerative disease or disorder selected from the group consisting of Spinocerebellar ataxia (SCA) Type 1 (SCA1), SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), a transmissible spongiform encephalopathy (prion disease),), a synucleinopathy, dementia with Lewy bodies (DLB), multiple system atrophy (MSA), a tauopathy, and Frontotemporal lobar degeneration (FTLD);
a disease or disorder is selected from the group consisting of AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis; and
cancer associated with p53 aggregates.

15. A method of administering a composition comprising a modulator of one or more poly-D/E protein to a subject in need thereof, comprising contacting one or more cell or tissue of the subject with the composition of claim 1.

16. A method for treating or preventing a disease or disorder associated with misfolded protein or protein aggregates in a subject in need thereof, the method comprising administering to the subject a composition comprising a modulator of the expression or activity of one or more poly-D/E protein.

17. The method of claim 16, wherein the composition comprises an isolated peptide comprising one or more poly-D/E protein selected from the group consisting of: DAXX, ANP32A, SET, HUWE1, MTEF4, MYT1, NCKX1, MYT1L, ERIP6, FAM9A, IF2P, ARMD4, PPM1G, RAGP1, NUCL, NRDC, ZFHX3, ZBT7C, ZEB1, YTDC1, ZBT47, TTBK1, KAT6B, PELP1, PTMS, TRI26, RYR1, SETLP, CLSPN, CALR, BPTF, BAZ2B, ATAD2, CFA65, CENPB, CASZ1, CCER1, DC8L2, DCAF1, AN32B, ARI4B, AN32E, UBF1, SETD1B, and VIR.

18. The method of claim 16, wherein the composition comprises an isolated nucleic acid molecule encoding one or more poly-D/E protein.

19. The method of claim 16, wherein the method comprises administering the composition to at least one cell selected from the group consisting of: a neural cell, a glial cell, and a cancer cell.

20. The method of claim 16, wherein the disease or disorder is one or more selected from the group consisting of:
a polyQ disorder;
a neurodegenerative disease or disorder selected from the group consisting of Spinocerebellar ataxia (SCA) Type 1 (SCA1), SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), a transmissible spongiform encephalopathy (prion disease),), a synucleinopathy, dementia with Lewy bodies (DLB), multiple system atrophy (MSA), a tauopathy, and Frontotemporal lobar degeneration (FTLD);
a disease or disorder is selected from the group consisting of AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis; and
cancer associated with p53 aggregates.

21. A method for producing a recombinant protein comprising administering a modulator of one or more poly-D/E protein to cell modified to express a recombinant protein.

22. The method of claim 21, wherein the modulator comprises one or more selected from the group consisting of: an isolated peptide comprising one or more poly-D/E protein and nucleic acid molecule encoding one or more poly-D/E protein.
